SEQUENCE LISTING (98 sequences)

SEQ ID NO: 1 - protein, 418 aa, Myxococcus xanthus
MKKRVGIEALAVAVPSRYVDIEDLARARGVDPAKYTAGLGAREMAVTDPGEDTVALAATAAARLIRQQDVDPSRIGMLVVGTETGIDHSKPVASHVQGLLKLPRTMRTYDTQHACYGGTAGLMAAVEWIASGAGAGKVAVVVCSDIARYGLNTAGEPTQGGGAVALLVSEQPDLLAMDVGLNGVCSMDVYDFWRPVGRREALVDGHYSITCYLEALSGAYRGWREKALAAGLVRWSDALPGEQLARIAYHVPFCKMARKAHTQLRLCDLEDAADAAASTPESREAQAKSAASYDAQVATSLGLNSRIGNVYTASLYLALAGLLQHEAGALAGQRIGLLSYGSGCAAEFYSGTVGEKAAERMAKADLEAVLARRERVSIEEYERLMKLPADAPEAVAPSPGAFRLTEIRDHRRQYAEGN

SEQ ID NO: 2 - protein, 442 aa, Myxococcus xanthus
MSDTVTSRLPGFHKLPMEERHAHLSRMFRLTPEDLQQLLGSEALQPVLANQMIENAVGTFSLPLGLGLNLQVNGRDYLVPMAVEEPSVVAAVSFAAKIVREAGGFIGEADPSLMIGQVQVSRYGDPTVATERILEHKEQILALANSFHPAMVARGGGAKDVEVRVLPAPEGPRGEPLLIVHLIIDAQEAMGANLINTMAEGVAPLIEQVTGGKVYLRILSNLADRRLARAMCRIPIPLLADFEMPAEEIAEGIAQASRFAEADPYRAATHNKGVMNGIDSVAIATGQDWRAIEAGAHAFACRNGQYRPLSTWYLEEGHLVGRIELPMALGTVGGPIKIHPGVQMALKLMQTTSVRELAMVFAAVGLAQNFAALRALGSVGIQKGHMAMHARCVAVTAGARGDWVEKIANLLVKAGHVKVEKARELLASLPAEDAAAATGTTV

SEQ ID NO: 3 - protein, 310 aa, Myxococcus xanthus
VAPRPESLSAFGAGKVILLGEHSVVYGHPALAGPLSQGVTARAVPAKACQLALPSTLSRPQRAQLTAAFARAAEVTGAPPVKVSLEADLPLAVGLGSSAALSVACARLLLQAAGKVPTPKDAARVAWAMEQEFHGTPSGVDHTTSAAEQLVLYWRKPGAAKGTGQVVESPRPLHVVVTLAGERSPTKKTVGALRERQARWPSRYERLFAEIGRVSSEGAKAVAAGDLEALGDAMNVNQGLLAALGLSSPPLEEMVYRLRELGALGAKLTGAGGDGGAVIGLFLEPKPVVTKLTRMGVRCFSSQLAGPRAS

SEQ ID NO: 4 - protein, 359 aa, Myxococcus xanthus
MERALSAPGKLFLSGEYAVLWGGVARLAAVAPRTAAYVRRRADARVHVCLEEGTLAGSTTPLGVRWEREVPAGFAFVARALDEALRAHGRASQGFDLAVAPSAVGPNGQKLGMGGSACATVLAAEGARYVLEERYDALKLALLAHTQGQGGKGSGGDVATSFAGGVLRYRRYDVAPLIEASNTGRLRAALAESPSVDVWRLPSPRVSMAYAFTGESASTRVLIGQVEARLEEAGRRSFVERSDTLGHAIEDGLSGGDFRAFSEAVKAQHALLLELGPLETEGMRRVLALAATYGAAGKLSGAGGGDGCILFAPDAQVRAEMCKGLEARGFHTLPLDAESGVRGEAQAEVRLRTWVRALS

SEQ ID NO: 5 - protein, 332 aa, Myxococcus xanthus
VSLPMKATALAHPNIALVKYWGKRDDALILPHQSSLSLTLSPLSVTTTVEFGAASDQVELNGHTAKGSERDRVLRLLELVRAQAKADLGPAKVVSRGDFPMAAGLASSAAGFAALAVAGRAAAGLPSEPRAASILARMGSGSACRSVQGGFCEWQRGERPDGEDSFAVQRFDAAHWPDVRMVVAILDRGEKEVKSRDGMKLTVDTSPYYPAWVKDAEVEVVQVREHIARRDLQALGELCERNAWRMHATSFAANPPLSYMSPGTLALILHLKEQRKKGIPVWFTLDAGPNPVLLTDAAHEVAAEALARACGALDVIRCVPGGDAELKAEHLF

SEQ ID NO: 6 - protein, 352 aa, Myxococcus xanthus
MGDDITARRKDAHLDLCSTGDVEPSGNSTLLECVKLVHCAMPEMSVEDVDLSTAFLGKRLRYPLLVTGMTGGTERAGAVNRDLALLAERHGLAFGVGSQRAMSEDASRAASFQVRQVAPTVALLGNIGMFQAIGLGVDGTRRLVDGIGADGLALHLNAGQELTQPEGDRDFQGGYRVVELLVKAFGDRLLVKETGCGIGPDVARRLVDLGVRNIDVSGLGGTSWVRVEQLRASGVQAQLGAEFSAWGIPTAAALASVRRAVGPDVHLVASGGLRTGLDAAKVLALGANLAGMALPLFRAQQAGGLEAAEAALEVILASLRQALVLTGSRSCAELRQRPRVVTGELKDWLAAL

SEQ ID NO: 7 - DNA, 1257 bp, Artificial Sequence
  hmgs Gene from Myxococcus xanthus with removed EcoRI-restriction site by insertion of a silent mutation (gaattc --> gagttc)
  CDS (1)..(1257)
atgaagaagcgcgtgggaatcgaagcgttggcggtcgcggtgccgtcccggtacgtggacatcgaagacctggcccgggcacgcggcgtggacccggccaagtacacggcgggcctgggcgccagggagatggccgtcaccgaccccggagaggacaccgtggccctcgcggccaccgcggcggcccgcctcatccgtcagcaggacgtggacccgtcccgcatcggcatgctggtggtgggcacagagacgggcatcgaccactcgaaacccgtcgcctcgcacgtgcagggcctgctgaagctgccgcgcaccatgcggacctatgacacgcagcacgcgtgttacggcggcaccgccgggctgatggcggcggtggagtggattgcgtccggcgcgggcgcgggcaaggtggccgtggtggtgtgttcggacatcgcgcgctacgggctgaacaccgcgggcgagcccacccagggcggaggcgcggtggcgctgctggtctccgagcaacccgacctgctcgccatggacgtgggcctcaacggcgtgtgcagcatggacgtgtatgacttctggcggcccgtgggccggcgggaggcgctggtggacgggcactactccatcacctgctacctggaggccctgtccggcgcttaccggggctggcgcgagaaggccctggcggcggggctggtccgctggtcggacgcgctgcccggcgaacagttggcgcgcatcgcctaccacgtgcccttctgcaagatggcccggaaggcgcacacccaactgcgcctgtgcgacctggaggatgcggcggacgcggcggcttcgacgccggagtcgcgggaggcgcaggcgaagtcggccgccagctatgacgcccaggtggcgacttcactgggactcaactcgcgcatcggaaacgtgtacaccgcgtccctctacctggcgctggcgggcctgctgcaacacgaggccggcgcgctggcgggacagcgcattggcctgctgtcctacggcagcggctgcgcggccgagttctactccggcacggtgggcgagaaggccgccgagcggatggcgaaggcggacctggaggcggtgctggcccggcgcgagcgcgtctccatcgaggaatacgagcgcctgatgaagctgccggcggatgcgcccgaggccgtggccccgtcgcccggagccttccggctgacggagattcgcgaccaccgccgccagtacgccgaagggaactga

SEQ ID NO: 8 - protein, 418 aa, Artificial Sequence; identical to SEQ ID NO: 1
  Synthetic Construct

SEQ ID NO: 9 - DNA, 1329 bp, Myxococcus xanthus
  CDS (1)..(1329)
atgtctgacaccgtgacgtcccggcttcccgggttccacaagctgccgatggaggagcgccacgcgcacctctcccgcatgttccggctcacgcccgaggacctgcagcagctgctgggctcggaggcgctccagcctgtcctggcgaaccagatgattgagaacgcggtggggaccttctccctcccgctgggcctgggcctcaacctccaggtcaacggacgtgactacctggtgcccatggcggtggaggagccgtccgtcgtggcggcggtgtccttcgcggcgaagattgtccgggaggcgggcggcttcatcggcgaggcggacccgtcgctgatgattggccaggtgcaggtgtcgcgctacggcgacccgacggtggccaccgagcgcatcctggagcacaaggagcagattctcgcgctggccaacagcttccacccggccatggtggcgcgtggcggcggggcgaaggacgtcgaggttcgcgtgctgccggccccggaagggccgcgcggcgagccgctgctcatcgtccacctcatcattgacgcccaggaggcgatgggggccaacctcatcaacaccatggcggagggcgtggcgccgctcatcgagcaggtgacgggcggcaaggtgtacctgcgcatcctctccaacctggcggaccgccggctggcgcgcgccatgtgccgcatccccatcccgctgctggcggacttcgagatgccggccgaggagatcgccgagggcatcgcccaggccagccgcttcgcggaggcggacccgtaccgcgcggccacgcacaacaagggcgtgatgaacggcattgactcggtggccatcgccacggggcaggactggcgcgccattgaagcgggcgcgcacgcgttcgcctgccgcaacgggcagtaccggccgctgtccacctggtacctggaagagggccacctggtgggccgcatcgagctgcccatggcgctggggacggtgggcgggcccatcaagatccacccgggcgtgcagatggcgctcaagctgatgcagaccacgtcggtgcgagagctcgccatggtgttcgcggcggtgggcctggcgcagaacttcgcggcgctccgggcgctgggcagcgtgggcatccagaagggccacatggcgatgcacgcgcgctgcgtggcggtgacggcgggcgcgcgaggcgactgggtggagaagatcgccaacctgctggtgaaggcgggccacgtgaaggtggagaaggcccgcgagctgctggccagcctccccgccgaggatgccgcggccgcgaccggcaccacggtctga

SEQ ID NO: 10 - protein, 442 aa, Myxococcus xanthus; identical to SEQ ID NO: 2

SEQ ID NO: 11 - DNA, 933 bp, Myxococcus xanthus
  CDS (1)..(933)
gtggctcctcgtcctgaatccttgtcggcctttggcgccggcaaggtcatcctgttgggtgagcacagcgtcgtgtacgggcacccggccctggccgggccgttgtcgcagggcgtgacggcgcgcgccgtgcccgcgaaggcgtgtcagctcgcgttgccctccacgctcagccgcccgcagcgggcgcagctcacggcggcgttcgcccgtgcggcggaggtgacgggcgcgccgccggtgaaggtgtcgctggaggcggacctgccgctggcggtgggcctgggaagctcggcggcgctgtcggtggcgtgcgcgcggttgctgctccaggcggcggggaaggtgcccacaccgaaggacgcggcgcgtgtggcgtgggcgatggagcaggagttccacggcacgccgtccggcgtggaccacaccaccagcgccgcggagcaactggtcctctactggcgcaagccgggggccgccaaggggacggggcaggtcgtggagagccccaggcccttgcacgtggtggtgacgctcgcgggtgagcgcagccccacgaagaagacggtgggcgcgctgcgggagcggcaggctcgctggccgtcgcgctacgagcgcctgttcgcggagattggacgggtgtcctcggagggcgcgaaggcggtggcggcgggagatttggaggcactgggcgacgcgatgaacgtcaaccagggcctgctcgcggcgctgggcctgtcgtcgccgccgttggaagagatggtctaccggttgcgggagctgggggcgctgggcgccaagctgaccggagctggtggagatggtggggccgtcattggcctgttcctggagcccaagcccgtggtgacgaagctcacccggatgggcgtgcgctgcttcagcagccagctcgcgggaccgcgggcgtcgtga

SEQ ID NO: 12 - protein, 310 aa, Myxococcus xanthus; identical to SEQ ID NO: 3

SEQ ID NO: 13 - DNA, 1080 bp, Myxococcus xanthus
  CDS (1)..(1080)
atggagcgcgccctctccgcgccggggaagctgttcctctccggggagtacgccgtgctgtggggcggcgtggcgcggttggccgcggtggcgccgcgcaccgccgcgtatgtccgccgccgcgcggatgcccgggtgcacgtgtgcctggaagaggggacgctggcgggaagcacgacgccgctgggcgtgcgctgggagcgtgaagtccccgcggggttcgccttcgtggcgcgggcgctggacgaggccctgcgcgcacatgggcgcgcgagccaggggttcgacctggcggtggcgccgtccgcagtggggccgaacgggcagaagctgggcatgggcggcagtgcgtgcgcgacggtgctggcggcggaaggtgcgcgctatgtgctggaagagcgctacgacgccctgaagctggcgctgctggcgcacacgcaggggcagggcgggaagggcagtggcggggacgtggcgacgagcttcgccggtggcgtgctgcgctaccggcgctacgacgtggcgcccttgattgaggcgagcaacaccgggcggttgcgcgcggcgctggcggagtctccgtcggtggacgtgtggcggctgccttcgcctcgggtgtcgatggcgtatgccttcaccggcgagagcgcctcgacgcgggtgttgattggccaggtggaggctcggctggaggaggcgggccgccggagcttcgtggagcgctcggacacactgggccatgcgattgaggacgggctgagcggcggagacttccgggccttctcggaggccgtgaaggcgcagcacgccctgctgctggagttggggccgctggagacggaaggcatgcgccgcgtgctggcgctggcggccacgtacggcgccgcaggcaagctgtccggtgcgggcggaggggacggctgcatcctgttcgcgccggatgcgcaggtccgtgcggagatgtgcaagggattggaagcccggggcttccacaccctgccgctggacgccgagtctggcgtgcgcggcgaggctcaggcggaggtccgtctccggacctgggtgcgcgcgctgagctga

SEQ ID NO: 14 - protein, 359 aa, Myxococcus xanthus; identical to SEQ ID NO: 4

SEQ ID NO: 15 - DNA, 999 bp, Myxococcus xanthus
  CDS (1)..(999)
gtgagtcttcccatgaaagccacagctctggcgcatcccaacatcgccctggtgaagtactgggggaagcgggatgacgcgttgattctgccgcaccagtccagcctgtccctcacgctgtcaccgctgtcggtgacgaccacggtggagttcggcgccgcgagcgaccaggtggagctcaacgggcacaccgcgaagggcagcgagcgcgaccgcgtgctgcggcttctggagttggtgcgcgcccaggcgaaggccgacctgggccccgcgaaggtggtgtctcgcggggacttccccatggcggcggggttggccagcagcgcggcgggcttcgcggcgctggcggtggcgggacgcgcagcggcggggttgccgtcggagccccgcgcggccagcatcctggcgcgcatgggcagcggctcggcgtgccggagcgtgcagggtgggttctgcgagtggcagcgcggcgaacgtccggatggagaggacagcttcgcggtgcagcgcttcgacgcggcccactggccggacgtgcgcatggtggtggcgattctcgaccgcggcgagaaagaggtgaagtcgcgggacgggatgaagctcacggtggacaccagcccgtactacccggcgtgggtgaaggacgccgaggtggaggtcgtgcaggtgcgcgagcacatcgccaggcgcgacctgcaggccctgggtgagctgtgtgagcgcaacgcgtggcggatgcacgcgacgtcgttcgccgcgaatccgccgctgagctacatgagccccggcacgctggcgctcatcctgcacctgaaggagcagcgcaagaagggcatcccggtgtggttcacgctggacgcggggccaaacccggtgctgctgacggacgccgcgcacgaggtggccgcggaggcgctggcccgcgcgtgcggcgcgctggatgtgattcgctgcgtgcccggcggtgacgcggagctgaaggcggagcacctcttctga

SEQ ID NO: 16 - protein, 332 aa, Myxococcus xanthus; identical to SEQ ID NO: 5

SEQ ID NO: 17 - DNA, 1059 bp, Myxococcus xanthus
  CDS (1)..(1059)
atgggcgacgacatcactgccagacgcaaggacgcgcatctcgacctttgctcgacgggggacgtcgaacccagcggaaacagcaccctgctggagtgcgtcaagctggtccactgcgcgatgccggaaatgtccgtggaggacgtggacctgtccacggccttcctgggcaagcggctgcgctacccgctgctcgtcaccggcatgacgggtgggacggagcgtgcgggtgcggtgaatcgcgacctggcgctgctcgccgagcgccacggcctggccttcggcgtgggcagccagcgcgccatgtcggaggacgcctcgcgggccgcgtccttccaggtgcggcaggtggcgcccacggtggcgctcctgggcaacatcggcatgttccaggccatcgggctgggcgtggatgggacgcgccggctggtggacggcattggcgcggacgggctggcgctgcacctcaacgcgggccaggagctgacgcagccggaaggcgaccgggacttccagggcggctaccgcgtggtggagctgctggtgaaggcctttggcgaccggctgctggtgaaggagacgggctgcggcattggcccggacgtcgcgcggcggttggtggacctgggcgtgcggaacatcgacgtgtccggtctgggcgggacgtcctgggtgcgcgtggaacaacttcgcgcgtcgggcgtacaggcacagttgggggcggagttcagcgcgtggggcattcccacggcggcggcgttggcctccgtgcgccgggccgtgggcccggacgtccacctggtggcgagcggtggcctgcgcacggggctggacgcggccaaggtgctggcgctgggggcgaacctggctggcatggcgctgccgttgttccgggcgcagcaggcgggtgggctcgaggcggcggaggcggcgctggaggtcatcctggcgagtctgcggcaggcgctcgtgctgacgggaagcagaagctgcgctgaactgagacagcggccccgggtggtcaccggagagttgaaggattggttggcggcgctgtag

SEQ ID NO: 18 - protein, 352 aa, Myxococcus xanthus; identical to SEQ ID NO: 6

SEQ ID NO: 19 - protein, 352 aa, Saccharomyces cerevisiae
MASEKEIRRERFLNVFPKLVEELNASLLAYGMPKEACDWYAHSLNYNTPGGKLNRGLSVVDTYAILSNKTVEQLGQEEYEKVAILGWCIELLQAYFLVADDMMDKSITRRGQPCWYKVPEVGEIAINDAFMLEAAIYKLLKSHFRNEKYYIDITELFHEVTFQTELGQLMDLITAPEDKVDLSKFSLKKHSFIVTFKTAYYSFYLPVALAMYVAGITDEKDLKQARDVLIPLGEYFQIQDDYLDCFGTPEQIGKIGTDIQDNKCSWVINKALELASAEQRKTLDENYGKKDSVAEAKCKKIFNDLKIEQLYHEYEESIAKDLKAKISQVDESRGFKADVLTAFLNKVYKRSK

SEQ ID NO: 20 - DNA, 1059 bp, Saccharomyces cerevisiae
  CDS (1)..(1059)
atggcttcagaaaaagaaattaggagagagagattcttgaacgttttccctaaattagtagaggaattgaacgcatcgcttttggcttacggtatgcctaaggaagcatgtgactggtatgcccactcattgaactacaacactccaggcggtaagctaaatagaggtttgtccgttgtggacacgtatgctattctctccaacaagaccgttgaacaattggggcaagaagaatacgaaaaggttgccattctaggttggtgcattgagttgttgcaggcttacttcttggtcgccgatgatatgatggacaagtccattaccagaagaggccaaccatgttggtacaaggttcctgaagttggggaaattgccatcaatgacgcattcatgttagaggctgctatctacaagcttttgaaatctcacttcagaaacgaaaaatactacatagatatcaccgaattgttccatgaggtcaccttccaaaccgaattgggccaattgatggacttaatcactgcacctgaagacaaagtcgacttgagtaagttctccctaaagaagcactccttcatagttactttcaagactgcttactattctttctacttgcctgtcgcattggccatgtacgttgccggtatcacggatgaaaaggatttgaaacaagccagagatgtcttgattccattgggtgaatacttccaaattcaagatgactacttagactgcttcggcaccccagaacagatcggtaagatcggtacagatatccaagataacaaatgttcttgggtaatcaacaaggcattggaacttgcttccgcagaacaaagaaagactttagacgaaaattacggtaagaaggactcagtcgcagaagccaaatgcaaaaagattttcaatgacttgaaaattgaacagctataccacgaatatgaagagtctattgccaaggatttgaaggccaaaatttctcaggtcgatgagtctcgtggcttcaaagctgatgtcttaactgcgttcttgaacaaagtttacaagagaagcaaatag

SEQ ID NO: 21 - protein, 352 aa, Saccharomyces cerevisiae; identical to SEQ ID NO: 19

SEQ ID NO: 22 - protein, 548 aa, Zingiber zerumbet
MERQSMALVGDKEEIIRKSFEYHPTVWGDYFIRNYSCLPLEKECMIKRVEELKDRVRNLFEETHDVLQIMILVDSIQLLGLDYHFEKEITAALRLIYEADVENYGLYEVSLRFRLLRQHGYNLSPDVFNKFKDDKGRFLPTLNGDAKGLLNLYNAAYLGTHEETILDEAISFTKCQLESLLGELEQPLAIEVSLFLETPLYRRTRRLLVRKYIPIYQEKVMRNDTILELAKLDFNLLQSLHQEEVKKITIWWNDLALTKSLKFARDRVVECYYWIVAVYFEPQYSRARVITSKAISLMSIMDDIYDNYSTLEESRLLTEAIERWEPQAVDCVPEYLKDFYLKLLKTYKDFEDELEPNEKYRIPYLQEEIKVLSRAYFQEAKWGVERYVPALEEHLLVSLITAGYFAVACASYVGLGEDATKETFEWVASSPKILKSCSIHCRLMDDITSHQREQERDHFASTVESYMKEHGTSAKVACEKLQVMVEQKWKDLNEECLRPTQVARPLIEIILNLSRAMEDIYKHKDTYTNSNTRMKDNVSLIFVESFLI

SEQ ID NO: 23 - DNA, 1647 bp, Artificial Sequence
  DNA sequence of alpha-humulene synthase zssI of Zingiber zerumbet codon-optimized for Methylobacterium extorquens AM1
  CDS (1)..(1647)
atggaacgccagtcgatggccctcgtcggcgacaaggaggagatcatccgcaagtcgttcgagtaccacccgaccgtctggggcgactacttcatccgcaactactcgtgcctcccgctcgagaaggagtgcatgatcaagcgcgtcgaagagctgaaggaccgcgtccgcaacctcttcgaagagacgcacgacgtcctccagatcatgatcctcgtcgactcgatccagctcctcggcctcgactaccacttcgagaaggagatcaccgccgccctccgcctcatctacgaggccgacgtcgagaactacggcctctacgaggtgtcgctccgcttccgcctcctccgccagcacggctacaacctctcgccggacgtgttcaacaagttcaaggacgacaagggccgcttcctcccgaccctcaacggcgacgccaagggcctcctcaacctctacaacgccgcctacctcggcacccacgaagagacgatcctcgacgaggcgatctcgttcaccaagtgccagctcgagtcgctcctcggcgagctggaacagccgctcgccatcgaggtgtcgctgttcctcgagacgccgctctatcgccgcacccgccgcctgctcgtccgcaagtacatcccgatctatcaggagaaggtcatgcgcaacgacaccatcctcgagctggccaagctggacttcaacctcctccagtcgctccaccaggaggaggtcaagaagatcaccatctggtggaacgacctcgccctcaccaagtcgctcaagttcgcccgcgaccgcgtcgtcgagtgctactactggatcgtcgccgtctacttcgagccgcagtactcgcgcgcccgcgtcatcacctcgaaggccatctcgctcatgtcgatcatggacgacatctacgacaactactcgaccctcgaagagtcgcgcctcctcaccgaggccatcgagcgctgggagccgcaggccgtcgactgcgtccccgagtacctcaaggacttctacctcaagctcctcaagacctacaaggacttcgaggacgagctggaaccgaacgagaagtaccgcatcccgtacctccaggaggagatcaaggtcctctcgcgcgcctacttccaggaggccaagtggggcgtcgagcgctacgtcccggccctcgaagagcacctcctcgtgtcgctcatcaccgccggctacttcgccgtcgcctgcgcctcgtacgtgggcctcggcgaggacgccaccaaggagacgttcgagtgggtcgcctcgtcgccgaagatcctcaagtcgtgctcgatccactgccgcctgatggacgacatcacctcgcaccagcgcgagcaggagcgcgaccacttcgcctcgaccgtggagtcgtacatgaaggagcacggcacctcggccaaggtcgcctgcgagaagctccaggtcatggtcgagcagaagtggaaggacctcaacgaggagtgcctccgcccgacccaggtcgcccgcccgctcatcgagatcatcctcaacctgtcgcgcgccatggaagacatctacaagcacaaggacacctacaccaactcgaacacccgcatgaaggacaacgtgtcgctgatcttcgtcgagtcgttcctcatctga

SEQ ID NO: 24 - protein, 548 aa, Artificial Sequence; identical to SEQ ID NO: 22
  Synthetic Construct

SEQ ID NO: 25 - DNA, 40 bp, Artificial Sequence
  Primer HMGS-fw
agtctagagaggagcgcaggatgaagaagcgcgtgggaat

SEQ ID NO: 26 - DNA, 57 bp, Artificial Sequence
  Primer HMGS-rev
atctggatccgtttaaaccctgcaggaccggtgttaactcagttcccttcggcgtac

SEQ ID NO: 27 - DNA, 29 bp, Artificial Sequence
  Primer HMGS-over-fw
gctgcgcggccgagttctactccggcacg

SEQ ID NO: 28 - DNA, 29 bp, Artificial Sequence
  Primer HMGS-over-rev
cgtgccggagtagaactcggccgcgcagc

SEQ ID NO: 29 - DNA, 42 bp, Artificial Sequence
  Primer MVA1_fw
atctggatcctaggaggaataatatgggcgacgacatcactg

SEQ ID NO: 30 - DNA, 19 bp, Artificial Sequence
  Primer MVA-SacIA-rev
aacaccatggcgagctctc

SEQ ID NO: 31 - DNA, 19 bp, Artificial Sequence
  Primer MVA-SacIA-fw
gagagctcgccatggtgtt

SEQ ID NO: 32 - DNA, 20 bp, Artificial Sequence
  Primer MVA-SacIB-rev
gtgcccgttgagctccacct

SEQ ID NO: 33 - DNA, 20 bp, Artificial Sequence
  Primer MVA-SacIB_fw
aggtggagctcaacgggcac

SEQ ID NO: 34 - DNA, 34 bp, Artificial Sequence
  Primer MVA2_rev
atcgaattcaagctttcagctcagcgcgcgcacc

SEQ ID NO: 35 - DNA, 33 bp, Artificial Sequence
  Primer pQF_MCS-fw
ctagtctgcagcttaagcatgctctagaagatc

SEQ ID NO: 36 - DNA, 33 bp, Artificial Sequence
  Primer pQF_MCS-rev
tcgagatcttctagagcatgcttaagctgcaga

SEQ ID NO: 37 - DNA, 46 bp, Artificial Sequence
  Primer ZSSI-fw
tagcatgcttaagaaggatcagtcataatggaacgccagtcgatgg

SEQ ID NO: 38 - DNA, 57 bp, Artificial Sequence
  Primer ZSSI-RBS-fw
atacactagtagcttaaggataaagaaggaggtaaaacatggaacgccagtcgatgg

SEQ ID NO: 39 - DNA, 40 bp, Artificial Sequence
  Primer ZSSI-rev
agtctagatacgtaatcgattcagatgaggaacgactcga

SEQ ID NO: 40 - DNA, 45 bp, Artificial Sequence
  Primer ERG20_fw
atcgtatcgataggagcgcaggatggcttcagaaaaagaaattag

SEQ ID NO: 41 - DNA, 68 bp, Artificial Sequence
  Primer ERG20-RB S(35k)-fw
atcgtatcgatgagaagagcagactcgatcataacaggggactagatggcttcagaaaaagaaattag

SEQ ID NO: 42 - DNA, 67 bp, Artificial Sequence
  Primer ERG20-RB S(20k)-fw
atcgtatcgatacatcaaaccaaaggactttacaggtagtagaaatggcttcagaaaaagaaattag

SEQ ID NO: 43 - DNA, 32 bp, Artificial Sequence
  Primer ERG20_rev
atcgtacgtactatttgcttctcttgtaaact

SEQ ID NO: 44 - DNA, 52 bp, Artificial Sequence
  Primer ERG20_rev-2
actatctagataaagtagaggaggattaatctatttgcttctcttgtaaact

SEQ ID NO: 45 - DNA, 35 bp, Artificial Sequence
  Primer fni-RBSopt-fw
aacctaaaattaacgaggaaagagggaggttacag

SEQ ID NO: 46 - DNA, 38 bp, Artificial Sequence
  Primer fni-RBSopt-rev
gatctgtaacctccctctttcctcgttaattttaggtt

SEQ ID NO: 47 - DNA, 33 bp, Artificial Sequence
  optimized RBS of ERG20 having a TIR of 22.000
acatcaaaccaaaggactttacaggtagtagaa

SEQ ID NO: 48 - DNA, 34 bp, Artificial Sequence
  optimized RBS of ERG20 having a TIR of 36.800
gagaagagcagactcgatcataacaggggactag

SEQ ID NO: 49 - DNA, 28 bp, Artificial Sequence
  optimized RBS sequence of Gene zssI having a TIR of 221625
agcttaaggataaagaaggaggtaaaac

SEQ ID NO: 50 - protein, 335 aa, Saccharomyces cerevisiae [sequence omitted]

SEQ ID NO: 51 - protein, 303 aa, Pantoea agglomerans
MMTVCAEQHVNFIHSDAASLLNDIEQRLDQLLPVESERDLVGAAMRDGALAPGKRIRPLLLLLAARDLGCNATPAGLLDLACAVEMVHAASLILDDMPCMDDAQLRRGRPTIHCQYGEHVAILAAVALLSKAFGVVAAAEGLTATARADAVAELSHAVGMQGLVQGQFKDLSEGDKPRSADAILMTNHYKTSTLFCASMQMASIVAEASGEAREQLHRFSLNLGQAFQLLDDLTDGMADTGKDAHQDDGKSTLVNLLGPQAVETRLRDHLRCASEHLLSACQDGYATHHFVQAWFEKKLAAVS

SEQ ID NO: 52 - protein, 393 aa, Taxus canadensis
MAYTAMAAGTQSLQLRTVASYQECNSMRSCFKLTPFKSFHGVNFNVPSLGAANCEIMGHLKLGSLPYKQCSVSSKSTKTMAQLVDLAETEKAEGKDIEFDFNEYMKSKAVAVDAALDKAIPLEYPEKIHESMRYSLLAGGKRVRPALCIAACELVGGSQDLAMPTACAMEMIHTMSLIHDDLPCMDNDDFRRGKPTNHKVFGEDTAVLAGDALLSFAFEHIAVATSKTVPSDRTLRVISELGKTIGSQGLVGGQVVDITSEGDANVDLKTLEWIHIHKTAVLLECSVVSGGILGGATEDEIARIRRYARCVGLLFQVVDDILDVTKSSEELGKTAGKDLLTDKATYPKLMGLEKAKEFAAELATRAKEELSSFDQIKAAPLLGLADYIAFRQN

SEQ ID NO: 53 - protein, 569 aa, Santalum album
MDSSTATAMTAPFIDPTDHVNLKTDTDASENRRMGNYKPSIWNYDFLQSLATHHNIVEERHLKLAEKLKGQVKFMFGAPMEPLAKLELVDVVQRLGLNHLFETEIKEALFSIYKDGSNGWWFGHLHATSLRFRLLRQCGLFIPQDVFKTFQNKTGEFDMKLCDNVKGLLSLYEASYLGWKGENILDEAKAFTTKCLKSAWENISEKWLAKRVKHALALPLHWRVPRIEARWFIEAYEQEANMNPTLLKLAKLDFNMVQSIHQKEIGELARWWVTTGLDKLAFARNNLLQSYMWSCAIASDPKFKLARETIVEIGSVLTVVDDGYDVYGSIDELDLYTSSVERWSCVEIDKLPNTLKLIFMSMFNKTNEVGLRVQHERGYNSIPTFIKAWVEQCKSYQKEARWFHGGHTPPLEEYSLNGLVSIGFPLLLITGYVAIAENEAALDKVHPLPDLLHYSSLLSRLINDIGTSPDEMARGDNLKSIHCYMNETGASEEVAREHIKGVIEENWKILNQCCFDQSQFQEPFITFNLNSVRGSHFFYEFGDGFGVTDSWTKVDMKSVLIDPIPLGEE

SEQ ID NO: 54 - protein, 569 aa, Santalum spicatum
MDSSTATATTAPFIDHTDHVNLKIDNDSSESRRMGNYKPSIWNYDFLQSLAIHHNIVEEKHLKLAEKLKGQVMSMFGAPMEPLAKLELVDVVQRLGLNHQFETEIKEALFSVYKDGSNGWWFGHLHATSLRFRLLRQCGLFIPQDVFKTFQSKTDEFDMKLCDNIKGLLSLYEASFLGWKGENILDEAKAFATKYLKNAWENISQKWLAKRVKHALALPLHWRVPRIEARWFIEAYEQEENMNPTLLKLAKLDFNMVQSIHQKEIGELARWWVTTGLDKLAFARNNLLQSYMWSCAIASDPKFKLARETIVEIGSVLTVVDDAYDVYGSMDELDHYTYSVERWSCVEIDKLPNTLKLIFMSMFNKTNEVGLRVQHERGYNGIPTFIKAWVEQCKAYQKEARWYHGGHTPPLEEYSLNGLVSIGFPLLLITGYIAIAENEAALDKVHPLPDLLHYSSLLSRLINDMGTSPDEMARGDNLKSIHCYMNETGASEEVAREHIKGIIEENWKILNQCCFDQSQFQEPFITFNLNSVRGSHFFYEFGDGFGVTDSWTKVDMKSVLIDPIPLGEE

SEQ ID NO: 55 - protein, 867 aa, Abies balsamea
MALPVYSLKSHIPITTIASAKMNYTPNKGMITANGRSRRIRLSPNKIVACAGEADRTFPSQSLEKTALFPDQFSEKNGTPSNFTPPNREFPPSFWNNDIINSITASHKVQTGDRKRIQTLISEIKNVFNSMGDGETSPSAYDTAWVARIPAVDGSEQPQFPQTLEWILQNQLKDGSWGEEFYFLAYDRLLATLACIITLTIWRTGNVQLHKGIEFFRKQVVRMDDEADNHRPSGFEIVFPAMLNEAKSLGLDLPYELPFIEQMVKKREAKLKMITTNVLYTIQTTLLYSLEGLHEIVDFDKIIKLQSKDGSFLGSPASTAAVFMQTGNTKCLEFLEFVLRKFRNHVPSDYPLDLFERLWVVDTVERLGIDRHFKKEIKDALDYVYSCWDERGIGWAKDSPIADIDDTAMGLRILRLHGYNVSPDVLKTFKDENGEFFCFMGQTQRGVTDMLNVYRCSQVAFPGETIMEEAKLCTERYLRNALENADAFDKWAIKKNIRGEVEYALKYPWHRSMPRLEVRSYIGNYGPNDVWLGKSLYMMPYISNEKYLELAKLDFNSVQSLHQEEIRELVRWCKSSGFTELKFTRDRVVETYFAVASSMFEPEFSTCRAVYTKISVLLVILDDLYDGYGSPDEIKLFSEAVKRWDLSLLEQMPDHMKICFLGLYNTVNEVAEEGRKTQGHDVLGYIRNLWEIQLAAFTREAEWSQGKYVPSFDEYIENAQVSIGVATILLITILFTEEDDILSHIDYGSKFLRLASLTARLANDIKTYQEERAHGEVVSAIQCYMKDRPEITEEEALKYVYGRMVNDLAELNSEYLKSNEMPQNCKRLVFDTARVAQLFTMEGDGLTYSDTMEIKEHIKKCLFEPAT

SEQ ID NO: 56 - DNA, 18 bp, Artificial Sequence
  optimized RBS of fni having a TIR of 65.000
gttctaggaggaataata

SEQ ID NO: 57 - DNA, 27 bp, Artificial Sequence
  optimized RBS of hmgs having a TIR of 6.345
tagagtatttctagacaggagcgcagg

SEQ ID NO: 58 - DNA, 2349 bp, Artificial Sequence
  DNA sequence of the cis-abienol synthase AbCAS from Abies balsamea codon-optimized for Methylobacterium extorquens AM1
atgaaccgcgagttcccgccgtcgttctggaacaacgacatcatcaactcgatcaccgcctcgcacaaggtccagaccggcgaccgcaagcgcatccagaccctcatctcggagatcaagaacgtgttcaactcgatgggcgacggcgagacctcgccgagcgcctacgacaccgcctgggtcgcccgcatcccggccgtggatggctcggagcagccgcagttcccgcagaccctcgagtggatcctccagaaccagctcaaggacggctcgtggggcgaggagttctacttcctcgcctacgaccgcctcctcgccaccctcgcctgcatcatcaccctcaccatctggcgcaccggcaacgtccagctccacaagggcatcgagttcttccgcaagcaggtcgtccgcatggacgacgaggccgacaaccaccgcccgtcgggcttcgagatcgtgttcccggccatgctcaacgaggccaagtcgctcggcctcgacctcccgtacgagctgccgttcatcgagcagatggtcaagaagcgcgaggccaagctcaagatgatcaccaccaacgtcctctacaccatccagaccaccctgctctactcgctcgagggcctccacgagatcgtcgacttcgacaagatcatcaagctccagtcgaaggatggcagcttcctcggctcgccggcgtcgaccgccgccgtgttcatgcagaccggcaacaccaagtgcctggagttcctcgagttcgtcctccgcaagttccgcaaccacgtcccgtcggactacccgctcgacctcttcgagcgcctctgggtcgtcgacaccgtcgagcgcctcggcatcgaccgccacttcaagaaggagatcaaggacgccctcgactacgtctactcgtgctgggacgagcgcggcatcggctgggccaaggactcgccgatcgccgacatcgacgacacggccatgggcctccgcatcctccgcctccacggctacaacgtgtcgccggacgtcctcaagaccttcaaggacgagaacggcgagttcttctgcttcatgggccagacccagcgcggcgtcaccgacatgctcaacgtctaccgctgctcgcaggtcgccttcccgggcgagaccatcatggaggaggcgaagctctgcaccgagcgctacctccgcaacgccctcgagaacgccgacgccttcgacaagtgggccatcaagaagaacatccgcggcgaggtcgagtacgccctcaagtacccgtggcaccgctcgatgccgcgcctcgaggtccgctcgtacatcggcaactacggcccgaacgacgtctggctcggcaagtcgctctacatgatgccgtacatctcgaacgagaagtacctcgagctggccaagctggacttcaactcggtccagtcgctccaccaggaggagatccgcgagctggtccgctggtgcaagtcgtcgggcttcaccgagctgaagttcacccgcgaccgcgtcgtcgagacctacttcgccgtcgcctcgtcgatgttcgagcccgagttctcgacctgccgcgccgtctacaccaagatctcggtgctcctcgtcatcctcgacgacctctacgacggctacggctcgccggacgagatcaagctgttctcggaggccgtcaagcgctgggacctctcgctcctcgagcagatgccggaccacatgaagatctgcttcctcggcctctacaacaccgtcaacgaggtcgccgaggagggccgcaagacccagggccacgacgtcctcggctacatccgcaacctctgggagatccagctcgccgccttcacccgcgaggccgagtggtcgcagggcaagtacgtcccgtcgttcgacgagtacatcgagaacgcccaggtgtcgatcggcgtcgccaccatcctgctcatcaccatcctcttcaccgaggaggacgacatcctctcgcacatcgactacggctcgaagttcctccgcctcgcctcgctcaccgcccgcctcgccaacgacatcaagacctaccaggaggagcgcgcccacggcgaggtcgtgtcggccatccagtgctacatgaaggaccgccccgagatcaccgaggaggaggccctcaagtacgtctacggccgcatggtcaacgacctggccgagctgaactcggagtacctcaagtcgaacgagatgccgcagaactgcaagcgcctcgtgttcgacaccgcccgcgtggcccagctgttcaccatggagggcgacggcctcacctactcggacaccatggagatcaaggagcacatcaagaagtgcctcttcgagccggccacctga

SEQ ID NO: 59 - DNA, 25 bp, Artificial Sequence
  optimized RBS of AbCAS with a TIR of 233,000
tattaatattaagaggaggtaataa

SEQ ID NO: 60 - DNA, 1059 bp, Artificial Sequence
  DNA sequence of the GGPP synthase ERG20F96C from Saccharomyces cerevisiae
atggcttcagaaaaagaaattaggagagagagattcttgaacgttttccctaaattagtagaggaattgaacgcatcgcttttggcttacggtatgcctaaggaagcatgtgactggtatgcccactcattgaactacaacactccaggcggtaagctaaatagaggtttgtccgttgtggacacgtatgctattctctccaacaagaccgttgaacaattggggcaagaagaatacgaaaaggttgccattctaggttggtgcattgagttgttgcaggcttactgcttggtcgccgatgatatgatggacaagtccattaccagaagaggccaaccatgttggtacaaggttcctgaagttggggaaattgccatcaatgacgcattcatgttagaggctgctatctacaagcttttgaaatctcacttcagaaacgaaaaatactacatagatatcaccgaattgttccatgaggtcaccttccaaaccgaattgggccaattgatggacttaatcactgcacctgaagacaaagtcgacttgagtaagttctccctaaagaagcactccttcatagttactttcaagactgcttactattctttctacttgcctgtcgcattggccatgtacgttgccggtatcacggatgaaaaggatttgaaacaagccagagatgtcttgattccattgggtgaatacttccaaattcaagatgactacttagactgcttcggcaccccagaacagatcggtaagatcggtacagatatccaagataacaaatgttcttgggtaatcaacaaggcattggaacttgcttccgcagaacaaagaaagactttagacgaaaattacggtaagaaggactcagtcgcagaagccaaatgcaaaaagattttcaatgacttgaaaattgaacagctataccacgaatatgaagagtctattgccaaggatttgaaggccaaaatttctcaggtcgatgagtctcgtggcttcaaagctgatgtcttaactgcgttcttgaacaaagtttacaagagaagcaaatag

SEQ ID NO: 61 - DNA, 34 bp, Artificial Sequence
  optimized RBS of ERG20F96C with a TIR of 10,000 in plasmid ppjo16
cttaaactaaccgagataggaacgaattttacaa

SEQ ID NO: 62 - DNA, 2409 bp, Artificial Sequence
  DNA sequence of the LPP synthase NtLPPS gene from Abies balsamea codon-optimized for Methylobacterium extorquens AM1
atgcaggtcatcatcacctcgtcgcaccggttcttctgccaccacctccatcagctcaagtcgccgacctcgctctcggcccagaaggccgagttcaagaagcacggcccgcgcaactggctgttccagaccgagggctcgctcctctacaagccggtccgcctcaactgcgccacctcggacgcctcgtacctcggcaacgtcaacgagtacctcgagtcggaccactcgaagaactcggaggagaaggacatccaggtcagccgcaccatccagatgaagggcctcaccgaagaaatcaagcacatgctcaactcgatggaggatggccgcctcaacgtcctcgcctacgacaccgcctgggtgtcgttcatcccgaacaccaccaacaacggcaacgaccagcgcccgatgttcccgtcgtgcctccagtggatcatcgacaaccagctctcggacggctcgtggggcgaggagatcgtgttctgcatctacgaccgcctgctcaacaccctcgtctgcgtgatcgccctcaccctctggaacacgtgcctccacaagcgcaacaagggcgtcatgttcatcaaggagaacctctcgaagctcgagaccggcgaggtcgagaacatgacctcgggcttcgagctggtgttcccgaccctcctcgagaaggcccagcagctcgacatcgacatcccgtacgatgccccggtcctcaaggacatctacgcccgccgcgaggtcaagctcacccgcatcccgaaggacgtcatccacaccatcccgaccaccgtcctcttctcgctcgagggcctccgcgacgacctcgattggcagcgcctcctcaagctccagatgccggacggcagcttcctcatctcgccggcctcgaccgccttcgccttcatggagaccaacgacgagaagtgcctcgcctacctccagaacgtcgtcgagaagtcgaacggcggcgcccgccagtacccgttcgacctcgtcacccgcctctgggccatcgaccgcctccagcgcctcggcatctcgtactacttcgccgaggagttcaaggagctgctcaaccacgtcttccgctactgggacgaggagaacggcatcttctcgggccgcaactcgaacgtgtcggacgtcgacgacacgtgcatggccatccgcctgctccgcctccacggctacgatgtctcgccggacgccctcaacaacttcaaggacggcgaccagttcgtctgcttccgcggcgaggtggatggctcgccgacccacatgttcaacctctaccgctgctcgcaggtcctcttcccgggcgagaagatcctcgaggaggccaagaacttcacctacaacttcctccagcagtgcctcgccaacaaccgctgcctcgacaagtgggtgatcgccaaggacatccccggcgagatctggtacgccctcgagttcccgtggtacgcctcgctcccgcgcgtcgaggcccgctactacatcgagcagtacggcggcgccgacgacatctggatcggcaagaccctctaccgcatgccggacgtcaacaacaacgtctacctccaggccgccaagctcgactacaaccggtgccagtcgcagcaccgcttcgagtggctcatcatgcaggagtggttcgagaagtgcaacttccagcagttcggcatctcgaagaagtacctgctcgtcagctacttcctcgccgccgcctcgatcttcgaggtcgagaagtcgcgcgagcgcctcgcctgggccaagtcgcgcatcatctgcaagatgatcacctcgtactacaacgacgaggccaccacctggaccacccgcaactcgctcctcatggagttcaaggtgtcgcacgacccgacccgcaagaacggcaacgagaccaaggagatcctcgtcctcaagaacctccgccagttcctccgccagctcagcgaggagaccttcgaggacctcggcaaggacatccaccatcagctccagaacgcctgggagacctggctcgtgttcctccgcgaggagaagaacgcctgccaggaggagaccgagctgctcgtccgcaccatcaacctctcgggcggctacatgacccacgacgagatcctcttcgacgccgactacgagaacctgtcgaacctcaccaacaaggtctgcggcaagctcaacgagctgcagaacgacaaggtcaccggcggctcgaagaacaccaacatcgagctggacatgcaggccctcgtcaagctcgtgttcggcaacacctcgtcgaacatcaaccaggacatcaagcagacgttcttcgccgtcgtcaagaccttctactactcggcccacgtgtcggaggagatcatgaacttccacatctcgaaggtcctgttccagcaggtctga

SEQ ID NO: 63 - DNA, 2301 bp, Artificial Sequence
  DNA sequence of the cis-abienol synthase NtABS gene from Abies balsamea codon-optimized for Methylobacterium extorquens AM1
atggccatctgccaccgcccgtgccgcgtccgctgctcgcattcgaccgcctcgtcgatggaggaggccaaggagcgcatccgcgagaccttcggcaagatcgagctgtcgccgtcgtcgtacgacaccgcctgggtcgccatggtcccgtcgcgctactcgatgaaccagccgtgcttcccgcagtgcctcgactggatcctcgagaaccagcgcgaggacggctcgtggggcctcaacccgtcgcacccgctcctcgtcaaggactcgctctcgtcgaccctcgcctcgctcctcgccctccgcaagtggcgcatcggcgacaaccaggtccagcgcggcctcggcttcatcgagacccacggctgggccgtcgacaacaaggaccagatctcgccgctcggcttcgagatcatcttcccgtgcatgatcaactacgccgagaagctcaacctcgacctcccgctcgacccgaacctcgtcaacatgatgctctgcgagcgcgagctgaccatcgagcgcgccctcaagaacgagttcgagggcaacatggccaacgtcgagtacttcgccgagggcctgggcgagctgtgccactggaaggagatgatgctccgccagcgccacaacggctcgctgttcgattcgccggccaccaccgccgccgccctcatctaccaccagtacgacgagaagtgcttcggctacctcaactcgatcctcaagctccacgacaactgggtcccgaccatctgcccgaccaagatccactcgaacctgttcctcgtcgacgccctccagaacctcggcgtcgaccgctacttcaagaccgaggtcaagcgcgtcctcgacgagatctaccgcctctggctcgagaagaacgaggagatcttctcggacgtcgcccactgcgccatggccttccgcctgctccgcatgaacaactacgaggtgtcgtcggaggagctggagggcttcgtcgaccaggagcacttcttcaccacctcgtcgggcaagctcatgaaccacgtcgccatcctcgagctgcaccgcgcctcgcaggtcgcgatccacgagcgcaaggaccacatcctcgacaagatctcgacctggacccgcaacttcatggagcagaagctcctcgacaagcacatcccggaccgctcgaagaaggagatggagttcgccatgcgcaagttctacggcaccttcgaccgcgtcgagacccgccgctacatcgagtcgtacaagatggactcgttcaagatcctcaaggccgcctaccgctcgtcgggcatcaacaacatcgacctgctcaagttctcggagcacgacttcaacctctgccagacccgccacaaggaggagctgcagcagatgaagcgctggttcaccgactgcaagctcgagcaggtcggcctctcgcagcagtacctctacacctcgtacttcatcatcgccgccatcctcttcgagcccgagtacgccgacgcccgcctcgcctacgccaagtacgccatcatcatcaccgccgtcgacgacttcttcgactgcttcatctgcaaggaggagctgcagaacatcatcgagctggtcgagcgctgggagggctactcgaccgtcggcttccgctcggagcgcgtccgcatcttcttcctcgccctctacaagatggtcgaggagatcgccgccaaggccgagaccaagcagggccgctgcgtcaaggaccacctcatcaacctctggatcgacatgctgaagtgcatgctcgtcgagctggacctctggaagatcaagtcgaccaccccgagcatcgaggagtacctctcggtcgcctgcgtcaccatcggcgtcccgtgcttcgtcctgacctcgctctacctcctcggcccgaagctctcgaaggacgtcatcgagtcgtcggaggtgtcggccctctgcaactgcaccgccgccgtggcccgcctcatcaacgacatccactcgtacaagcgcgagcaggccgagtcgtcgaccaacatggtgtcgatcctcatcacccagtcgcagggcaccatctcggaggaggaggccatccgccagatcaaggagatgatggagtcgaagcgccgcgagctgctcggcatggtcctccagaacaaggagtcgcagctcccgcaggtctgcaaggacctcttctggaccaccatcaacgccgcctactcgatccacacccacggcgacggctaccgcttccccgaggagttcaagaaccacatcaacgacgtcatctacaagccgctcaaccagtactcgccgtga

SEQ ID NO: 64 - DNA, 31 bp, Artificial Sequence
  optimized RBS of NtLPPS with a TIR of 145,000 in plasmid ppjo16
caacggcccttacaaaaggaggttaattatt

SEQ ID NO: 65 - DNA, 33 bp, Artificial Sequence
  optimized RBS of NtABS with a TIR of 130,000 in plasmid ppjo16
gatagaaacccttaattaagaaggaggtcctta

SEQ ID NO: 66 - DNA, 35 bp, Artificial Sequence
  optimized RBS of ERG20F96C with a TIR of 9,500 in plasmid ppjo17
aaccactaagaacacagacttatacacaggaggat

SEQ ID NO: 67 - DNA, 196 bp, Artificial Sequence
  promoter region of plasmid ppjo16 including RBS AbCAS, beginning directly after CymR
tcggggtctctccctgctagcatcagggttattgtatcatgagcggatacatatgtcaatgtaccggaacaaacagacaatctggtctgtttgtacagcattgacgggcccggccaccccgcgtagaagcgcgccacaacaaacagacaatctggtctgtttgtaactagttattaatattaagaggaggtaataa

SEQ ID NO: 68 - DNA, 168 bp, Artificial Sequence
  promoter region of plasmid ppjo16 from clone 16s6 including RBS AbCAS, beginning directly after CymR
tcggggtctctccctgctagcatcagggttattgtatcatgagcggatacatactggtctgtttgtacagcattgacgggcccggccaccccgcgtagaagcgcgccacaacaaacagacaatctggtctgtttgtaactagttattaatattaagaggaggtaataa

SEQ ID NO: 69 - DNA, 1710 bp, Artificial Sequence
  DNA sequence of the santaledne synthase SspiSSY from Santalum spicatum codon-optimized for Methylobacterium extorquens AM1
atggactcgtcgaccgccaccgccaccaccgccccgttcatcgaccacaccgaccacgtcaacctcaagatcgacaacgactcgtcggagtcgcgccgcatgggcaactacaagccgtcgatctggaactacgacttcctccagtcgctcgccatccaccacaacatcgtcgaggagaagcacctcaagctcgccgagaagctcaagggccaggtcatgtcgatgttcggcgccccgatggagccgctcgccaagctcgagctggtcgacgtcgtgcagcgcctcggcctcaaccaccagttcgagaccgagatcaaggaggccctcttctcggtctacaaggacggctcgaacggctggtggttcggccacctccacgccacctcgctccgcttccgcctcctccgccagtgcggcctcttcatcccgcaggacgtgttcaagaccttccagagcaagaccgacgagttcgacatgaagctctgcgacaacatcaagggcctgctctcgctctacgaggcgtcgttcctcggctggaagggcgagaacatcctcgacgaggccaaggccttcgccaccaagtacctcaagaacgcctgggagaacatctcgcagaagtggctcgccaagcgcgtcaagcacgccctcgccctcccgctgcattggcgcgtcccgcgcatcgaggcccgctggttcatcgaggcctacgagcaggaggagaacatgaacccgaccctgctcaagctggccaagctcgacttcaacatggtccagtcgatccaccagaaggagatcggcgagctggcccggtggtgggtcaccaccggcctcgataagctcgccttcgcccgcaacaacctcctccagtcgtacatgtggtcgtgcgcgatcgcctcggacccgaagttcaagctcgcccgcgagaccatcgtcgagatcggctcggtcctcaccgtcgtggacgacgcctacgacgtctacggctcgatggacgagctggaccactacacctactcggtcgagcgctggtcgtgcgtcgagatcgacaagctcccgaacaccctcaagctcatcttcatgagcatgttcaacaagaccaacgaggtcggcctccgcgtccagcacgagcgcggctacaacggcatcccgaccttcatcaaggcctgggtcgagcagtgcaaggcctaccagaaggaggcgcgctggtaccatggcggccacaccccgccgctcgaggagtactcgctcaacggcctcgtgtcgatcggcttcccgctcctcctcatcaccggctacatcgcgatcgccgagaacgaggccgccctcgacaaggtccacccgctcccggatctgctccactactcgtcgctcctctcgcgcctcatcaacgacatgggcacctcgccggacgagatggcccgcggcgataacctcaagtcgatccactgctacatgaacgagaccggcgcctcggaggaggtcgcccgcgagcacatcaagggcatcatcgaggagaactggaagatcctcaaccagtgctgcttcgaccagtcgcagttccaggagccgttcatcaccttcaacctcaactcggtccgcggctcgcacttcttctacgagttcggcgacggcttcggcgtcaccgactcgtggaccaaggtcgacatgaagtcggtcctcatcgacccgatcccgctcggcgaggagtga

SEQ ID NO: 70 - DNA, 32 bp, Artificial Sequence
  optimized RBS of SSpiSSY with a TIR of 44,000 in plasmid ppjo01 and ppjo1_woMVA
tgttacacccacagaacaaacccgaggtaact

SEQ ID NO: 71 - DNA, 33 bp, Artificial Sequence; identical to SEQ ID NO: 47
  optimized RBS of ERG20 with a TIR of 20,000 in plasmid ppjo01 and ppjo1_woMVA

SEQ ID NO: 72 - DNA, 1656 bp, Artificial Sequence
  DNA sequence of the santalene synthase SanSyn from Clausena lansium codon-optimized for Methylobacterium extorquens AM1
atgtcgacccagcaggtcagctcggagaacatcgtccgcaacgccgccaacttccacccgaacatctggggcaaccacttcctcacgtgcccgtcgcagaccatcgactcgtggacccagcagcaccacaaggaactcaaggaagaggtccgcaagatgatggtgtcggacgccaacaagccggcccagcgcctccgcctcatcgacaccgtccagcgcctcggcgtcgcctaccacttcgagaaggaaatcgacgacgccctcgagaagatcggccacgacccgttcgacgacaaggacgacctctacatcgtgtcgctctgcttccgcctcctccgccagcacggcatcaagatctcgtgcgacgtgttcgagaagttcaaggacgacgacggcaagttcaaggcctcgctcatgaacgacgtccagggcatgctctcgctctacgaagccgcccacctcgccatccacggcgaggacatcctcgacgaggccatcgtgttcaccaccacccacctcaagtcgaccgtgtcgaactcgccggtcaactcgaccttcgccgagcagatccgccactcgctccgcgtcccgctccgcaaggccgtcccgcgcctcgaatcgcgctacttcctcgacatctactcgcgcgacgacctccacgacaagaccctgctcaacttcgccaagctcgacttcaacatcctccaggccatgcaccagaaggaagcctcggagatgacccggtggtggcgcgacttcgacttcctcaagaagctcccgtacatccgcgaccgcgtcgtcgagctgtacttctggatcctcgtcggcgtgtcgtaccagccgaagttctcgaccggccgcatcttcctctcgaagatcatctgcctcgaaaccctcgtcgacgacaccttcgacgcctacggcaccttcgacgagctggccatcttcaccgaggccgtcacccgctgggatctcggccaccgcgacgccctccccgagtacatgaagttcatcttcaagaccctcatcgacgtctactcggaggccgagcaggaactcgccaaggaaggccgctcgtactcgatccactacgccatccgctcgttccaggaactcgtgatgaagtacttctgcgaggccaagtggctcaacaagggctacgtcccgtcgctcgacgactacaagtcggtgtcgctccgctcgatcggcttcctcccgatcgccgtcgcctcgttcgtgttcatgggcgatatcgcgaccaaggaagtgttcgagtgggagatgaacaacccgaagatcatcatcgccgccgaaacgatcttccgcttcctcgacgatatcgccggccaccgcttcgagcagaagcgcgagcactcgccgtcggccatcgagtgctacaagaaccagcacggcgtgtcggaggaagaggccgtcaaggccctctcgctcgaggtcgccaactcgtggaaggatatcaacgaggaactcctcctcaacccgatggccatcccgctcccgctcctccaggtcatcctcgacctctcgcgctcggccgacttcatgtacggcaacgcccaggaccgcttcacccactcgaccatgatgaaggaccaggtcgacctcgtgctcaaggacccggtcaagctcgacgactga

SEQ ID NO: 73 - DNA, 35 bp, Artificial Sequence
  optimized RBS of SanSyn with a TIR of 233,000 in plasmid ppjo03
gaagaaggaggtagtcataaagaaggaggtaacta

SEQ ID NO: 74 - DNA, 34 bp, Artificial Sequence
  optimized RBS of ERG20 with a TIR of 22,000 in plasmid ppjo03
tccccagcgcgccccccaattcaggataacatag

SEQ ID NO: 75 - DNA, 34 bp, Artificial Sequence
  optimized RBS of ERG20 with a TIR of 53,000 in plasmid ppjo04 and ppjo1_woMVA
aaacatagcatattagcagattaaggacatacgt

SEQ ID NO: 76 - DNA, 35 bp, Artificial Sequence
  optimized RBS of SSpiSSY with a TIR of 402,000 in plasmids ppjo05 and ppjo06
ccccttcccttatttaaaccagaggaggtaacaaa

SEQ ID NO: 77 - DNA, 32 bp, Artificial Sequence
  optimized RBS of ERG20 with a TIR of 1,344,000 in plasmid ppjo06
aaccaaataggattagcacagaagggggtaat

SEQ ID NO: 78 - DNA, 26 bp, Artificial Sequence
  Primer pj05
aacagacaatctggtctgtttgtaac

SEQ ID NO: 79 - DNA, 65 bp, Artificial Sequence
  Primer pj10
tcttcatcctgcgctcctgtctagaaatactctaattaatctatttgcttctcttgtaaactttg

SEQ ID NO: 80 - DNA, 20 bp, Artificial Sequence
  Primer pj16
atcggcgaccaagcagtaag

SEQ ID NO: 81 - DNA, 20 bp, Artificial Sequence
  Primer pj17
ttactgcttggtcgccgatg

SEQ ID NO: 82 - DNA, 39 bp, Artificial Sequence
  Primer pj25
cgttcctatctcggttagtttaagatcgattcaggtggc

SEQ ID NO: 83 - DNA, 61 bp, Artificial Sequence
  Primer pj26
cttaaactaaccgagataggaacgaattttacaatatggcttcagaaaaagaaattaggag

SEQ ID NO: 84 - DNA, 51 bp, Artificial Sequence
  Primer pj27
gacctccttcttaattaagggtttctatctacgtatcagacctgctggaac

SEQ ID NO: 85 - DNA, 27 bp, Artificial Sequence
  Primer pj28
gatagaaacccttaattaagaaggagg

SEQ ID NO: 86 - DNA, 40 bp, Artificial Sequence
  Primer pj29
tataagtctgtgttcttagtggttatcgattcacggcgag

SEQ ID NO: 87 - DNA, 61 bp, Artificial Sequence
  Primer pj30
aaccactaagaacacagacttatacacaggaggatatggcttcagaaaaagaaattaggag

SEQ ID NO: 88 - DNA, 60 bp, Artificial Sequence
  Primer SspiSSY_RBSopt_fw
acgaactagttgttacacccacagaacaaacccgaggtaactatggactcgtcgaccgcc

SEQ ID NO: 89 - DNA, 29 bp, Artificial Sequence
  Primer SspiSSY_rev
atcgtatcgattcactcctcgccgagcgg

SEQ ID NO: 90 - DNA, 81 bp, Artificial Sequence
  Primer pj01
gacaatctggtctgtttgtaactagtccccttcccttatttaaaccagaggaggtaacaaaatggactcgtcgaccgccac

SEQ ID NO: 91 - DNA, 20 bp, Artificial Sequence
  Primer pj06
tgggcataccagtcacatgc

SEQ ID NO: 92 - DNA, 67 bp, Artificial Sequence
  Primer ERG20-fus_fw
acgaactagtaaacatagcatattagcagattaaggacatacgtatggcttcagaaaaagaaattag

SEQ ID NO: 93 - DNA, 56 bp, Artificial Sequence
  Primer ERG20-fus_rev
actaggatccgccgccacccgagccaccgccacctttgcttctcttgtaaactttg

SEQ ID NO: 94 - DNA, 62 bp, Artificial Sequence
  Primer pj08
tttctgaagccatggatccgccgccacccgagccaccgccaccctcctcgccgagcgggatc

SEQ ID NO: 95 - DNA, 32 bp, Artificial Sequence
  Primer pj09
ggatccatggcttcagaaaaagaaattaggag

SEQ ID NO: 96 - DNA, 46 bp, Artificial Sequence
  Primer pj77
cttctgtgctaatcctatttggttatcgattcactcctcgccgagc

SEQ ID NO: 97 - DNA, 62 bp, Artificial Sequence
  Primer pj78
gataaccaaataggattagcacagaagggggtaataatggcttcagaaaaagaaattaggag

SEQ ID NO: 98 - DNA, 38 bp, Artificial Sequence
  optimized RBS of hmgs with a TIR of 189
attaatcctcctctactttatctagagaggagcgcagg

(19) United States
(12) Patent Application Publication
(10) Pub. No.: US 2018/0105838 A1
(43) Pub. Date: Apr. 19, 2018

(54) PROCESS FOR DE NOVO MICROBIAL SYNTHESIS OF TERPENES

(71) Applicant: BASF SE, Ludwigshafen (DE)

(72) Inventors: Jens SCHRADER, Frankfurt (DE); Markus BUCHHAUPT, Bad Vilbel (DE); Frank SONNTAG, Langen (DE); Cora KRONER, Frankfurt (DE); Heike BRÜSER, Speyer (DE); Hartwig SCHRÖDER, Nußloch (DE); Ralf PELZER, Fürstenberg (DE)

(21) Appl. No.: 15/557,370

(22) PCT Filed: Mar. 11, 2016
(86) PCT No.: PCT/EP2016/055239

§ 371 Date: Sep. 11, 2017

(30) Foreign Application Priority Data
Mar. 11, 2015 (DE) 102015103608.8

(51) Int. Cl. C12P 5/00; C12N 9/88; C12N 9/10; C12N 9/04; C12N 9/12; C12N 9/90

(52) U.S. Cl. CPC C12P 5/007; C12N 9/88; C12Y 402/03104; C12N 9/1025; C12Y 203/0301; C12N 9/0006; C12Y 503/03002; C12N 9/1205; C12Y 207/01036; C12N 9/1229; C12Y 207/04002; C12Y 401/01033; C12N 9/90; C12Y 101/01034

(57) ABSTRACT

The invention relates to microbial terpene production. Known methods for microbial production of terpenes are mostly based on the direct conversion of sugars. Therefore alternative substrates, in particular alternative carbon sources, for use in microbial terpene production were desirable. The invention relates to a methylotrophic bacterium containing recombinant DNA coding for at least one polypeptide with enzymatic activity for heterologous expression in said bacterium, wherein said at least one polypeptide with enzymatic activity is selected from the group consisting an enzyme of a heterologous mevalonate pathway, a heterologous terpene synthase and optionally a heterologous synthase of a prenyl diphosphate precursor. The invention further relates in particular to a method for de novo microbial synthesis of sesquiterpenes or diterpenes from methanol and/or ethanol.

The invention relates to a methylotrophic bacterium, a method for de novo microbial synthesis of sesquiterpenes or diterpenes from methanol and/or ethanol and the use of the methylotrophic bacterium for the de novo microbial synthesis of terpenes from methanol and/or ethanol. The invention relates to the field of white biotechnology.

The microbial synthesis of terpenes as an environmentally friendly possibility for production of aroma, perfume and cosmetic substances and biofuel substances has already been described for various microorganisms such as Escherichia coli or Saccharomyces cerevisiae (Martin et al., 2003, Nature Biotechnology 21, 796-802; Asadollahi et al., 2008, Biotechnology and Bioengineering 99, 666-677; Chandran et al., 2011, Process Biochemistry 46, 1703-1710). In these a considerable growth potential is ascribed to microbial terpene production in the coming years, which results above all from scarcity of fossil resources and the growing world population coupled with the need for an environmentally friendly synthesis of chemical substances (Peralta-Yahya et al., 2010, Biotechnol J 5, 147-62; Ajikumar et al., 2008, Molecular pharmaceutics 5, 167-90).

Known methods for microbial production of terpenes are mostly based on the direct conversion of sugars, in particular glucose, or of substrates which are in competition with food production, such as glycerin, or complex substrates such as protein hydrolyzates (Yoon et al., 2009, Journal of Biotechnology, 140, 218-226; Sarria et al., 2014, ACS Synthetic Biology 3 (7), 466-475). The utilization of such substances as substrates for biotechnology is attended by various disadvantages, which as well as the ethical component relate above all to fluctuating and presumably increasing price levels in the future and regional and seasonal factors. Further, the use of complex or sugar-containing media is always attended by increased costs in the product processing and sterility requirements. Hence alternative substrates, in particular alternative carbon sources, are desirable for use in biotechnology, which compensate for as many as possible of the aforesaid disadvantages.

The microbial production of terpenes, in particular amorpha-4,11-dienes, or terpene mixtures is described in US 2008/0274523 A1. However, according to this only monosaccharide glucose is used as the carbon source. In particular, no alternative carbon source for the fermentation is described therein. Furthermore, according to US 2008/0274523 A1 the heterologous expression of an acetoacetyl-CoA synthase (acetoacetyl-CoA thiolase) is necessary.

According to US 2003/0148479 A1, a microbial biosynthesis of isopentenyl pyrophosphate (IPP) in E. coli is proposed, wherein culturing is performed in LB media. A heterologous expression of an acetoacetyl-CoA synthase (acetoacetyl-CoA thiolase) is necessary in this, and moreover various intermediates of the mevalonate pathway were added to the medium. An alternative carbon source for the fermentation is not proposed.

US 2011/0229958 A1 shows microorganisms for the production of isoprene compounds in E. coli. A heterologous expression of an acetoacetyl-CoA synthase (acetoacetyl-CoA thiolase) is once again necessary and mevalonate was added to the medium. An inexpensive alternative carbon source for the fermentation is not proposed.

With WO 2014/014339 A2, recombinant Rhodobacter host cells for monoterpene synthesis are described. As the carbon source, a sugar not characterized in more detail is proposed. An alternative carbon source for the fermentation is not mentioned.

A de novo production of the monoterpene geranic acid in Pseudomonas putida has been proposed (Mi et al., 2014, Microbial Cell Factories 13:170). However, according to this only glycerin in an LB-containing medium is used as the carbon source. In particular, no alternative carbon source for the fermentation is proposed therein.

The use of complex media or of those the composition whereof is not adequately characterized impedes simple and inexpensive workup of the desired product.

The purpose of the present invention thus according to a first aspect consisted in overcoming the disadvantages of the known recombinant microorganisms with regard to the biosynthesis of terpenes. According to a further aspect of the present invention, bacteria are to be provided which enable a de novo microbial synthesis of terpenes from an alternative carbon source. Here the bacteria should ideally be able to grow on the alternative carbon source as the sole carbon source, in particular an addition of cost-intensive substrate additives, such as aceto-acetate or D,L-mevalonate, should not be necessary. According to a further aspect, a fermentation method for de novo microbial synthesis of terpenes from an alternative carbon source which enables simple downstream purification of the terpene products obtained should be provided. In particular, sesquiterpenes and diterpenes should be producible in high yield. According to a further aspect, the conversion and yield of the method both in the shaker flask and also in the fermenter on scale-up for the biotechnological use should be very promising or adequate.

The problems are solved by the embodiments described in the claims and below.

A first embodiment of the invention relates to a methylotrophic bacterium containing recombinant DNA coding for at least one polypeptide with enzymatic activity for expression in said bacterium, wherein said at least one polypeptide with enzymatic activity is selected from the group consisting of
at least one enzyme of a heterologous mevalonate pathway selected from the group consisting of hydroxymethylglutaryl-CoA synthase (HMG-CoA synthase), hydroxymethylglutaryl-CoA reductase (HMG-CoA reductase), mevalonate kinase, phosphomevalonate kinase, pyrophosphomevalonate decarboxylase and isopentenyl pyrophosphate isomerase;
a heterologous terpene synthase and
a synthase of a prenyl diphosphate precursor.

In particular, the invention also relates to a methylotrophic bacterium containing a heterologous terpene synthase and recombinant DNA coding for at least one polypeptide with enzymatic activity for expression in said bacterium, characterized in that said at least one polypeptide with enzymatic activity is selected from the group consisting of
at least one enzyme of a heterologous mevalonate pathway selected from the group consisting of hydroxymethylglutaryl-CoA synthase (HMG-CoA synthase), hydroxymethylglutaryl-CoA reductase (HMG-CoA reductase), mevalonate kinase, phosphomevalonate kinase, pyrophosphomevalonate decarboxylase and isopentenyl pyrophosphate isomerase; and
a synthase of a prenyl diphosphate precursor.

Likewise, the invention also relates to a methylotrophic bacterium containing a heterologous hydroxymethylglutaryl-CoA synthase (HMG-CoA synthase) and a hydroxymethylglutaryl-CoA reductase (HMG-CoA reductase) as enzymes of a heterologous mevalonate pathway and recombinant DNA coding for at least one polypeptide with enzymatic activity for expression in said bacterium, characterized in that said at least one polypeptide with enzymatic activity is selected from the group consisting of
at least one further enzyme of a heterologous mevalonate pathway selected from the group consisting of mevalonate kinase, phosphomevalonate kinase, pyrophosphomevalonate decarboxylase and isopentenyl pyrophosphate isomerase;
a heterologous terpene synthase and
a synthase of a prenyl diphosphate precursor.

Especially preferably, the bacterium according to the invention contains at least the following enzymes:
heterologous hydroxymethylglutaryl-CoA synthase (HMG-CoA synthase) and hydroxymethylglutaryl-CoA reductase (HMG-CoA reductase) and at least one enzyme selected from mevalonate kinase, phosphomevalonate kinase, pyrophosphomevalonate decarboxylase and isopentenyl pyrophosphate isomerase and especially preferably all these enzymes; and
a heterologous terpene synthase,

Most preferably the bacterium additionally also contains a synthase of a prenyl diphosphate precursor.

In the sense of the invention “heterologous” should be understood to mean an enzyme or a group of enzymes, for example those of the mevalonate pathway, which do not naturally occur in an organism, which now according to the invention is to contain the enzyme or the group of enzymes. Thus the heterologous terpene synthase or the enzymes of the heterologous mevalonate pathway should not occur in the methylotrophic bacterium according to the invention, but rather derive from one or more other species.

The bacterium according to the invention surprisingly enables a de novo microbial synthesis of terpenes from an alternative carbon source, such as methanol and/or ethanol. Said bacterium can, with heterologously expressed enzymes of the mevalonate pathway (MVA pathway) otherwise not naturally occurring in this bacterium, grow on methanol and/or ethanol as the sole carbon source and synthesize desired terpenes de novo in high yield.

A particular feature of the methylotrophic bacterium used consists in the presence of the molecule acetoacetyl-CoA in the primary metabolism, here the ethylmalonyl-CoA pathway (EMCP). Acetoacetyl-CoA is the first molecule in the mevalonate pathway. The viability of the recombinant methylotrophic bacterium according to the invention was in no way to be expected. Thus on withdrawal of metabolites of the primary metabolism, considerable flux imbalances can certainly be assumed. In this respect, the growth of the bacterium according to the invention with at least one heterologously expressed enzyme of the mevalonate pathway otherwise not occurring naturally in this bacterium on methanol and/or ethanol is surprising. Furthermore, the presence of the molecule acetoacetyl-CoA in the primary metabolism makes a heterologous expression of an acetoacetyl-CoA synthase superfluous. According to a further preferred modification of the invention, the methylotrophic bacterium contains no recombinant DNA coding for heterologous expression of an acetoacetyl-CoA synthase (acetoacetyl-CoA thiolase).

The synthase of a prenyl diphosphate precursor in the sense of the present invention in particular enzymatically converts isopentenyl diphosphate (IPP) and dimethylallyl diphosphate (DMAPP) to a prenyl diphosphate precursor, wherein the prenyl diphosphate precursor is preferably selected from the group consisting of farnesyl diphosphate (FPP) (C₁₅) and geranyl-geranyl diphosphate (GGPP) (C₂₀).

The acyclic prenyl diphosphates formed (synonymous here with isoprenyl diphosphates)—FPP and GGPP—are the precursors of a large number of terpenes. The substrates of the heterologous terpene synthase are preferably selected from said prenyl diphosphate precursors.

According to a preferred embodiment, the methylotrophic bacterium according to the invention contains recombinant DNA coding for polypeptides with enzymatic activity for heterologous expression in said bacterium, wherein the polypeptides with enzymatic activity include the following enzymes:
the enzymes of a heterologous mevalonate pathway (MVA pathway), namely hydroxymethylglutaryl-CoA synthase (HMG-CoA synthase), hydroxymethylglutaryl-CoA reductase (HMG-CoA reductase), mevalonate kinase, phosphomevalonate kinase, pyrophosphomevalonate decarboxylase and isopentenyl pyrophosphate isomerase;
a heterologous terpene synthase and
an, in particular heterologous, synthase of a prenyl diphosphate precursor.

A preferred bacterium according to the invention is characterized in that the at least one enzyme of the heterologous mevalonate pathway—namely an enzyme selected from the group consisting of hydroxymethylglutaryl-CoA synthase (HMG-CoA synthase), hydroxy-methylglutaryl-CoA reductase (HMG-CoA reductase), mevalonate kinase, phosphomevalonate kinase, pyrophosphomevalonate decarboxylase and isopentenyl pyrophosphate isomerase contains a peptide sequence with an identity of respectively at least 60% to the peptide sequence according to SEQ ID No. 1, SEQ ID No. 2, SEQ ID No. 3, SEQ ID No. 4, SEQ ID No. 5 or SEQ ID No. 6 or is encoded by a nucleic acid sequence which is capable of hybridizing under stringent hybridization conditions with the corresponding nucleic acid sequence coding for the specific peptide sequences.

By enzymes which contain a peptide sequence with an identity of respectively at least 60% to the peptide sequence according to SEQ ID No. 1, SEQ ID No. 2, SEQ ID No. 3, SEQ ID No. 4, SEQ ID No. 5 or SEQ ID No. 6, it should preferably be understood that the enzymes contain a peptide sequence which is in each case at least 65%, at least 70%, at least 75%, at least 80%, at least 85%, at least 90%, at least 95%, at least 96%, at least 97%, at least 98% or at least 99% identical with one of the specific peptide sequences according to SEQ ID No. 1. SEQ ID No. 2, SEQ ID No. 3, SEQ ID No. 4, SEQ ID No. 5 or SEQ ID No. 6. It goes without saying that such variants of the enzymes with specific peptide sequence should also preferably essentially have the biological activity of the aforesaid enzymes with specific peptide sequence. Whether this is the case can easily be checked with activity tests for the biological activity, which are known in the prior art or are described in the practical examples. During this, the peptide sequence identity is typically determined with a sequence comparison algorithm. For this, two sequences are compared with one another either over their whole length or over the length of a previously defined segment which makes up at least half of the amino acids of one of the two sequences. Within the comparison window, i.e. the region of the two sequences which is to be compared, the number of identical amino acids at identical or comparable positions is determined. For this, it may be necessary to introduce gaps into a sequence. In the context of the invention, an amino acid sequence should especially preferably be performed with an algorithm known in the prior art, in particular with one of the following algorithms which are made available on the home page of the NCBI: BLASTp, PSI-BLAST, PHI-BLAST or DELTA-BLAST (see also Johnson 2008, Nucleic Acids Res 36 (Web Server issue):W5-9; Boratyn 2012, Biol Direct. 17(7):12; Ye 2012, BMC Bioinformatics 13:134; Ye 2013, Nucleic Acids Res 41: (Web Server issue):W34-40; Marchler-Bauer 2009, Nucleic Acids Res 37 (Database issue):D205-10; and Papadopoulos 2007, Bioinformatics 23(9):1073-9.) Preferably the specified standard settings should be used for this.

In addition, variants of the enzymes to be used according to the invention can preferably be encoded by nucleic acid sequences which are capable of hybridizing under stringent hybridization conditions with the nucleic acid sequences coding for the specific peptide sequences. Stringent hybridization conditions in the sense of the present inventions are described in Southern 1975, J. Mol. Biol. 98(3): 503-517. Here also, it goes without saying that such variants of the enzymes should essentially have the biological activity of the aforesaid enzymes with specific peptide sequence.

According to a further aspect of the invention, a methylotrophic bacterium contains recombinant DNA coding for at least one polypeptide with enzymatic activity for heterologous expression in said bacterium, wherein the polypeptides with enzymatic activity include the following enzymes:
the enzymes of a heterologous mevalonate pathway (MVA pathway), namely hydroxymethylglutaryl-CoA synthase (HMG-CoA synthase), hydroxymethylglutaryl-CoA reductase (HMG-CoA reductase), mevalonate kinase, phosphomevalonate kinase, pyrophosphomevalonate decarboxylase and isopentenyl pyrophosphate isomerase, wherein the enzymes contain a peptide sequence with an identity of respectively at least 60% to the peptide sequence according to SEQ ID No. 1, SEQ ID No. 2, SEQ ID No. 3, SEQ ID No. 4, SEQ ID No. 5 or SEQ ID No. 6 or are encoded by nucleic acid sequences which are capable of hybridizing under stringent hybridization conditions with the corresponding nucleic acids coding for the specific peptide sequences.
a heterologous terpene synthase and
an, in particular heterologous, synthase of a prenyl diphosphate precursor.

According to an advantageous embodiment of the bacterium according to the invention, the enzymes of the heterologous mevalonate pathway, namely hydroxymethylglutaryl-CoA synthase (HMG-CoA synthase), hydroxymethylglutaryl-CoA reductase (HMG-CoA reductase), mevalonate kinase, phosphomevalonate kinase, pyrophosphomevalonate decarboxylase and isopentenyl pyrophosphate isomerase each mutually independently have a peptide sequence with an identity of at least 65%, at least 70%, at least 75%, optionally at least 80%, in particular at least 85%, more particularly at least 90%, preferably at least 95%, more preferably at least 98% and especially preferably at least 99% to the peptide sequence according to SEQ ID No. 1, SEQ ID No. 2, SEQ ID No. 3, SEQ ID No. 4, SEQ ID No. 5 or SEQ ID No. 6.

According to a preferred embodiment of the bacterium according to the invention, the enzymes of the heterologous mevalonate pathway are the hydroxymethylglutaryl-CoA synthase (HMG-CoA synthase), the hydroxymethylglutaryl-CoA reductase (HMG-CoA reductase), the mevalonate kinase, the phosphomevalonate kinase, the pyrophosphomevalonate decarboxylase and the isopentenyl pyrophosphate isomerase from Myxococcus xanthus, respectively having a peptide sequence according to SEQ ID No. 1, SEQ ID No. 2, SEQ ID No. 3, SEQ ID No. 4, SEQ ID No. 5 or SEQ ID No. 6.

According to a further aspect of a bacterium according to the invention, the recombinant DNA, coding for said enzymes of the heterologous mevalonate pathway, comprises the following polynucleotides each mutually independently with an identity of at least 60%, at least 65%, at least 70%, at least 75%, optionally at least 80%, in particular at least 85%, more particularly at least 90%, preferably at least 95%, more preferably at least 98% and especially preferably at least 99% to a nucleotide sequence according to SEQ ID No. 7, SEQ ID No. 8, SEQ ID No. 9, SEQ ID No. 10, SEQ ID No. 11 or SEQ ID No. 12. or polynucleotides which comprise nucleic acid sequences which hybridize under stringent hybridization conditions with a nucleotide sequence according to SEQ ID No. 7, SEQ ID No. 8, SEQ ID No. 9, SEQ ID No. 10, SEQ ID No. 11 or SEQ ID No. 12.

In this, the nucleic acid sequence identity is typically determined with a sequence comparison algorithm. For this, two sequences are compared with one another either over their whole length or over the length of a previously defined segment which makes up at least half of the nucleotides of one of the two sequences. Within the comparison window, i.e. the region of the two sequences which is to be compared, the number of identical nucleotides at identical or comparable positions is determined. For this, it may be necessary to introduce gaps into a sequence. In the context of the invention, an amino acid sequence should especially preferably be carried out with an algorithm known in the prior art, in particular with one of the following algorithms, which are made available on the home page of the NCBI: BLASTn, megablast or discontiguous blast (see Johnson 2008, Nucleic Acids Res. 1;36(Web Server issue):W5-9). Preferably, the specified standard settings should be used in this.

In particular, the polynucleotides used according to the invention are the genes hmgs (SEQ ID No. 7), hmgr (SEQ ID No. 8), mvaK1 (SEQ ID No. 9), mvaK2 (SEQ ID No. 10), mvaD (SEQ ID No. 11) and fni (SEQ ID No.12) from Myxococcus xanthus . The enzymes of the heterologous mevalonate pathway of a bacterium according to this embodiment variant have the peptide sequences according to SEQ ID No. 1, SEQ ID No. 2, SEQ ID No. 3, SEQ ID No. 4, SEQ ID No. 5 or SEQ ID No. 6.

The use of prokaryotic MVA genes, in particular from Myxococcus xanthus, is associated with advantages. The comparable GC content, such as from Myxococcus xanthus of ca. 70%, results in a very good codon adaptation index (Codon Adaptation Indices, CAI), for example between about 0.7 and about 0.9, for the MVA genes.

According to a further aspect of the invention the recombinant DNA coding for said enzymes of the heterologous mevalonate pathway is positioned in one single operon. This enables a better co-regulation of expression with the aid of one single promoter. If required, further heterologous genes can also be integrated into such an operon.

The recombinant DNA coding for said enzymes of the heterologous mevalonate pathway is also referred to synonymously as MVA genes.

According to an advantageous further development, the ribosome binding site (RBS) of at least one of said MVA genes is optimized with regard to the translation initiation for the heterologous expression in the bacterium.

According to an advantageous implementation, the RBS of the gene for the heterologous isopentenyl pyrophosphate isomerase is optimized with regard to translation initiation. Such an RBS-optimized variant of the gene, in particular of the gene fni from Myxococcus xanthus, has a TIR (translation initiation rate according to Salis 2011) of 50 to 200,000, preferably 50 to 100,000, especially preferably 50,000 to 100,000.

According to a further advantageous implementation, the RBS of the gene for the heterologous hydroxymethylglutaryl-CoA synthase (HMG-CoA synthase) is optimized with regard to translation initiation. Such an RBS-optimized variant of the gene, in particular of the gene hmgs from Myxococcus xanthus, has a TIR of 50 to 100,000, preferably 50 to 50,000, especially preferably 1,000 to 50,000.

According to a further preferred embodiment of the invention, the recombinant DNA of the bacterium further codes for at least one heterologous terpene synthase, wherein the terpene synthase is selected from the group consisting of a sesquiterpene synthase and diterpene synthase. It is recognized that the sesquiterpenes and diterpenes formed by said terpene synthases are biotechnologically valuable products.

According to one modification, the at least one heterologous terpene synthase is a sesquiterpene synthase. The sesquiterpene synthase is preferably an enzyme for the synthesis of a cyclic sesquiterpene, wherein the sesquiterpenes are in particular selected from the group consisting of α-humulene, various epimers of santalene, such as α-santalene, β-santalene, epi-β-santalene or α-exo-bergamotene, and bisabolenes, such as β-bisabolene.

The sesquiterpene synthase is more preferably an α-humulene synthase or a santalene synthase. Here it should be noted that in particular the santalene synthase has a very broad product spectrum and thus a great multiplicity of different sesquiterpenes of the santalene type are obtainable.

The sesquiterpene synthase is preferably a sesquiterpene synthase of plant origin. Preferably the sesquiterpene synthase is an enzyme from an organism, wherein the organism is selected from the group consisting of the genus Zingiberand Santalum. Sesquiterpene synthases from other organisms with appropriate suitability can also be used.

The sesquiterpene synthase according to a further aspect comprises a peptide sequence with an identity of at least 60% to a polypeptide selected from the group consisting of a polypeptide of the peptide sequence according to SEQ ID No. 15, a polypeptide of the peptide sequence according to SEQ ID No. 45 and a polypeptide of the peptide sequence according to SEQ ID No. 46.

Sesquiterpene synthases in the sense of the invention can also be enzymes with appropriate activity which are encoded by polynucleotides which comprise nucleic acid sequences which hybridize under stringent hybridization conditions with a nucleotide sequence which encodes one of the polypeptides according to SEQ ID No.: 15, 45 or 46.

Said peptide sequence of a sesquiterpene synthase more preferably has an identity of at least 65%, at least 70%, at least 75%, optionally at least 80%, in particular at least 85%, more particularly at least 90%, preferably at least 95%, more preferably at least 98% and especially preferably at least 99%, to a polypeptide selected from the group consisting of a polypeptide of the peptide sequence according to SEQ ID No. 15, a polypeptide of the peptide sequence according to SEQ ID No. 45 and a polypeptide of the peptide sequence according to SEQ ID No. 46.

Preferably the sesquiterpene synthase is an enzyme containing a polypeptide with appropriate activity from Zingiber zerumbet, Santalum album or Santalum spicatum.

The sesquiterpene synthase is in particular the α-humulene synthase from Zingiber zerumbet, which contains a polypeptide according to the peptide sequence according to SEQ ID No. 15. According to a further development, the α-humulene synthase which contains a polypeptide according to the peptide sequence according to SEQ ID No. 15 is encoded by a recombinant DNA comprising a polynucleotide with a nucleic acid sequence according to SEQ ID No. 16. Said nucleic acid sequence according to SEQ ID No. 16 is the gene zssl from Zingiber zerumbet, which was codon-optimized for expression in Methylobacterium extorquens AM1.

According to an alternative implementation, the sesquiterpene synthase is in particular the santalene synthase SsaSSy from Santalum album, which contains a polypeptide according to the peptide sequence according to SEQ ID No. 45.

According to a further alternative implementation, the sesquiterpene synthase is preferably the santalene synthase SspiSSy from Santalum spicatum, which contains a polypeptide according to the peptide sequence according to SEQ ID No. 46.

According to an alternative modification, the at least one heterologous terpene synthase is a diterpene synthase. The diterpene synthase is preferably an enzyme for the synthesis of a diterpene, wherein the diterpene is selected from the group consisting of sclareol, cis-abienol, abitadiene, isopimaradiene, manool and larixol. Preferably the diterpene synthase of plant origin is in particular from the genera Salvia or Abies.

According to a further aspect, the diterpene synthase comprises a peptide sequence with an identity of at least 40% to a polypeptide of the peptide sequence according to SEQ ID No. 47.

Diterpene synthases in the sense of the invention can also be enzymes with appropriate activity which are encoded by polynucleotides which comprise nucleic acid sequences which hybridize under stringent hybridization conditions with a nucleotide sequence which encodes the polypeptide according to SEQ ID No. 47.

Said peptide sequence of a diterpene synthase, more preferably possesses an identity of at least 45%, at least 50%, at least 55%, at least 60%, at least 65%, at least 70%, at least 75%, optionally at least 80%, in particular at least 85%, more particularly at least 90%, preferably at least 95%, more preferably at least 98% and especially preferably at least 99%, to a polypeptide of the peptide sequence according to SEQ ID No. 47.

The diterpene synthase is further preferably selected from the group consisting of the two monofunctional type I and type II diterpene synthases SsLPS, the Salvia sclarea LPP synthase and SsSCS, the S. sclarea sclareol synthase (Caniard et al., BMC Plant Biol 2012 Jul 26;12:119), which are co-expressed, the bifunctional type I/type II diterpene synthase cis-abienol synthase AbCAS, from Abies balsamea (Zerbe et al., J Biol Chem 2012 Apr 6;287(15):12121-31), the LPP synthase NtCPS2 and the cis-abienol synthase NtABS from Nicotiana tabacum (Sallaud et al., Plant J 2012 Oct;72(1):1-17).

According to one aspect, the diterpene synthase is in particular the bifunctional type I/type II diterpene synthase cis-abienol synthase AbCAS from Abies balsamea, which contains a polypeptide according to the peptide sequence according to SEQ ID No. 47.

Especially preferably, the cis-abienol synthase is encoded by a polynucleotide codon-optimized for M. extorquens AM1, quite especially preferably by a polynucleotide which has a sequence with SEQ ID No.: 50.

As already stated above, the prenyl diphosphate precursors—such as FPP or GGPP—form the respective substrates of the terpene synthases. Thus those skilled in the art recognize that during selection of a certain terpene synthase, the suitable synthase for the provision of the appropriate prenyl diphosphate precursor must be selected.

According to an advantageous further development, the RBS of the gene for the sesquiterpene synthase is optimized with regard to translation initiation. Such an RBS-optimized variant of the gene has a TIR (translation initiation rate) of at least 50,000, in particular 50,000 to 400,000, preferably from 200,000 to 300,000, especially preferably 210,000 to 250,000.

The bacterium according to a further embodiment, in addition to the recombinant DNA coding for at least one enzyme of a heterologous mevalonate pathway, and further if required has recombinant DNA coding for at least one synthase of a prenyl diphosphate precursor.

The synthase of a prenyl diphosphate precursor is either an endogenous or a heterologous enzyme. In the case of an endogenous synthase of a prenyl diphosphate precursor, the gene coding for it is preferably overexpressible with the aid of a suitable promoter.

According to a preferred implementation, the bacterium in addition to the recombinant DNA coding for at least one enzyme of a heterologous mevalonate pathway further contains recombinant DNA coding for at least one heterologous synthase of a prenyl diphosphate precursor. If required, a heterologous synthase of a prenyl diphosphate precursor can be expressible in addition to a corresponding endogenous enzyme.

The synthase of the prenyl diphosphate precursor is an enzyme selected from the group consisting of farnesyl diphosphate synthase (FPP synthase) and geranylgeranyl diphosphate-synthase (GGPP synthase). It is recognized that the prenyl diphosphate precursors, FPP and GGPP respectively formed from said synthases are important precursor molecules for a synthesis of biotechnologically valuable sesquiterpenes and diterpenes.

According to one modification, the synthase of a prenyl diphosphate precursor is a heterologous FPP synthase, where this can be a eukaryotic or prokaryotic heterologous FPP synthase. The heterologous FPP synthase can for example be of bacterial origin or derive from a fungus.

The heterologous FPP synthase is in particular an enzyme from a fungus, preferably from a yeast, such as of the genus Saccharomyces.

According to a further aspect, the FPP synthase comprises a peptide sequence with an identity of at least 60% to the peptide sequence according to SEQ ID No. 13. The FPP synthase is in particular a eukaryotic FPP synthase.

FPP synthases in the sense of the invention can also be enzymes with appropriate activity, which are encoded by polynucleotides which comprise nucleic acid sequences which hybridize under stringent hybridization conditions with a nucleotide sequence which codes for the polypeptide according to SEQ ID No.: 13.

According to a further implementation, said peptide sequence of a FPP synthase preferably possesses an identity of at least 65%, at least 70%, at least 75%, optionally at least 80%, in particular at least 85%, more particularly at least 90%, preferably at least 95%, more preferably at least 98% and especially preferably at least 99%, to SEQ ID No. 13.

According to a further aspect of a bacterium according to the invention, the recombinant DNA, coding for the FPP synthase comprises a polynucleotide with an identity of at least 60%, at least 65%, at least 70%, at least 75%, optionally at least 80%, in particular at least 85%, more particularly at least 90%, preferably at least 95%, more preferably at least 98% and especially preferably at least 99% to a nucleotide sequence according to SEQ ID No. 14.

The FPP synthase is preferably a FPP synthase from Saccharomyces cerevisiae. FPP synthases from other organisms can with appropriate suitability also be used. The FPP synthase is in particular the FPP synthase ERG20 from Saccharomyces cerevisiae which contains a polypeptide according to SEQ ID No. 13.

According to one implementation, the FPP synthase ERG20 from Saccharomyces cerevisiae having a polypeptide according to SEQ ID No. 13 is in particular encoded by a polynucleotide with a sequence according to SEQ ID No. 14.

According to a further modification, the synthase of a prenyl diphosphate precursor is a heterologous geranylgeranyl diphosphate synthase (GGPP synthase). The heterologous GGPP synthase is in particular an appropriate enzyme from a bacterium, a plant or a fungus. Preferably the GGPP synthase is an enzyme from an organism, where the organism is preferably selected from the group consisting of a bacterium of the family of the Enterobacteriaceae, a plant of the genus Taxus and a fungus of the genus Saccharomyces. Suitable GGPP synthases from bacteria of the family of the Enterobacteriaceae can for example be appropriate enzymes from bacteria of the genus Pantoea.

According to a further aspect, the GGPP synthase comprises a peptide sequence with an identity of at least 60% to a polypeptide selected from the group consisting of a polypeptide of the peptide sequence according to SEQ ID No. 43, a polypeptide of the peptide sequence according to SEQ ID No. 44 and a polypeptide of the peptide sequence according to SEQ ID No. 42.

GGPP synthases in the sense of the invention can also be enzymes with appropriate activity which are encoded by polynucleotides which comprise nucleic acid sequences which hybridize under stringent hybridization conditions with a nucleotide sequence, which encodes one of the polypeptides according to SEQ ID No.: 43, 44 or 42.

Said peptide sequence of a GGPP synthase more preferably possesses an identity of at least 65%, at least 70%, at least 75%, optionally at least 80%, in particular at least 85%, more particularly at least 90%, preferably at least 95%, more preferably at least 98% and especially preferably at least 99%, to a polypeptide selected from the group consisting of a polypeptide of the peptide sequence according to SEQ ID No. 43, a polypeptide of the peptide sequence according to SEQ ID No. 44 and a polypeptide of the peptide sequence according to SEQ ID No. 42.

The GGPP synthase is preferably an enzyme having a polypeptide with appropriate activity from Pantoea agglomerans or Pantoea ananatis, Taxus canadensis or Saccharomyces cerevisiae.

According to a further aspect, the GGPP synthase is an enzyme selected from the group consisting of the GGPP synthase crtE from Pantoea agglomerans having a peptide sequence according to SEQ ID No. 43, the GGPP synthase from Taxus canadensis having a peptide sequence according to SEQ ID No. 44 and the GGPP synthase BTS1 from Saccharomyces cerevisiae having a peptide sequence according to SEQ ID No. 42. GGPP synthases from other organisms with appropriate suitability can also be used.

According to an advantageous further development, the RBS of the recombinant DNA, i.e. of the gene for the heterologous synthase of a prenyl diphosphate precursor, such as FPP or GGPP synthase, is optimized with regard to translation initiation. Such an RBS-optimized variant of the gene has a TIR (translation initiation rate) of 500 to 100,000, preferably of 10,000 to 50,000, especially preferably 20,000 to 40,000.

According to an advantageous implementation of the invention, the RBS for the genes coding for the heterologous terpene synthase and the heterologous synthase of a prenyl diphosphate precursor are adapted to the extent that the TIR value for the heterologous terpene synthase is higher than the TIR value for the heterologous synthase of a prenyl diphosphate precursor. Accumulation of prenyl diphosphate precursors, sometimes with toxic effects, can thus be avoided.

If necessary, the recombinant DNA is codon-optimized for expression in the bacterium according to the invention. For example, the gene coding for the heterologous terpene synthase is codon-optimized for the bacterium according to the invention. In this way, the expression in the methylotrophic bacterium can be improved.

According to a further embodiment of the bacterium, the recombinant DNA codes for the FPP synthase, the ERG20 FPP synthase from Saccharomyces cerevisiae and the recombinant DNA codes for the sesquiterpene synthase, the α-humulene synthase from Zingiber zerumbet.

The bacterium according to one of the implementations is preferably further developed to the effect that the recombinant DNA for heterologous expression of said enzymes is provided with a common promoter or several mutually independently inducible promoters. This can be differently configured for the respective genes. Here the inducible promoters can be different in nature, so that they are mutually independently regulatable. Preferably, all genes for expression of the enzymes mentioned here are provided with the same common inducible promoter.

Inducible promoter systems are in principle known to those skilled in the art. Preferably a very “tight” promoter system is utilized here. In this manner, the expression of the recombinant genes can be deliberately switched on at a desired time point in the culturing. In particular, for the regulation of expression of the MVA pathway genes, a particularly “tight” promoter system is of advantage, since otherwise growth-influencing effects can arise. Particularly preferable is a cumate-inducible system.

According to an advantageous embodiment of the bacterium, the recombinant DNA is in each case expressible on plasmid or chromosomally. This can also be differently configured for the respective genes. Suitable chromosomal sites and techniques for stable integration into the genome are known to those skilled in the art. For the purpose of plasmid-located expression, suitable plasmids with the recombinant DNA are introduced into the bacterium by transformation. The bacterium according to the invention is thus preferably obtained by transformation with one or more plasmid(s), which bears or bear the relevant recombinant DNA.

According to a preferred embodiment, the bacterium according to the invention contains at least one plasmid introduced by transformation, wherein the at least one plasmid comprises the following recombinant DNA:
recombinant DNA coding for at least one enzyme of a heterologous mevalonate pathway as mentioned above, wherein the enzyme of the mevalonate pathway is selected from the group consisting of hydroxymethylglutaryl-CoA synthase (HMG-CoA synthase), hydroxymethylglutaryl-CoA reductase (HMG-CoA reductase), mevalonate kinase, phosphomevalonate kinase, pyrophosphomevalonate decarboxylase and isopentenyl pyrophosphate isomerase;
optionally recombinant DNA coding for at least one heterologous synthase of a prenyl diphosphate precursor as mentioned above; and
recombinant DNA coding for at least one heterologous terpene synthase as mentioned above.

The methylotrophic bacterium in the sense of the present invention is in particular a proteobacterium. A preferred methylotrophic proteobacterium is selected from the genera Methylobacterium and Methylomonas . More preferably, the methylotrophic proteobacterium is a strain of the genus Methylobacterium , in particular a strain of Methylobacterium extorquens . Especially preferred is the strain Methylobacterium extorquens AM1 or the strain Methylobacterium extorquens PAI.

Furthermore, a strain of the methylotrophic bacterium, in particular of the genera Methylobacterium and Methylomonas , preferably of Methylobacterium extorquens AM1 or PA1, lacking carotenoid biosynthesis activity, preferably with a defect in the gene crtNb (diapolycopene oxidase) (Van Dien et al., 2003, Appl Environ Microbiol 69, 7563-6.) is preferred. Such a strain has no carotenoid biosynthesis activity, in particular diapolycopene oxidase activity is lacking. A further improvement in the terpene synthesis rate is thereby possible.

A further aspect of the present invention relates to a method for de novo microbial synthesis of sesquiterpenes or diterpenes from methanol and/or ethanol, comprising the following steps:
preparing a methanol and/or ethanol-containing aqueous medium,
culturing a methylotrophic bacterium as described in one of the embodiments presented above in said medium in a bioreactor, wherein methanol and/or ethanol is converted into a terpene by the bacterium,
separating the sesquiterpene or diterpene formed in the bioreactor.

The aqueous medium used can contain methanol, ethanol or a mixture of methanol and ethanol. If required, further substrates can be added thereto. It can be advantageous that exclusively methanol or ethanol is contained in the aqueous medium, i.e. the de novo microbial synthesis of sesquiterpenes or diterpenes takes place either from methanol or from ethanol.

The use of methanol brings many advantages: methanol can be produced both petrochemically and also from renewable raw materials or in the future even from CO_{2.} There are neither seasonal (weather and time of year) nor regional factors, which makes long-term production planning possible. Apart from this, it is probable that in contrast to that of sugar, the price of methanol will sink in the future, because of many production plants planned or under construction. Methanol is a carbon source in alternative to the otherwise commonly used sugar substrates.

In methanol, as also in ethanol, the carbon has a lower oxidation level in comparison to carbohydrates/sugars. As a result, in the oxidation of methanol to CO₂ more electrons can be released than in the case of the complete oxidation of sugar to CO_{2.} For the synthesis of strongly reduced compounds such as terpenes it is therefore advantageous as far as possible to use carbon sources which have a low oxidation level. Methanol and ethanol fulfil these preconditions better than carbon from sugars. Hence the use of methanol/ethanol is more advantageous for the production of terpenes than starting from carbohydrates.

The use of ethanol also brings many advantages: ethanol is available as a “natural” substrate, for example from biomass fermentation, i.e. bio-ethanol.

Furthermore, the use of bio-ethanol for de novo microbial synthesis of sesquiterpenes or diterpenes in particular enables an advantageous declaration of the sesquiterpene and diterpene products formed as “natural aroma substances”. Ethanol is an alternative carbon source to the otherwise commonly used sugar substrates.

The bacteria according to the invention grow as required both on methanol and also on ethanol, in each case as the sole carbon source. In a further development of the method, methanol and/or ethanol is contained in said medium as the sole carbon source for culturing said bacterium. Thereby it is in particular understood that no further carbon source is deliberately added to the medium or contained in major proportions. It is evident that traces of further carbon sources are not always avoidable, and may be contained without departing from the scope of said further development of the method according to the invention.

According to an advantageous embodiment of the method, a methanol and/or ethanol-limited fed batch fermentation is performed.

In the sense of a preferred implementation of the method, an in situ removal of the sesquiterpene or diterpene from the bioreactor takes place, i.e. in particular an in situ product removal (ISPR) in the fermenter. An important aspect of industrial biotechnology, apart from the actual product synthesis is also its workup. In situ product-removal (ISPR) reduces both the toxic effects of the product on the microorganism and also the costs of the method. The ISPR is effected here in particular by stripping of the terpene. In this, the terpene is preferably transferred into the exhaust gas stream and then dissolved in an organic solvent. It is particularly advantageous that because of the reduced substrate methanol or ethanol in contrast to conventional microorganisms, which grow with sugar, said methylotrophic bacteria are cultured at markedly higher aeration rates and at the same time the stripping of volatile fermentation products, in particular the sesquiterpene or diterpene formed, is considerably favored thereby.

In a further development of the method, the culturing takes place in an aqueous organic two phase system, wherein the organic phase in particular is constituted by an aliphatic hydrocarbon compound, in particular an alkane, preferably dodecane or decane. The terpenes formed have good solubility in said organic phase.

According to an advantageous embodiment of the method, the culturing is performed at essentially constant pH. In particular, the method is performed with a dissolved oxygen level of >30% and/or a methanol or ethanol concentration of about 1 g/L,

In a further development of the method, a terpene concentration of more than 0.75, 0.8, 0.9, preferably more than 1.0 g/I, more preferably more than 1.5 g/I, each based on the volume of the aqueous phase, is reached.

A further aspect of the present invention relates to the use of a methanol or ethanol-containing medium for culturing a recombinant methylotrophic bacterium as described in one of the embodiments described above for the de novo microbial synthesis of terpenes from methanol and/or ethanol. The use of a methanol or ethanol minimal medium reduces both the contamination risk, since methanol and ethanol are toxic or growth-inhibiting for many microorganisms, and also the cost during the product workup, since no complex components have to be removed from the actual product.

Numerous facts show the advantages of the use of methanol and/or ethanol compared to sugars: i) sugar is not only glucose, the purification thereof is regularly necessary to guarantee the yield, wherein an evaluated purification is associated with costs, ii) a rise in the sugar prices in the coming years is to be expected, whereas the methanol and ethanol prices will probably sink because of considerably increased production capacities, iii) methanol and ethanol considerably reduce contamination risks compared to sugar-based fermentations, which reduces the sterilization cost, and iv) methanol or ethanol minimal medium contains no complex chemical compounds in contrast to medium with glucose or other sugar sources (such as corn steep liquor or lignocellulose) as carbon source, which simplifies purification methods.

A suitable fermentation medium can for example have the following composition: water, methanol or ethanol and further components selected from the group consisting of PIPES, NaH₂PO₄, K₂HPO₄, MgCl₂, (NH₄)₂SO₄, CaCl₂, sodium citrate, ZnSO₄, MnCl₂, FeSO₄, (NH₄)₆Mo₇O₂₄, CuSO₄ and CoCl₂.

A further increase in the terpene formation can be achieved by blocking the carotenoid synthesis in the proteobacterium used. For this, said strains of the genus Methylobacterium or of the genus Methylomonas lacking carotenoid biosynthesis activity, in particular lacking diapolycopene oxidase activity, are advantageously used. A maximum terpene concentration of over 1.5 g/I, in particular of about 1.65 g/I, each based on the volume of the aqueous phase, can thus be achieved.

The already mentioned mutant according to the invention of Methylobacterium extorquens AM1 lacking carotenoid biosynthesis activity, in particular lacking diapolycopene oxidase activity, exhibits increased terpene production. In particular, a maximum α-humulene concentration of over 1.5 g/I, in particular about 1.65 g/I, was formed by a mutant of Methylobacterium extorquens AM1 lacking carotenoid biosynthesis activity.

It is noteworthy here that the aforesaid concentrations of terpenes are already reached according to the invention without for example expensive lithium acetoacetate or DL-mevalonate having to be externally added. Furthermore, in particular no further costly measures for strain optimization are absolutely necessary for this. The considerable potential of the methylotrophic bacteria and of said method for the biotechnological production of terpenes already follows from this. It is moreover advantageous that the aforesaid concentrations are already reached with use of inexpensive methanol or ethanol minimal medium. In contrast to the prior art, no fermentation medium based on TB or LB is necessary. A further advantage emerges therefrom in the simplification of the purification of the terpene products obtained, since a clearly defined minimal medium can be used. Costly removal of by-products can be minimized. In addition, the strains described here open up the use of methanol or ethanol as the sole carbon source for growth.

A further aspect of the present invention relates to the use of a methylotrophic bacterium as described in one of the embodiments described above for the de novo microbial synthesis of terpenes from methanol and/or ethanol.

The terpenes formed according to the method according to the invention are selected from the group consisting of sesquiterpenes (C15) and diterpenes (C20).

Terpenes in the sense of the method according to the invention are on the one hand sesquiterpenes. The biotechnologically interesting sesquiterpenes accordingly include for example sesquiterpenes selected from the group consisting of α-humulene, various epimers of santalene such as α-santalene, β-santalene, epi-3-santalene and α-exo-bergamotene. Bisabolenes, such as 6-bisabolene, are also sesquiterpenes which are obtainable by the method according to the invention. Suitable sesquiterpene synthases are in principle known to those skilled in the art. The aforesaid methylotrophic bacteria can thus optionally be equipped with the appropriate recombinant genes coding for the suitable sesquiterpene synthases. In particular, for this the methylotrophic bacterium as well as the heterologously expressed genes of the MVA pathway also contains an FPP synthase in the aforesaid sense.

Terpenes in the sense of the method according to the invention are on the other hand diterpenes. The biotechnologically interesting diterpenes accordingly include for example diterpenes selected from the group consisting of sciareol, cis-abienol, abitadiene, isopimaradiene, manool and larixol. Suitable diterpene synthases are in principle known to those skilled in the art. The aforesaid methylotrophic bacteria can thus optionally be equipped with the appropriate recombinant genes coding for the suitable diterpene synthases. In particular, for this the methylotrophic bacterium as well as the heterologously expressed genes of the MVA pathway also contain a GGPP synthase in the aforesaid sense.

According to an especially preferable implementation of the method according to the invention, the sesquiterpene α-humulene of the formula I

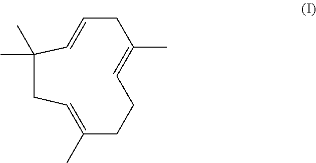

is synthesized de novo from methanol and/or ethanol,

According to further preferred implementations of the method according to the invention, sesquiterpene of the santalene type selected from the group consisting of α-santalene of the formula II, β-santalene of the formula III, epi-β-santalene of the formula IV, and α-exo-bergamotene of the formula V

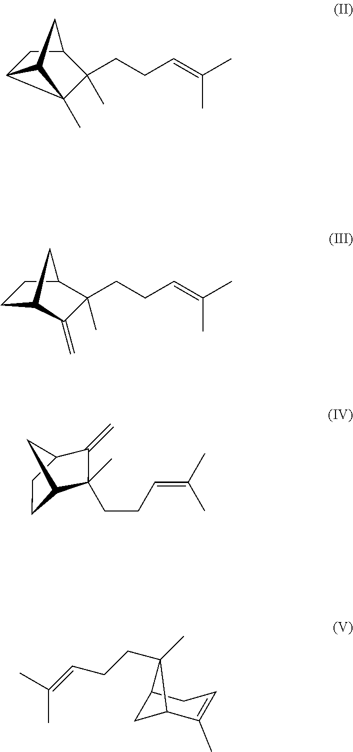

are synthesized de novo from methanol and/or ethanol. Here it should be noted that the santalene synthase possesses a very broad product spectrum and thus a great multiplicity of different sesquiterpenes of the santalene type is obtainable.

According to further preferred embodiments of the method according to the invention, the diterpenes sciareol of the formula VI and cis-abienol of the formula VII

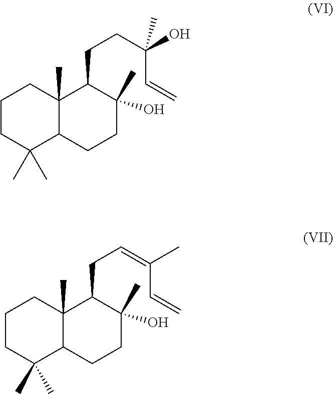

are synthesized de novo from methanol and/or ethanol.

A bioreactor in the sense of the present invention can be any suitable vessel for culturing bacteria. In the simplest case, this is understood to mean a shaker flask. In particular it is understood to mean a fermenter. The bioreactor can be suitable for continuous operation, discontinuous operation, fed batch operation or batch production.

A further aspect of the present invention relates to said sesquiterpenes (C15) and diterpenes (C20) obtainable by a method according to one of the implementations presented.

Below, further embodiments of the bacteria, methods and uses according to the invention are described:

1. A methylotrophic bacterium containing recombinant DNA coding for at least one polypeptide with enzymatic activity for expression in said bacterium, characterized in that said at least one polypeptide with enzymatic activity is selected from the group consisting of
at least one enzyme of a heterologous mevalonate pathway selected from the group consisting of hydroxymethylglutaryl-CoA synthase (HMG-CoA synthase), hydroxymethylglutaryl-CoA reductase (HMG-CoA reductase), mevalonate kinase, phosphomevalonate kinase, pyrophosphomevalonate decarboxylase and isopentenyl pyrophosphate isomerase;
a heterologous terpene synthase and
optionally a synthase of a prenyl diphosphate precursor.

2. The bacterium as described in embodiment 1, characterized in that the at least one enzyme of the heterologous mevalonate pathway contains a peptide sequence with an identity of respectively at least 60% to the peptide sequence according to SEQ ID No. 1, SEQ ID No. 2, SEQ ID No. 3, SEQ ID No. 4, SEQ ID No. 5 or SEQ ID No. 6.

3. The bacterium as described in embodiment 1 or 2, characterized in that the heterologous terpene synthase is selected from the group consisting of a sesquiterpene synthase and a diterpene synthase.

4. The bacterium as described in embodiment 3, characterized in that the heterologous terpene synthase is a sesquiterpene synthase, wherein the sesquiterpene synthase is an enzyme for the synthesis of a cyclic sesquiterpene, and the sesquiterpene is in particular selected from the group consisting of α-humulene and epimers of santalene, such as α-santalene, β-santalene, epi-β-santalene or α-exo-bergamotene, and bisabolenes, such as b-bisabolene.

5. The bacterium as described in embodiment 3, characterized in that the heterologous terpene synthase is a diterpene synthase, in particular an enzyme for the synthesis of a diterpene, and the diterpene is in particular selected from the group consisting of sclareol, cis-abienol, abitadiene, isopimaradiene, manool and Iarixol.

6. The bacterium as described in one of embodiments 1 to 5, characterized in that the synthase of a prenyl diphosphate precursor is an enzyme selected from the group consisting of farnesyl diphosphate synthase (FPP synthase) and geranylgeranyl diphosphate synthase (GGPP synthase).

7. The bacterium as described in one of embodiments 1 to 6, characterized in that the synthase of a prenyl diphosphate precursor is a heterologous FPP synthase, wherein the heterologous FPP synthase is a eukaryotic or prokaryotic FPP synthase.

8. The bacterium as described in one of embodiments 1 to 6, characterized in that the synthase of a prenyl diphosphate precursor is a heterologous GGPP synthase, wherein the heterologous GGPP synthase is an enzyme from an organism which is selected from the group consisting of bacteria, plants and fungi.

9. The bacterium as described in one of embodiments 1 to 8, characterized in that the recombinant DNA for heterologous expression of said enzymes is provided with a common inducible promoter or several mutually independently inducible promoters.

10. The bacterium as described in one of embodiments 1 to 9, characterized in that the recombinant DNA is in each case mutually independently expressible on plasmid or chromosomally.

11. The bacterium as described in one of embodiments 1 to 10, characterized in that the bacterium is a methylotrophic proteobacterium, in particular a bacterium of the genus Methylobacterium or of the genus Methylomonas , preferably the bacterium Methylobacterium extorquens.

12. The bacterium as described in one of embodiments 1 to 11, characterized in that the bacterium is a strain lacking carotenoid biosynthesis activity, in particular lacking diapolycopene oxidase activity.

13. A method for de novo microbial synthesis of sesquiterpenes or diterpenes from methanol and/or ethanol, comprising the following steps:
preparing a methanol and/or ethanol-containing aqueous medium,
culturing a methylotrophic bacterium as described in one of the embodiments 1 to 12 in said medium in a bioreactor, whereby methanol and/or ethanol is converted into a terpene by the bacterium,
separating the sesquiterpene or diterpene formed in the bioreactor.

14. The method as described in embodiment 13, characterized in that in said medium methanol and/or ethanol is/are contained as the sole carbon source(s) for culturing said bacterium.

15. The method as described in embodiment 13 or 14, characterized in that a methanol and/or ethanol-limited fed batch fermentation is performed.

16. The method as described in one of embodiments 13 to 15, characterized in that the culturing takes place in an aqueous organic two phase system, wherein the organic phase in particular is constituted by an aliphatic hydrocarbon compound, preferably dodecane or decane.

17. The method as described in one of embodiments 13 to 16, characterized in that an in situ removal of the sesquiterpene or diterpene from the bioreactor is effected, i.e. an in situ product removal (ISPR).

18. The method as described in one of embodiments 13 to 17, characterized in that the culturing is performed at essentially constant pH, dissolved oxygen level of >30% and/or methanol or ethanol concentrations of about 1 g/L.

19. The method as described in one of embodiments 13 to 18, characterized in that a terpene concentration of more than 1 g/I, preferably more than 1.5 g/I, each based on the volume of the aqueous phase, is reached.

20. Use of a methanol and/or ethanol-containing medium for culturing a recombinant methylotrophic bacterium as described in one of embodiments 1 to 12 for the de novo microbial synthesis of sesquiterpenes or diterpenes from methanol and/or ethanol.

21. Use of a methylotrophic bacterium as described in one of embodiments 1 to 12 for the de novo microbial synthesis of sesquiterpenes or diterpenes from methanol and/or ethanol.

The invention is not limited to one of the embodiments described above, but is modifiable in a great variety of ways. Those skilled in the art recognize that the embodiments according to the invention, in particular the bacterial strains and fermentation conditions described, can easily be adapted without departing from the scope of the invention. Thus simple adaptations are conceivable for the production of any sesquiterpenes from methanol or ethanol. The invention enables the bioproduction of terpenes from the carbon source methanol or ethanol not competing with foods. Further characteristics, details and advantages of the invention follow from the wording of the claims and from the following description of practical examples on the basis of the drawings

The content of all literature references cited in this patent application is hereby included by reference to the respective specific disclosure content and in its entirety.

### FIGURES

FIG. 1 shows a schematic overview of the central metabolism of Methylobacterium extorquens AM1 including the endogenous terpene synthesis via the desoxyxylulose-5-phosphate pathway (DXP), the heterologously integrated mevalonate pathway (indicated by two boxes), a heterologous α-humulene synthase zssl and a heterologous FPP synthase ERG20. M. extorquens possesses no IPP isomerase (fni). The heterologously integrated MVA genes relate to a hydroxymethylglutaryl-CoA synthase (hmgs), hydroxymethylglutaryl-CoA reductase (hmgr), mevalonate kinase (mvaK), phosphomevalonate kinase (mvaK2), pyrophosphomevalonate decarboxylase (mvaD) and isopentenyl pyrophosphate isomerase (fni). Further genes: dxs: 1-desoxy-D-xylulose-5-phosphate synthase, dxr: 1-desoxy-D-xylulose-5-phosphate reductase, hrd: HMB-PP reductase, ispA: endogenous FPP synthase; molecule abbreviations: 2PG: 2-phosphoglycerate, 3PG: 3-phosphoglycerate, 1,3-DPG: 1,3-bisphosphoglycerate, GA3P: glyceraldehyde-3-phosphate, PEP: phosphoenol pyruvate, HMG-CoA: hydroxymethylglutaryl-CoA, DXP: 1-desoxy-D-xylulose-5-phosphate, MEP: 2-C-methyl-D-erythritol-4-phosphate, HMB-PP: (E)-4-hydroxy-3-methyl-but-2-enyl pyrophosphate, IPP: isopentenyl pyrophosphate, GPP: geranyl pyrophosphate, FPP: farnesyl pyrophosphate.

FIG. 2 shows a chromatographic comparison of α-humulene standard (upper panel, black line) and a sample from M. extorquens containing pFS33 (pCM80-zssl, upper panel, light gray line). The internal standard zerumbone elutes after 11.5 minutes. α-Humulene in the pFS33 sample was identified by comparison of the mass spectra shown under the chromatogram.

FIG. 3 shows the tolerance of Methylobacterium extorquens AM1 towards α-humulene directly dissolved in the aqueous phase or dissolved in the dodecane phase as a second organic phase. Maximum growth rates in respective medium without α-humulene (μₘₐₓ) are compared with growth rates (μ) at different α-humulene concentrations. It can be seen that α-humulene has only minimal growth-inhibiting effects on M. extorquens, even at concentrations of 1 g/I., α-humulene in the dodecane phase has a slightly lesser influence than in the aqueous phase, since it has less contact with the cells.

FIG. 4 shows the α-humulene production of M. extorquens AM1 bearing the plasmids pFS33 (pCM80-zssl), pFS34 (pCM80-zssl-ERG20), pFS45 (pHC115-zssl), pFS46 (pHC115-zssl-ERG20), pFS49 (pQ2148F-zssl) and pFS50 (pQ2148F-zssl-ERG20). Black bar sections show the production without induction, whereas the gray bar sections represent the production with induction. pCM80 bears a constitutive promoter. The concentrations were compared 48 hours after culturing (pFS33, 34) and after induction (pFS45, 46, 49 50) respectively;

FIG. 5 shows the α-humulene production of M. extorquens bearing the plasmids with optimized ribosome binding sites (RBS) for α-humulene synthase (zssl), FPP synthase (ERG20) and IPP isomerase (fni) in various combinations. The translation initiation rates for the genes are stated on the y-axis in brackets. The concentrations are average product concentrations from three 3 transformants, wherein each was grown in two separate cultures. Black bars: plasmids (pFS49, pFS57) which contain only zssl, hatched bars: plasmids (pFS50, pFS58, pFS60a, pFS60b) which contain zssl and ERG20, gray bars: plasmids (pFS61b, pFS62a, pFS62b) containing zssl, ERG20 and the six genes of the mevalonate pathway.

FIG. 6 A: Chromatograms (n=502 nm) of unsaponified carotenoid extract from E. coli expressing the diapophytoene synthase and diapophytoene desaturase from S. aureus via pACCRT-MN (A1) and from M. extorquens carotenoid biosynthesis deficient strain CM502 (A2). The theoretical retention time of lycopene is indicated with the arrow. B: α-humulene production of M. extorquens AM1 and CM502 bearing plasmid pFS62b (pQ2148F-zssl^{225k}-ERG20^{22k}-fni^{65k}-MVA) in shaker flasks 48 hours after induction (n=3).

FIG. 7: Cell dry weight and α-humulene concentration formed from the strain CM502 bearing pFS62b in fermentation 5 (according to Table 3). The time point 0 gives the time point of induction with cumate, represented by the dotted vertical line. Standard deviations of the α-humulene concentrations were determined from the same sample by threefold analysis. Black squares: α-humulene concentration, gray circles: cell dry weight.

FIG. 8 shows the chromatographic comparison of cis-abienol standard (upper panel, labeled line) and a sample from M. extorquens containing ppjo16 (pQ2148F-AbCAS-ERG20F96C-MVA, upper panel, labeled line). The internal standard zerumbone elutes after 11.3 minutes. Cis-abienol in the 16s6 sample was identified by comparison of the mass spectra shown below the chromatogram.

FIG. 9 shows a chromatographic comparison of sandalwood oil (upper panel (a), dark gray line) and a sample from M. extorquens containing ppjo03 (pQ2148F-SanSyn-ERG20-MVA), upper panel (a), black line). α-Santalene in the ppjo03 sample was identified by comparison of the mass spectra (b, c) shown under the chromatogram.

### EXAMPLES

The following examples serve to illustrate the invention. They must not be interpreted as limiting with regard to the scope of protection.

### Example 1

### Recombinant α-Humulene Production

1. Material and Methods

1.1 Chemicals, Media and Bacterial Strains

Methylobacterium extorquens AM1 (Peel and Quayle, 1961. Biochem J. 81, 465-9) was cultured at 30° C. in minimal media, wherein for the culturing in the shaker flask the medium according to Kiefer et al., 2009 (PLoS ONE. 4, e7831) was used. The fermentation medium contains an end concentration of 30 mM PIPES, 1.45 mM NaH₂PO₄, 1.88 mM K₂HPO₄, 1.5 mM MgCl₂, 11.36 mM (NH₄)₂SO₄, 20 μM CaCl₂, 45.6 μM sodium citrate (Na₃C₆H₅O₇*2H₂O), 8.7 μM ZnSO₄*7H₂O, 15.2 μM MnCl₂*4H₂O, 36 μM FeSO₄*7H₂O, 1 μM (NH₄)₆Mo₇O₂₄*4H₂O, 0.3 μM CuSO₄*5H₂O and 12.6 μM CoCl₂*6H₂O.

Escherichia coil strain DH5 (Gibco-BRL, Rockville, USA) was cultured in lysogeny broth

(LB) medium (Bertani, 1951. J. Bacteriol. 62, 293-300) at 37° C. Tetracycline hydrochloride was used at concentration 10 μg/ml for E. coil and M. extorquens. Cumate (4-isopropylbenzoic acid) was used as inducer with an end concentration of 100 μM diluted from a 100 mM stock solution dissolved in ethanol (culturing in the shaker flask) or methanol, (culturing in the bioreactor).

Cumate, tetracycline hydrochloride, α-humulene, zerumbone and (RS)-mevalonic acid lithium salt were purchased from Sigma-Aldrich (Steinheim, Del.). Dodecane was purchased from VWR (Darmstadt, Del.).

1.2 Genetic Manipulations and Plasmid Construction

The standard cloning techniques were performed according to the procedures known to those skilled in the art. The transformation of plasmids into M. extorquens AM1 or CM502 was performed as described in Toyama et al. (Toyama et al., 1998, FEMS Microbiol. Lett. 166, 1-7).

Ribosome binding sites (RBS) were designed with the aid of the ribosome binding site calculator (Sails, 2011, Methods in Enzymology, ed. V. Christopher, 19-42. Academic Press). The codon adaptation index (CAI) was determined with the CAI calculator (Puigbo et al., 2008, BMC Bioinformatics. 9, 65).

1.3 Cloning of Mevalonate Pathway (MVA) Genes from Myxococcus xanthus

Genomic DNA from Myxococcus xanthus DSM16525 was purchased from DSMZ

(Braunschweig, Del.), The EcoRI restriction site of hmgs, coding for HMG-CoA synthase, was removed by Overlap extension PCR with insertion of a silent mutation (gagttc to gagttc). For this, the first part of the gene was amplified by means of the primers HMGS-fw and HMGS-over-rev, while HMGS-over-fw and HMGS-rev were used for the second part. The resulting PCR products were utilized as “mega” primers together with HMGS-fw and HMGS-rev for the final amplification of hmgs (SEQ ID No. 7) without the EcoRI restriction site.

The mevalonate pathway operon from M. xanthus—containing the genes hmgr (SEQ ID No. 8), mvaK1 (SEQ ID No. 9), mvaK2 (SEQ ID No. 10), mvaD (SEQ ID No. 11) and fni (SEQ ID No. 12) coding respectively for HMG-CoA reductase, mevalonate kinase, phosphomevalonate kinase, pyrophosphomevalonate reductase and isopentenyl-pyrophosphate isomerase, was cleaved out of the plasmid pUC18-mva-op (Mi et al., 2014, Microbial cell factories. 13, 170).

1.4 Cloning of Plasmids Containing the α-Humulene Synthase

The multiple cloning site of plasmid pQ2148 (Kaczmarczyk et al., 2013, Appl. Environ. Microbiol. 79, 6795-802) was modified for increased cloning flexibility. For this, primers pQF-MCS-fw and pQF-MCS-rev were annealed by heating 100 μl annealing buffer (10 mM TRIS pH7.5, 50 mM NaCl, 1 mM EDTA) containing 10 μM of each primer for 15 mins followed by slow cooling to room temperature for three hours. The annealed primers were ligated into pQ2148 which had been cleaved with Spel and Xhol, yielding plasmid pQ2148F.

The α-humulene synthase gene zssl, originally deriving from Zingiber zerumbet (Yu et al., 2008, Planta. 227, 1291-9) (Accession number AB263736.1), was codon-optimized for M. extorquens AM1 with obtention of the DNA sequence according to SEQ ID No. 16. The codon-optimized gene according to SEQ ID No. 16 was amplified for insertion into pCM80 (Marx and Lidstrom, 2001, Microbiology. 147, 2065-2075) and pHC115 (Chou and Marx, 2012, Cell reports. 1, 133-40) using the primers ZSSI-fw and ZSSI-rev. An RBS-optimized variant (translation initiation rate (TIR) of 221,625) for pQ2148F was amplified using the primers ZSSI-RBS-fw and ZSSI-rev. The RBS-optimized variant of zssl with a TIR of 221,625 contains the nucleic acid sequence AGCTTAAGGATAAAGAAGGAGGTAAAAC (SEQ ID No. 41). The gene for the FPP synthase ERG20 from Saccharomyces cerevisiae was amplified from genomic DNA with the primers ERG20-fw and ERG20-rev. RBS-optimized variants were amplified with primers ERG20-RBS35k-fw or ERG20-RBS20k-fw in combination with ERG20-rev-2 resulting in two ERG20 PCR products, each having an RBS with a TIR of 36,800 or 22,000. The RBS-optimized variant of ERG20 with a TIR of 22,000 contains the nucleic acid sequence ACATCAAACCAAAGGACTTTACAGGTAGTAGAA (SEQ ID No. 39). The RBS-optimized variant of ERG20 with a TIR of 36,800 contains the nucleic acid sequence GAGAAGAGCAGACTCGATCATAACAGGGGACTAG (SEQ ID No. 40).

The zssl PCR product was digested with Sphl and Xbal and inserted into identically digested plasmid pCM80, yielding plasmid pFS33. C/al and Snaal digested PCR product from ERG20 was then cloned into the same restriction sites of pFS33 resulting in pFS34. The hmgs gene without the EcoRl restriction site (see above) was inserted behind ERG20 using the restriction cleavage sites Xbal and BamH1. The M. xanthus mevalonate operon was cleaved out of pUC18-mva-op with BamH1 and EcoRl and reinserted into identically digested pFS34-hmgs yielding pFS44.

The plasmids pFS45 (pHC115-zssl), pFS46 (pHC115-zssl-ERG20) and pFS47 (pHC115-zssl-ERG20-hmgs-MVAop) were constructed by cleaving zssl out from pFS33, zssl-ERG20 out from pFS34 and zssl-ERG20-hmgs-MVAop out from pFS44 with MH and EcoRl followed by their insertion into identically digested pHC115.

The plasmids pFS49 (pQ2148F-zssl) and pFS50 (pQ2148F-zssl-ERG20) were constructed by cleaving zssl and zssl-ERG20 out from pFS33 and pFS34 respectively with Af/ll and Xbal followed by their insertion into identically digested pQ2148F. Hmgs and MVAop were cleaved out from pFS44 by Xba1 and EcoRl and subsequent insertion into the same restriction sites of pFS50 resulted in pFS52 (pQ2148F-zssl-ERG20-hmgs-MVAop).

The PCR product of the α-humulene synthase gene zssl with optimized RBS was digested with Spel and Xbal and ligated into identically digested pQ2148F yielding pFS57. The PCR product of ERG20 with optimized RBS was cloned behind zssl from pFS57 with Clal and Xbal resulting in pFS58 (pQ2148F-zssl^{RBSopt}_-ERG20). Hmgs-MVAop was inserted into pFS58 as described for pFS52 yielding pFS59. RBS variants for ERG20 (TIR=35,000 and 20,000) were digested with Clal and Xbal and inserted into correspondingly cleaved pFS57 yielding pFS60a (zssl^{RBSopt}_-ERG20^{35k}) and pFS60b (zssl^{RBSopt}_-ERG20^{20k}) respectively. Insertion of hmgs-MVAop into pFS60a and pFS60b resulted in plasmids pFS61a (zssl^{RBSopt}_-ERG20^{35K}-hmgs-MVAop) and pFS61b (zssl^{RBSopt}_-ERG20^{35k}-hmgs-MVAop) respectively. The RBS of the IPP isomerase gene fni was optimized for pFS61a and pFS61 b by insertion of initially annealed primers fni-RBSopt-fw and fni-RBSopt-rev (annealing method see above) into restriction sites Hpal and BamH1. The resulting plasmids pFS62a and pFS62b have a TIR of 65,000 for the fni RBS. The optimized RBS for the gene fni here has the nucleotide sequence gttctaggaggaataata (SEQ ID No. 48). The optimized RBS for the gene hmgs in plasmids pFS61a and pFS61b and also pFS62a and pFS62b has the nucleotide sequence SEQ ID No. 90 with a TIR of 189.

An overview of the primers, plasmids and strains used is shown in Table 1,

TABLE 1
Primers, plasmids and strains used.

| | | |
|---|---|---|
| Name | Description | Reference |

Primers

| | |
|---|---|
| HMGS-fw | AGTCTAGAGAGGAGCGCAGGATGAAGAAGCGCGTGGGAAT |
| | (SEQ ID No. 17) |
| HMGS-rev | ATCTGGATCCGTTTAAACCCTGCAGGACCGGTGTTAACTCAG |
| | TTCCCTTCGGCGTAC (SEQ ID No. 18) |
| HMGS- | GCTGCGCGGCCGAGTTCTACTCCGGCACG (SEQ ID No. 19) |
| over-fw | |
| HMGS- | CGTGCCGGAGTAGAACTCGGCCGCGCAGC (SEQ ID No. 20) |
| over-rev | |
| MVA1_fw | ATCTGGATCCTAGGAGGAATAATATGGGCGACGACATCACT |
| | G (SEQ ID No. 21) |
| MVA- | AACACCATGGCGAGCTCTC (SEQ ID No. 22) |
| SacIA-rev | |
| MVA- | GAGAGCTCGCCATGGTGTT (SEQ ID No. 23) |
| SacIA-fw | |
| MVA- | GTGCCCGTTGAGCTCCACCT (SEQ ID No. 24) |
| SacIB-rev | |
| MVA- | AGGTGGAGCTCAACGGGCAC (SEQ ID No. 25) |
| SacIB_fw | |
| MVA2_rev | ATCGAATTCAAGCTTTCAGCTCAGCGCGCGCACC (SEQ ID |
| | No. 26) |
| pQF_MCS- | CTAGTCTGCAGCTTAAGCATGCTCTAGAAGATC (SEQ ID No. |
| fw | 27) |
| pQF_MCS- | TCGAGATCTTCTAGAGCATGCTTAAGCTGCAGA (SEQ ID No. |
| rev | 28) |
| ZSSI-fw | TAGCATGCTTAAGAAGGATCAGTCATAATGGAACGCCAGTC |
| | GATGG (SEQ ID No. 29) |
| ZSSI-RBS- | ATACACTAGTAGCTTAAGGATAAAGAAGGAGGTAAAACATG |
| fw | GAACGCCAGTCGATGG (SEQ ID No. 30) |
| ZSSI-rev | AGTCTAGATACGTAATCGATTCAGATGAGGAACGACTCGA |
| | (SEQ ID No. 31) |
| ERG20_fw | ATCGTATCGATAGGAGCGCAGGATGGCTTCAGAAAAAGAAA |
| | TTAG (SEQ ID No. 32) |
| ERG20-RBS | ATCGTATCGATGAGAAGAGCAGACTCGATCATAACAGGGG |
| (35k)-fw | ACTAGATGGCTTCAGAAAAAGAAATTAG (SEQ ID No. 33) |
| ERG20-RB | ATCGTATCGATACATCAAACCAAAGGACTTTACAGGTAGTA |
| S(20k)-fw | GAAATGGCTTCAGAAAAAGAAATTAG (SEQ ID No. 34) |
| ERG20_rev | atcgtacgtaCTATTTGCTTCTCTTGTAAACT (SEQ ID No. 35) |
| ERG20_rev- | ACTATCTAGATAAAGTAGAGGAGGATTAATCTATTTGCTTCTC |
| 2 | TTGTAAACT (SEQ ID No. 36) |
| fni-RBSopt- | AACCTAAAATTAACGAGGAAAGAGGGAGGTTACAG (SEQ ID |
| fw | No. 37) |
| fni-RBSopt- | GATCTGTAACCTCCCTCTTTCCTCGTTAATTTTAGGTT (SEQ |
| rev | ID No. 38) |

Plasmids

| | | |
|---|---|---|
| pUC18 | Expression vector for Escherichia coli; Amp^{R}, lacZ promoter, | Norrander |
| | pBR322ori | 1983 |
| pACCRT- | Plasmid, for expression of diapophytoene synthase and | Sandmann |
| MN | desaturase in Escherichia coli; Amp^{R}; contains genes for | |
| | diapophytoene synthase (crtM) and diapophytoene desaturase | |
| | (crtN) from Staphylococcus aureus under control of a lacZ | |
| | promoter | |
| pCM80 | Constitutive expression vector for Methylobacterium extorquens; | Marx |
| | Tet^{R}, pmxaF, oriT, pBR322ori | 2001, |
| | | Microbiology. |
| | | 147, |
| | | 2065- |
| | | 2075. |
| pHC115 | Expression vector for Methylobacterium extorquens with cumate | Chou |
| | inducible pmxaF promoter variant; Kan^{R}, oriT, pBR322ori | 2012, Cell |
| | | reports. 1, |
| | | 133-40. |
| pQ2148 | Expression vector for Methylobacterium extorquens with cumate | Kaczmarczyk |
| | inducible promoter 2148; Tet^{R}, oriT, pBR322ori | 2013, |
| | | Appl. |
| | | Environ. |
| | | Microbial. |
| | | 79, 6795- |
| | | 802. |
| pQ2148F | pQ2148 with adapted MCS | |
| pUC18- | pUC18 with Myxococcus xanthus MVA operon (hmgr, mvaK, | |
| MVAop | mvaK2, mvaD, fni) | |
| pCM80- | pCM80 with hydroxymethylglutaryl synthase hmgs | |
| HMGS | | |
| pCM80- | pCM80 with complete mevalonate (MVA) pathway | |
| MVA | | |
| pCM80- | pCM80 with complete mevalonate pathway and FPP synthase | |
| MVA- | ERG20 | |
| ERG20 | | |
| pFS33 | pCM80 with alpha-humulene synthase zssl | |
| pFS34 | pCM80 with alpha-humulene synthase zssl and FPP synthase | |
| | ERG20 | |
| pFS44 | pFS34-hmgs-MVAop | |
| pFS45 | pHC115 with alpha-humulene synthase zssl | |
| pFS46 | pHC115 with alpha-humulene synthase zssl and FPP synthase | |
| | ERG20 | |
| pFS47 | pFS46-hmgs-MVApp | |
| pFS49 | pQ2148F with alpha-humulene synthase zssl | |
| pFS50 | pQ2148F with alpha-humulene synthase zssl and FPP synthase | |
| ERG20 | | |
| pFS52 | pFS50- hmgs-MVAop | |
| pFS57 | pQ2148F with alpha-humulene synthase zssl with optimized RBS | |
| pFS58 | pFS57-ERG20 | |
| pFS59 | pFS58- hmgs-MVApp | |
| pFS60a | pFS57-ERG20^{35k} (RBS with au of 35,000) | |
| pFS60b | pFS57-ERG20^{20k} (RBS with au of 35,000) | |
| pFS61a | pFS60a-hmgs-MVAop | |
| pFS61b | pFS60b-hmgs-MVAop | |
| pFS62a | pFS61a with optimized RBS of the IPP isomerase fni | |
| pFS62b | pFS61b with optimized RBS of the IPP isomerase fni | |

Strains

| | | |
|---|---|---|
| E. coli | F-, ϕ80dlacZΔM15, Δ(lacZYA-argF)U169, deoR, recA1, endA1, | ATCC |
| DH5α | hsdR17(rK⁻mK⁺), phoA, supE44, λ⁻, thi-1 | |
| M. | Facultatively methylotrophic, obligatorily aerobic, gram-negative, | Peel & |
| extorquens | pink pigmented a-proteobacterium, Cm^{R} | Quayle |
| AM1 | | 1961, |
| | | Biochem |
| | | J, 81, 465- |
| | | 9. |
| | | DSM1338 |
| M. | Carotenoid biosynthesis deficient strain | Van Dien |
| extorquens | | et al., |
| CM502 | | 2003, |
| | | Appl. |
| | | Environ. |
| | | Microbiol. |
| | | 69, 7563- |
| | | 6. |
| Saccharomyces | MATa; ura3-52; trp1-289; leu2-3,112; his3Δ 1; MAL2-8^{c}; SUC2 | Entian & |
| cerevisiae | | Mittel- |
| CEN.PK2- | | 1998, |
| 1c | | Academic |
| | | Press Ltd., |
| | | San |
| | | Diego, pp. |
| | | 431-449 |

Underlined and italic sequences (all 5′ → 3′) indicate recognition sites for restriction enzymes. Bold letters show sequences of ribosome binding sites (RBS), au: translation initiation rate (TIR) according to Salis Lab RBS calculator; op: operon, MVA: mevalonate pathway.

1.5 α-Humulene Production in Aqueous Organic Two Phase Shaker Flask Culture Methylobacterium extorquens AM1 or CM502, containing the α-humulene production plasmids, were cultured in methanol minimal medium containing tetracycline hydrochloride (see above). Precultures were inoculated from agar plates into test tubes with 5 ml medium and shaken for 48-72 h at 30° C. and 180 rpm. Main cultures with 12 ml medium in 100 ml baffled shaker flasks were inoculated with a preculture to an OD600 of 0.1. After culturing for 16 h at 30° C. and 120 rpm the main cultures reached the early exponential growth phase (OD₆₀₀0.3-0.6). Next, cumate was added for the induction and 3 ml dodecane added as organic phase. After 48 h incubation a total culture volume of 15 ml was decanted and centrifuged for 10 min at 3220 g. 1 ml of the upper dodecane layer was used for the α-humulene analysis. The cell pellet was resuspended in 1 ml dH₂O for intracellular α-humulene analysis.

1.6 Dodecane and α-Humulene Tolerance of M. extorquens AM1

M. extorquens AM1 precultures were cultured in test tubes with 5 ml methanol minimal medium (MM) for 48 h. The tolerance of M. extorquens AM1 towards 20% (v/v) dodecane was studied by growth comparison ( OD₆₀₀) of cultures containing 15 ml MM and cultures with 12 ml MM and 3 ml dodecane. The cultures for the growth comparison with and without dodecane were inoculated from one preculture.

The α-humulene tolerance was tested in two ways: tolerance towards α-humulene added directly to the aqueous phase and tolerance towards α-humulene dissolved in the organic dodecane layer. For the first experiment, pure α-humulene dissolved in ethanol was added to 100 ml baffled shaker flasks containing 15 ml MM with end concentrations of α-humulene of 1000, 500, 250, 100, 50, 25, 10 and 5 mg/L. Corresponding quantities of ethanol were added to the MM as negative controls. The flasks with the different α-humulene concentrations and the corresponding negative controls were inoculated with a preculture without α-humulene to an OD₆₀₀ of 0.1. The OD₆₀₀ was recorded over 30 h.

For the second said experiment, pure α-humulene was dissolved in dodecane and solutions with 1000, 500, 100, 50 and 10 mg/L α-humulene prepared. Two cultures each with 12 ml MM and 3 ml dodecane for each α-humulene concentration were inoculated to an OD₆₀₀ of 0.1 from a preculture of M. extorquens AM1 without dodecane. Cultures with dodecane without α-humulene were used as negative controls. OD₆₀₀ was measured over 30 h.

1.7 α-Humulene Analysis

1 ml dodecane sample was dried with NaSO₄. As the internal standard 25 μl of 1 mM zerumbone dissolved in dodecane were added to 225 μl dodecane sample.

Intracellular α-humulene was extracted as follows: resuspended cell pellet was placed in a 4 ml GC vessel together with ca. 300 mg of 0.2 mm glass balls. The cells were intensively vortexed 3×30 s with interim ice cooling. The lysed cells were extracted three times with 1 ml hexane followed by a volume reduction to 1 ml by means of a current of nitrogen. As the internal standard, 25 μl of 1 mM zerumbone dissolved in hexane was added to 225 μl sample.

α-humulene was analyzed and quantified by means of GC-MS (GC17A with Q5050 Mass Spectrometer, Shimadzu, Kyoto, Japan) equipped with an Equity 5 column (Supelco, 30 m×0.25 mm×0.25 μM). Measurements were performed twice as follows: carrier gas: helium; split injection (8:1) at 250° C.; flow rate: 2.2 ml/min; interface temperature: 250° C.; program: 80° C. hold for 3 mins, 16° C./min to 240° C., hold for 2 mins. The retention time was 9.3 mins for α-humulene and 11.5 min for zerumbone, α-Humulene in the samples was identified by comparison of three main fragmentations of the mass spectra with a commercially obtained α-humulene standard (rel. intensity in brackets): 93 (15.5), 41 (11.4), 80 (6.7). For a quantification, a calibration curve with the concentrations 4500, 2250, 900, 675, 450, 225, 90, 67.5, 22.5, 9 and 4.5 μM α-humulene each with 100 μM zerumbone was used.

1.8 Carotenoid Extraction and Analysis

For the carotenoid extraction, cells of M. extorquens AM1 or E. coli were pelleted by centrifugation, washed with ddH₂O and lyophilized in the dark.

For unsaponified extracts, 2 mi methanol were added to 50 mg of disintegrated freeze-dried cells with subsequent incubation at 65° C. for 30 mins. After centrifugation (10 mins, 4000g, 4° C.) the supernatant was dried with nitrogen and resuspended in 0.5 ml of a petroleum ether (40-60° C.): diethyl ether: acetone: methanol (40:10:15:5) mixture. Precipitated proteins were removed by centrifugation (5 mins, 16,000g, 4° C.) and the supernatant was taken up in 100 μl tetrahydrofuran (THF) for the HPLC analysis after drying with nitrogen. For saponified extracts, after protein removal with 10% KOH solution (dissolved in methanol) the supernatant was incubated for 2 h at RT. The upper organic phase was then dried with nitrogen and taken up in 100 μl THE for the HPLC analysis.

The HPLC analysis was performed with a Shimadzu SCL10 system (SPD10A UVNIS detector, SPD-M10A diode array detector, SIL10A autosampler, CTO-10AC column oven; each Shimadzu, Kyoto, Japan). Carotenoids were separated on a reverse phase C18 column (250 mm×4.5 mm×5 μ; Alltech, Deerfield, USA) using a gradient program of acetonitrile: methanol: 2-propanol (85:10:5) as solvent A and 100% 2-propanol as solvent (Solv.) B. At a flow rate of 1 ml/min at 32° C. the following elution program was run: 100% Solv. A, 0% Solv. B 0-31 min, 0% Solv. A, 100% Solv. B 31-36 min, 100% Solv. A and 0% Solv. B 36-45 min. A wavelength range of 190-600 nm was monitored by diode array detector. The retention time of lycopene was 25.38 mins. Diapolycopene was identified via a comparison with a carotenoid extract from E. coli, which expresses diapophytoene synthase and diapophytoene desaturase from Staphylococcus aureus via pACCRT-MN (see Table 1).

1.9 Fermentation

Fed batch cultures were performed in a 2.4 I KLF 2000 fermenter (Bioengineering AG, Wald, Switzerland) with a pH and pO₂ electrode from Mettler-Toledo (Greifensee, Switzerland), two six-paddle turbine stirrers and a downward directed paddle stirrer. Filter-sterilized air or oxygen was provided at a flow rate of 50 l/h. All experiments were performed at 30° C. and at a pH of 6.75, which was regulated by automatic introduction of NH₄OH (30%). The concentration of dissolved oxygen (DO) was automatically regulated by adjustment of the stirring speed beginning at 700 rpm. Oxygen and carbon dioxide were measured in the exhaust air with a BINOS 1001 gas analyzer (Rosemount Analytical, Hanau, Del.).The methanol concentration was monitored online and regulated half-hourly with a ProcessTRACE 1.21 MT system (Trace Analytics, Braunschweig, Del.), equipped with a dialysis probe. The methanol feed was configured as follows: below a concentration of 1 g/I, 0.79 g (1 ml) and below 0.5 g/I, 1.42 g (1.8 ml) methanol was introduced via a Watson-Marlow 505Du peristaltic pump (Cornwall, England). Anti-foam B emulsion (Sigma-Aldrich) was manually added to reduce foaming, in addition to a six-paddle turbine stirrer, which is mounted directly over the liquid phase as a mechanical foam breaker.

After in situ sterilization of 900 ml fermentation medium (see above), the fermenter with an OD₆₀₀ of 0.5-1 was inoculated with a preculture which has grown for 72 h in a shaker flask. After attainment of an OD₆₀₀ of 5-10, 100 μM cumate from a freshly prepared stock solution in methanol and 15% dodecane were added. The methanol feed rate was doubled after the induction. The induced culture was further cultured for 120 h, and samples were withdrawn manually, the cell dry mass and OD₆₀₀ thereof were determined from the aqueous phase and α-humulene in the organic dodecane phase were measured as described above.

2. Results

2.1 α-Humulene Production Using Plasmids with Constitutive Promoter (Comparative Example)

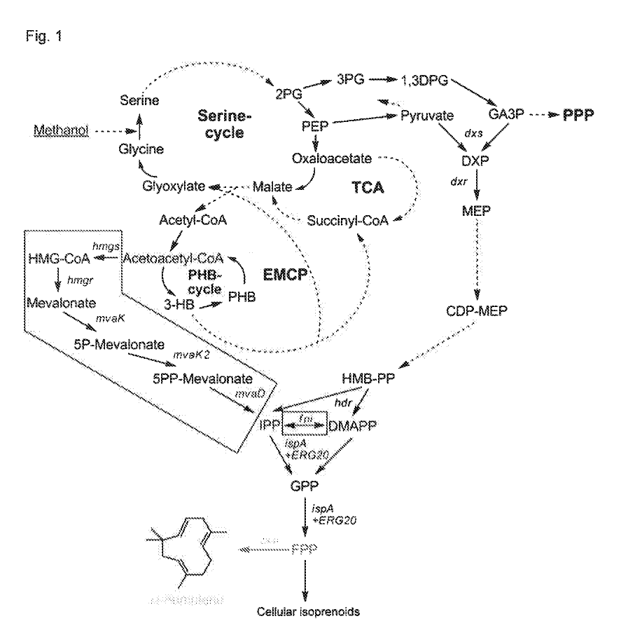

Methylobacterium extorquens AM1 endogenously produces a farnesyl pyrophosphate (FPP) pool which is however converted into menaquinone, hopanes and carotenoids (see FIG. 1). In principle, this bacterium could synthesize α-humulene through integration of a heterologous α-humulene synthase. Plasmid pCM80 bears the strong pmxaF promoter and was selected as the vector for the expression of the α-humulene synthase gene zssl. A codon-optimized variant of the gene from Zingiber zerumbet was introduced into in pCM80 with obtention of pFS33. To further increase the α-humulene production, the FPP synthase from Saccharomyces cerevisiae (ERG20) was cloned into pFS33 behind the zssl gene with obtention of pFS34 (pCM80-zssl-ERG20).

The culturing was performed under the conditions described above, as aqueous organic two phase cultures, wherein dodecane is used as the organic phase. The strong hydrophobicity of α-humulene results in complete accumulation in the dodecane phase, since intracellular α-humulene was not detectable.

Two advantages in particular derive from this: α-humulene concentrations can be measured directly in the dodecane phase and evaporation of α-humulene is decreased by the high boiling point of dodecane. Furthermore, M. extorquens AM1 tolerates 20% dodecane, without any toxic effects or influence on growth being observable.

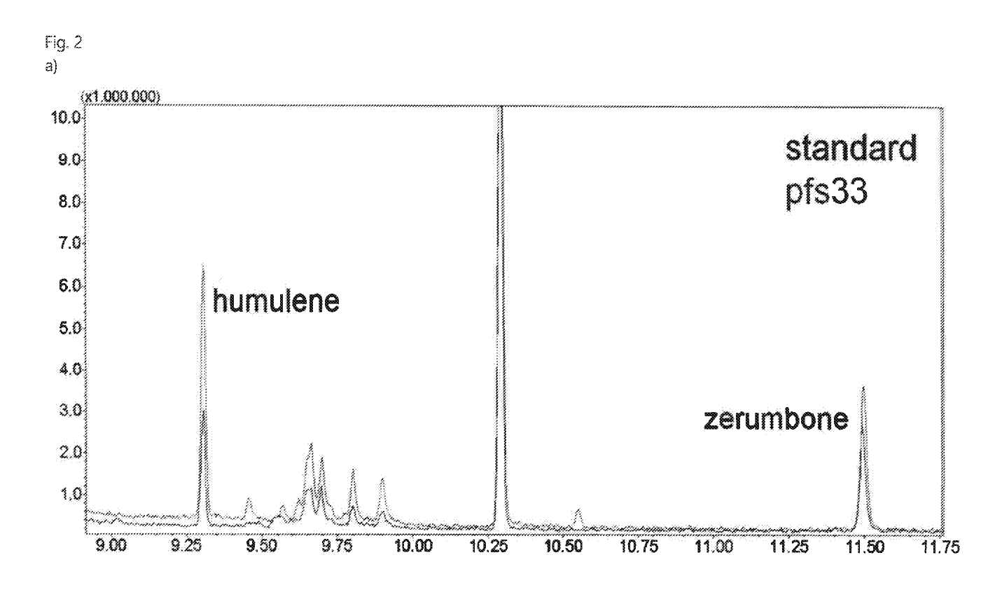

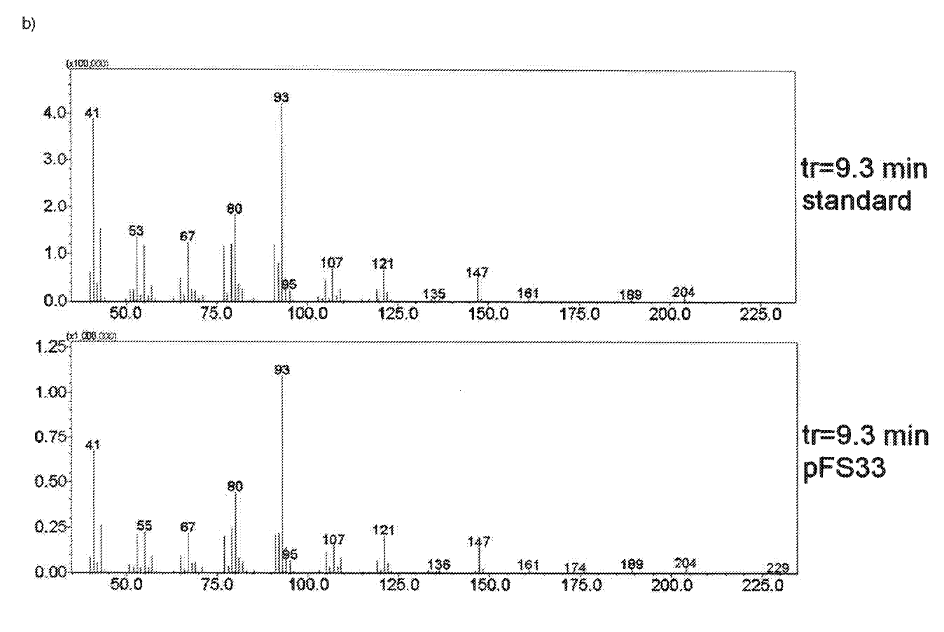

M. extorquens AM1 (also abbreviated below as: AM1) containing plasmid pFS33 was able to produce α-humulene, as is shown by the peak with similar retention time and mass spectrum in comparison to the α-humulene standard in FIG. 2. In contrast to this, no α-humulene is detectable for M. extorquens AM1 with the empty vector control.

Both for AM1_pFS33 and also AM1_pFS34, 2.3 mg/L α-humulene were measured. The FPP synthase ERG20 from pFS34 appears not to increase the α-humulene concentration. The more detailed reasons for this are not known.

2.2 Integration of the Heterologous Mevalonate Pathway (MVA)

Surprisingly, however, here it could be found that the heterologous expression in particular of enzymes of the MVA pathway leads to improved formation of α-humulene.

This is all the more astonishing since the MVA precursor, acetoacetyl-CoA, is a component of the primary metabolism of M. extorquens (see FIG. 1). Through the withdrawal of acetoacetyl-CoA for the heterologously introduced MVA pathway, a considerable imbalance in the primary metabolism of M. extorquens was to be expected.

This fear was at first substantiated by the following preliminary experiment. The transformation of pFS44 containing zssl, ERG20 and the M. xanthus MVA genes hmgs, fni, hmgr, mvaK, mvaK2 and mvaD into electrocompetent M. extorquens AM1 yielded no discernible growth, neither on methanol minimal medium nor on succinate minimal medium. The constitutive expression of the MVA pathway appears not to be well tolerable for M. extorquens.

2.3 Toxicity of α-Humulene to M. extorquens AM1

In order to establish whether M. extorquens is suitable at all as a production strain for terpenes, it was firstly checked whether the bacterium is inhibited in growth by terpenes in higher concentrations.

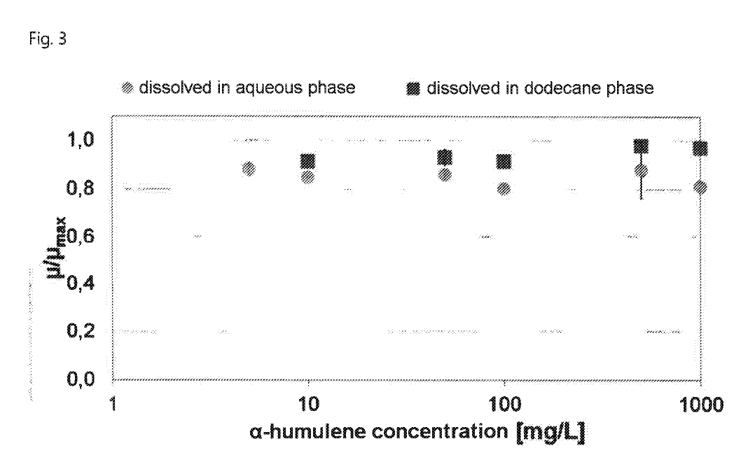

Terpenes often have toxic effects on bacteria. The toxicity of α-humulene to M. extorquens was studied by growth analyses in the presence of different α-humulene concentrations. A dodecane layer containing α-humulene was added to M. extorquens cultures as a second phase. In a second approach, α-humulene was added directly to the aqueous phase. The results presented in FIG. 3 show that M. extorquens is suitable as a production platform for terpenes, in particular for α-humulene.

2.4 α-Humulene Production Using a Cumate-Inducible Promoter

For the inducible expression of the MVA genes, a suitable plasmid system with inducible promoter was used below. For this, the genes for the α-humulene synthase were cloned alone, in combination with FPP synthase ERG20 and in combination with ERG20 and the MVA pathway genes, into plasmid pHC115, which bears a cumate inducible promoter (Chou and Marx, 2012), yielding the plasmids pFS45, pFS46 and pFS47 respectively.

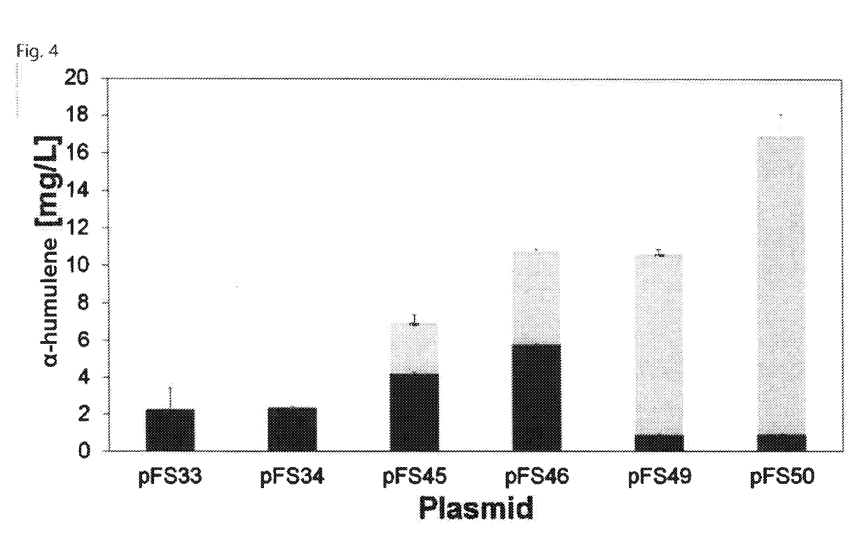

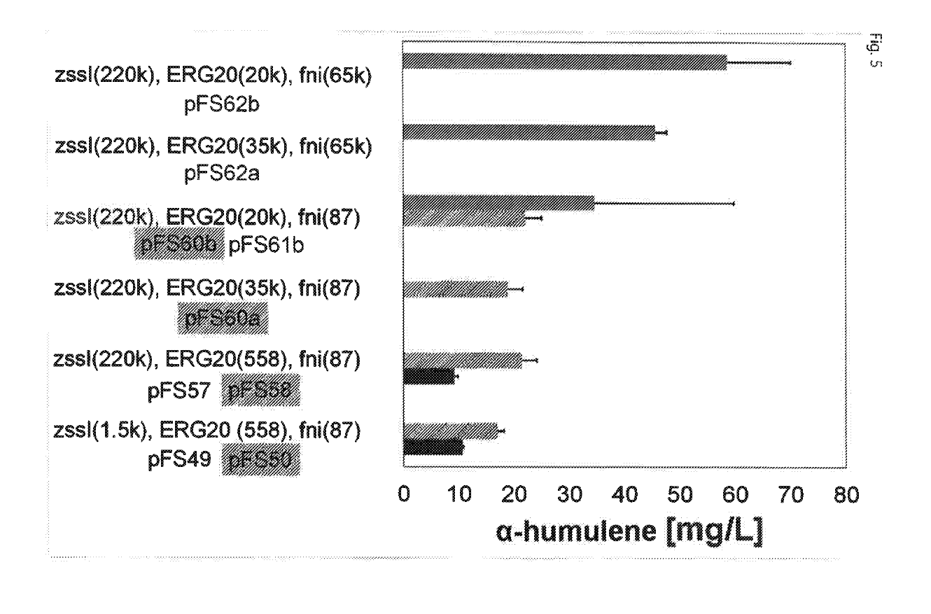

After transformation, colonies were obtained for pFS45 and pFS46, but scarcely detectable for pFS47. Without being bound thereto, the data shown in FIG. 4 for pFS45 and pFS46 could explain this: more than 50% α-humulene was already produced without induction. The gene expression of pHC115 is not tight and remaining expression of the MVA genes has an adverse effect on growth for M. extorquens.

Plasmid pQ2148 contains the very tight cumate inducible 2148 promoter. Zssl alone and once again in combination with ERG20 and with the MVA genes were introduced into pQ2148F with obtention of pFS49 (zssl), pFS50 (zssl-ERG20) and pFS52 (zssl-ERG20-MVA). Colonies were obtained after transformation into M. extorquens for pFS49, pFS50 and also pFS52, even though the colonies for pFS52 were very small even after 8 days growth at 30° C. The α-humulene concentrations reached 11 mg/L in AM1_pFS49 and 17 mg/L in AM1_pFS50 (see FIG. 4), which is a 6-fold or 1.6 times increase in the zssl-ERG20 construct compared to pFS34 and pFS46. In the comparison to pHC115 constructs, the background production, i.e. without induction, was only 5%.

The compensation of flux imbalances in the metabolism can be achieved by a great variety of measures. Thus for example the promoter strength, the concentration of the inducer, the plasmid copy number or combinations thereof can be decisive. Here it was now surprisingly found that the translation initiation rates (TIR) of different ribosome binding sites (RBS) are of importance for an improved terpene synthesis.

Firstly, the TIR of the α-humulene synthase RBS in the plasmids pFS57 (zssl^{225k}), pFS58 (zssl^{225k}-ERG20) and pFS59 (zssl^{225k}-ERG20-MVA) were increased 146-fold (see Table 2).

TABLE 2
Translation initiation rates (TIR) of the native and
optimized ribosome binding sites (RBS) of the heterologous
mevalonate pathway genes hmgs (hydroxymethylglutaryl-CoA
synthase) and fni (IPP isomerase) from Myxococcus xanthus,
the FPP synthase ERG20 and α-humulene synthase zssl of
the various plasmids.

| | Translation initiation rates (TIR) | | | | | |
|---|---|---|---|---|---|---|
| | | | [symbol] fni | | | |
| Plasmids | hmgs | | IPP | | | Growth |
| Gene: | AAc- | HMG- | [symbol] | ERG20 | zssl | α-hu- |
| Intermediate: | CoA → | CoA | DMAPP | → | FPP → | mulene |

| | | | | | |
|---|---|---|---|---|---|
| pFS49 | — | — | wt | 1514 | +++ |
| pFS50 | — | — | 558 | 1514 | ++ |
| pFS52 | 1995 | 87.3 | 558 | 1514 | −/+^{§} |
| pFS57 | — | — | wt | 221625 | ++ |
| pFS58 | — | — | 558 | 221625 | +++ |
| pFS59 | 1995 | 87.3 | 558 | 221625 | −/+^{§} |
| pFS60a | — | — | 36800 | 221625 | ++ |
| pFS60b | — | — | 22000 | 221625 | +++ |
| pFS61b | 6345 | 87.3 | 22000 | 221625 | + |
| pFS62a | 6345 | 65000 | 36800 | 221625 | + |
| pFS62b | 6345 | 65000 | 22000 | 221625 | ++ |

^{§}various colony sizes,
Growth: colony formation of AM1 on methanol agar after transformation: ++++: like empty vector (3-4 days), +++: 4-5 days, ++: 5-6 days, +: 6-7 days, −: no colonies discernible after 8 days;
wt: native FPP synthase from M. extorquens AM1 with unknown RBS;
intermediates: AAc-CoA: acetoacetyl-CoA, HMG-CoA: hydroxymethylglutaryl-CoA, IPP: isopentenyl pyrophosphate, DMAPP: dimethylallyl pyrophosphate, FPP: farnesyl pyrophosphate:

As can be seen in FIG. 5, an optimization of the RBS of zssl alone does not lead to increased α-humulene production without additional provision with precursors of the MVA pathway (pFS57 and pFS58). Transformants with pFS59 (zssl^{225k}-ERG20-MVA) grew slowly, comparably to pFS52-containing strains without zssl RBS optimization (see Table 2).

The TIR of the ERG20 RBS was increased in the ratio of about 1:10 (pFS61b) to the TIR of the zssl RBS (see Table 2). The RBS optimization of ERG20 in combination with zssl RBS optimization did not lead to the increase in the α-humulene formation without MVA (pFS60a and pFS60b, see FIG. 5).

The combination of RBS optimized α-humulene synthase, RBS optimized FPP synthase and MVA enzymes present led to the plasmid pFS61b, which enables good growth (TIR of ERG20 is 22,000). Concentrations of up to 60 mg/L α-humulene were reached by some AM1 transformants with pFS61b (average production was 35 mg/L), even though high fluctuations were to be observed.

Optimizations of the RBS of the IPP Isomerase led to a further increase in the average α-humulene production and to a diminution of the high fluctuations in the α-humulene production between the transformants. The TIR of the fni RBS was increased in plasmids pFS62a and pFS62b to 65,000 (see Table 2). The strains AM1_pFS62b and AM1_pFS62a show further improved growth compared to AM1_pFS61b.

With strain AM1_pFS62b, concentrations of 58 mg/L α-humulene were formed with significantly reduced variance between the transformants in comparison to AM1_pFS61b (see FIG. 5). The optical density was about 3 after 48 h induction, which corresponds to a cell dry weight of 1 g/I.

The heterologous expression of the MVA pathway in M. extorquens was effected according to the last described embodiments by adaptation of the RBS of the α-humulene synthase, the FPP synthase and the IPP isomerase. Concentrations of 58 mg/L α-humulene were reached by M. extorquens containing pFS62b (zssl^{220k}-ERG20^{20k}-fnl^{65k}-MVA). This is at any rate a threefold increase compared to a strain with overexpressed α-humulene synthase and overexpressed FPP synthase in the absence of the heterologous MVA pathway.

2.5 α-Humulene Production in Carotenoid Biosynthesis Deficient M. extorquens Strains

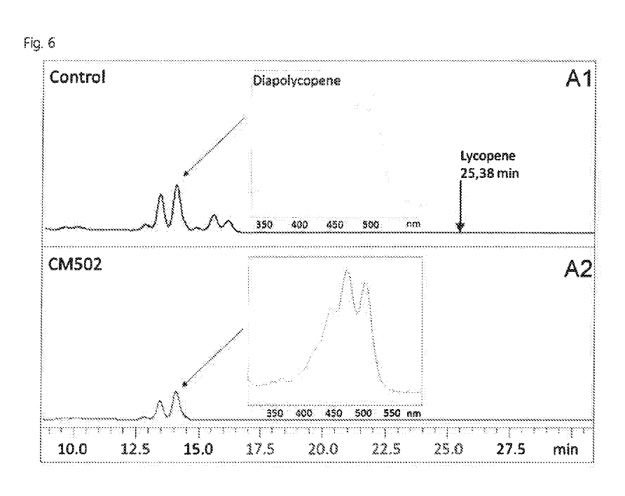

The carotenoid biosynthesis in M. extorquens competes with the α-humulene synthase for the precursor FPP (see FIG. 1). The use of a carotenoid synthesis deficient mutant might be able according to a further practical example to increase the α-humulene production further. For this, the colorless M. extorquens AM1 mutant strain CM502 (Van Dien et al. 2003) was. The carotenoid extraction and analysis (see above) from strain CM502 showed that it produces diapolycopene, but no lycopene, which has an identical UV spectrum, but a different retention time (see FIG. 6A). The data indicate that the strain CM502 is a diapolycopene oxidase mutant (crtNb), since it still produces diapolycopene, but no esterified/glycosylated derivatives.

The α-humulene production of strain ΔcrtNb with plasmid pFS62b was once more significantly increased by about 30% to M. extorquens AM1 wild type with plasmid pFS62b (see FIG. 6B). A production titer of at any rate 75 mg/I α-humulene in the shaker flask could thus be achieved.

It is noteworthy here that the aforesaid concentrations, such as for example 58 mg/L or 75 mg/L α-humulene, are already reached without for example costly lithium acetoacetate or DL-mevalonate having to be added externally. It is moreover advantagous that the aforesaid concentrations were already achieved with use of inexpensive methanol minimal medium. In contrast to the prior art, no TB or LB-based fermentation medium is necessary. This results in a further advantage in the simplification of the purification of the terpene products obtained, since a clearly defined minimal medium can be used. Laborious removal of side products can be minimized. In addition, the strains described here open up the use of *Methanol as the sole carbon source for growth.

2.6 α-Humulene Production in Fed Batch Cultures

In order to test the productivity of the M. extorquens-based α-humulene production according to the invention, methanol-limited fed batch fermentations were performed. The aqueous organic two phase cultures described above were utilized.

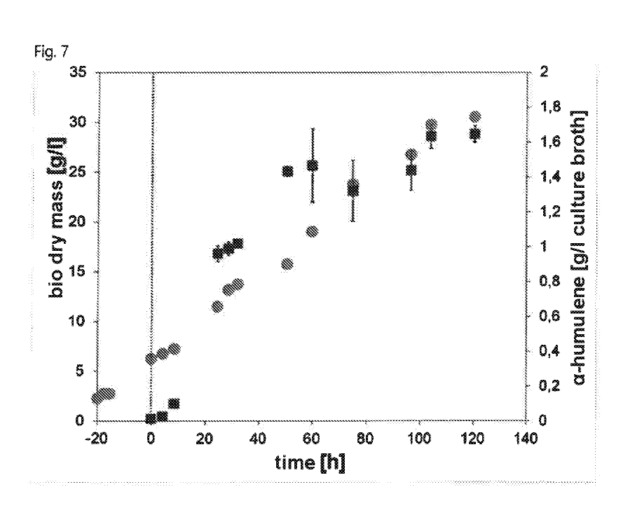

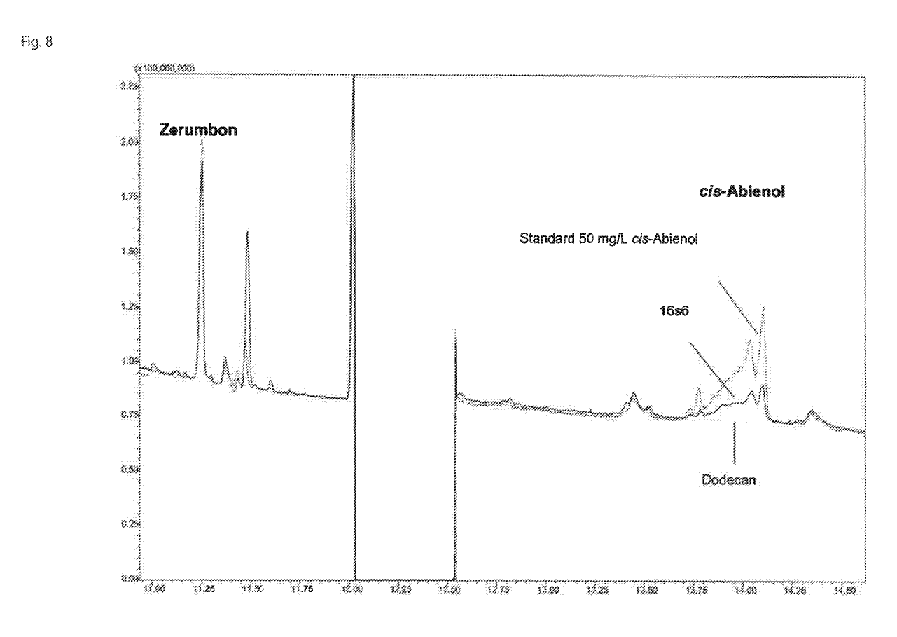

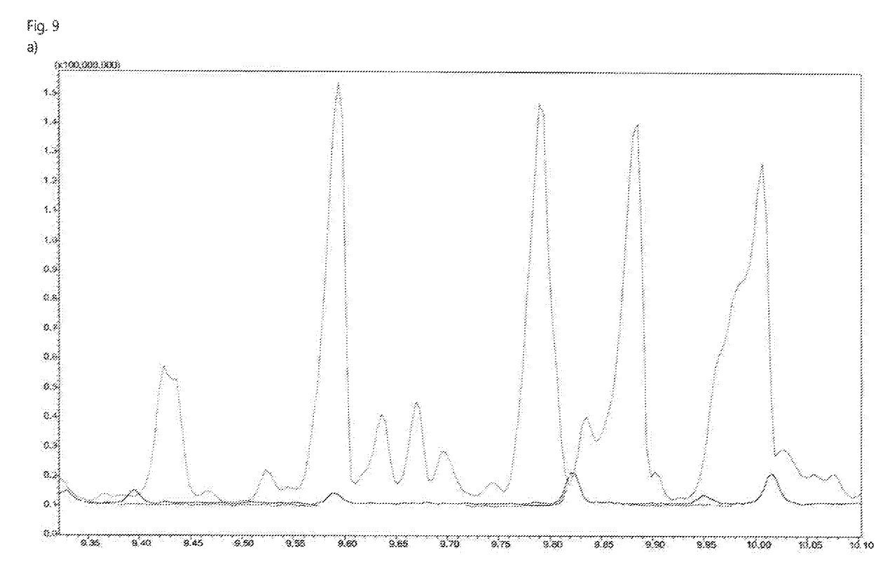

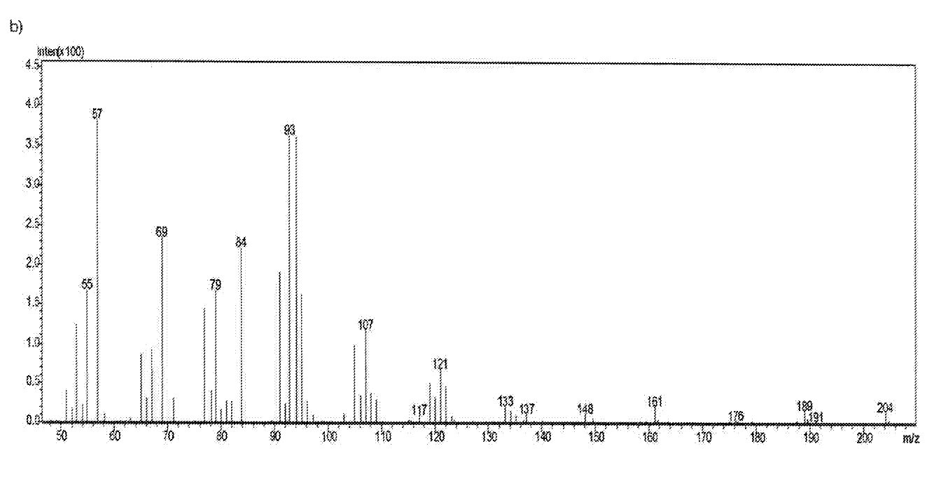

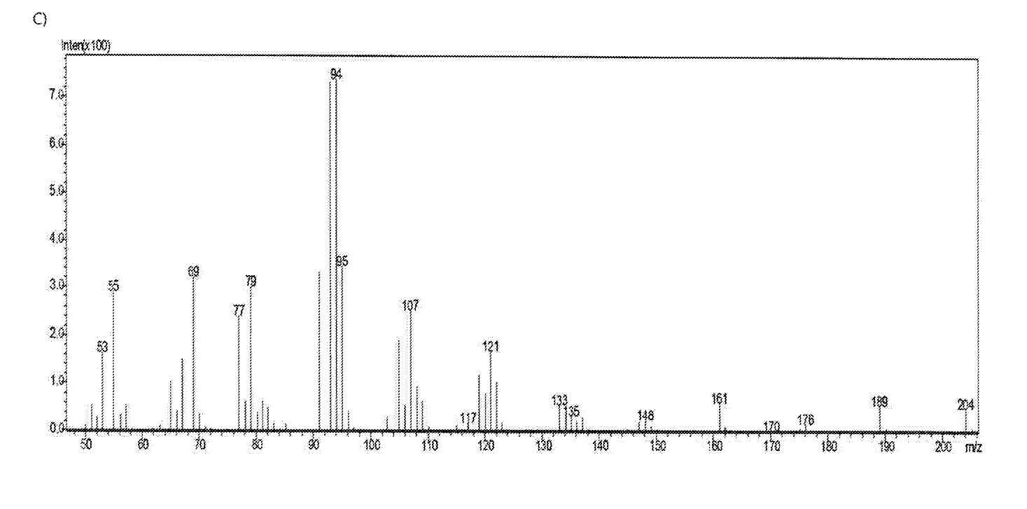

M. extorquens AM1 or ΔcrtNb containing the plasmid pFS62b were grown up to an OD600 of 5-10 before expression of the α-humulene synthesis pathway was induced with cumate and a dodecane phase was added. The further culturing took place at constant pH, dissolved oxygen level of >30% and methanol concentrations of about 1 g/L. Average OD60 values of 80-90 were achieved per fermentation (see Table 3) corresponding to a cell density of about 30 g/I. As shown in FIG. 7, the α-humulene production was growth-dependent. High α-humulene concentrations of 0.73 g/I to 1.02 WI were formed by strain M. extorquens AM1 with plasmid pFS62b. A maximum α-humulene concentration of 1.65 g/I was formed by strain M. extorquens ΔcrtNb with plasmid pFS62b, a 57% increase compared with the highest concentration of 1.02 WI by strain AM1 with plasmid pFS62b (see Table 3). The maximum product concentration of 1.65 g/I signifies a 22 fold increase compared with the highest concentration which was reached by culturing in the shaker flask, wherein the α-humulene/OD₆₀₀ ratio is constant at about 20 mg*I⁻¹/OD600.

The maximum theoretically possible yield of de novo synthesizable α-humulene per methanol is 0.26 g/g. The maximum yield of 0.031 g_{□-humulene}/g_{meOH}, achieved in fermentation 5 (see Table 3), corresponds to 12% of the maximum theoretical yield.

TABLE 3
Method properties of methanol-limited fed batch fermentations performed with
the strains AM1 and ΔcrtNb containing plasmid pFS62b. Cumate induction was effected
after attainment of the early exponential growth phase (OD₆₀₀ about 10). The values
shown represent measurements at a time point after induction.

| | | Time [h] to | max. α- | | | | |
|---|---|---|---|---|---|---|---|
| | | max. α- | humulene | | | | |
| | | humulene | concentration | | cdw | Y_{P/S}^{a} | STY^{b} |
| Strain | Fermentation | concentration | [g/l] | OD600 | [g/l] | [g/g_{MeOH}] | [mg/l * h] |
| AM1 | 1 | 63 | 0.74 | 90 | 30 | 0.023 | 11.7 |
| | 2 | 70.5 | 1.02 | 148 | n.d. | 0.024 | 15 |
| | 3 | 93 | 0.73 | 84 | 27.9 | 0.015 | 7.8 |
| CM502 | 4 | 80 | 1.37 | 79 | 28.4 | 0.023 | 17.1 |
| | 5 | 104 | 1.65 | 85 | 30 | 0.031 | 14.6 |

^{a}maximum theoretical yield is 0.26 g/g_{MeOH}
^{b}average STY after induction (t = 0) up to method end
n.d.: not determined,
cdw: cell dry weight,
STY: space time yield:

### Example 2

### Recombinant cis-Abienol Production

1 Material and Methods

1.1 Chemicals, Media and Bacterial Strains

Methylobacterium extorquens AM1 (Peel and Quayle 1961, Biochem J, 81, 465-9) was cultured at 30° C. in minimal medium according to Kiefer et al. (Kiefer et al. 2009) with 123 mM methanol.

Escherichia coli strain DH5a (Gibco-BRL, Rockville, USA) was cultured in lysogeny broth (LB) medium (Bertani 1951, J Bacteriol, 62, 293) at 37° C. Tetracycline hydrochloride was used in a concentration of 10 μg/mI for E. coil and M. extorquens. Cumate (4-isopropylbenzoic acid) was used as the inducer and used in an end concentration of 100 μM starting from a 100 mM stock solution dissolved in ethanol.

Cumate, tetracycline hydrochloride and zerumbone were purchased from Sigma-Aldrich (Steinheim, Del.). Cis-abienol was purchased from Toronto Research Chemicals (Toronto, Calif.). Dodecane was purchased from VWR (Darmstadt, Del.).

1.2 Genetic Manipulations and Plasmid Construction

The standard cloning techniques were performed according to the procedure known to those skilled in the art. The transformation of M. extorquens AM1 with plasmids was performed as described in Toyama et al. (Toyama, Anthony and Lidstrom 1998). Ribosome binding sites (RBS) were designed with the aid of the ribosome binding sites calculator (Salis 2011).

1.3 Cloning of Plasmids for the Production of Cis-Abienol

Plasmids for the synthesis of cis-abienol were constructed starting from plasmid pfs62b. For the construction of ppjo16 (pQ2148F-AbCAS-ERG20F96C-MVA), the cis-abienol synthase gene AbCAS, originally deriving from Abies balsamea (Zerbe et al. 2012, J Biol Chem, 287, 12121-31) (Accession number JN254808.1), was codon-optimized for M. extorquens AM1 with obtention of the DNA sequence according to SEQ ID No. 50. The codon-optimized gene according to SEQ ID No. 50 was amplified for insertion into pfs62b using the primers pj05 and pj25. The RBS of the AbCAS gene has the nucleic acid sequence TATTAATATTAAGAGGAGGTAATAA (SEQ ID No. 51) with a translation initiation rate (TIR) of 233,000. The gene for the GGPP synthase ERG20F96C (SEQ ID No. 52) (Ignea et al. 2015, Metabolic Engineering, 27, 65-75) from Saccharomyces cerevisiae was obtained from ERG20 by mutagenesis PCR with the primers pj26, pj16, pj17 and pj10. The TIR of the RBS of ERG20F96C was set at 10,000 and has the nucleic acid sequence CTTAAACTAACCGAGATAGGAACGAATTTTACAA (SEQ ID No. 53). Plasmid ppjo16 was constructed by insertion of the PCR products from AbCAS and ERG20F96C by Gibson cloning into the vector pfs62b. 2 cleaved with Spel and Xbal.

For the construction of plasmid ppjo17 (pQ2148F-NtLPPS-NtABS-ERG20F96C-MVA), the LPP synthase gene NtLPPS from Nicotiana tabacum (Sallaud et al. 2012, Plant J, 72, 1-17) (Accession number HE588139.1) and the cis-abienol synthase gene NtABS from Nicotiana tabacum (Sallaud et al. 2012, Plant J, 72, 1-17) (Accession number HE588140.1) were codon-optimized for M. extorquens AM1 with obtention of the DNA sequence SEQ ID No. 54 and SEQ ID No. 55 respectively. The corresponding RBS have a TIR of 145,000 for the gene NtLPPS with the DNA sequence CAACGGCCCTTACAAAAGGAGGTTAATTATT (SEQ ID No. 56) and a TIR of 130,000 for the gene NtABS with the DNA sequence GATAGAAACCCTTAATTAAGAAGGAGGTCCTTA (SEQ ID No. 57). The codon-optimized NtLPPS gene according to SEQ ID No. 54 was amplified with the primers pj05 and pj27, and for the amplification of the codon-optimized NtABS (SEQ ID No. 55) the primers pj28 and pj29 were used. For plasmid ppjo17, the gene ERG20F96C (SEQ ID No. 52) was obtained by mutagenesis PCR with the primers pj30, pj16, pj17 and pj10. The TIR of the RBS of ERG20F96C in ppjo17 was set at 9,500 and has the nucleic acid sequence AACCACTAAGAACACAGACTTATACACAGGAGGAT (SEQ ID No. 58). Plasmid ppjo17 was constructed by insertion of the PCR products from NtLPPS, NtABS and ERG20F96C by Gibson cloning into the vector pfs62b cleaved with Spel and Xbal.

An overview of the primers, plasmids and strains used is shown in Table 4.

TABLE 4
Primers, plasmids and strains used

| | | |
|---|---|---|
| Name | Description | Reference |

Primers

| | |
|---|---|
| pj05 (SEQ ID No. 70) | AACAGACAATCTGGTCTGTTTGTAAC |
| pj10 (SEQ ID No. 71) | TCTTCATCCTGCGCTCCTGTCTAGAAA |
| | TACTCTAATTAATCTATTTGCTTCTCTT |
| | GTAAACTTTG |
| pj16 (SEQ ID No. 72) | ATCGGCGACCAAGCAGTAAG |
| pj17 (SEQ ID No. 73) | TTACTGCTTGGTCGCCGATG |
| pj25 (SEQ ID No. 74) | CGTTCCTATCTCGGTTAGTTTAAGATC |
| | GATTCAGGTGGC |
| pj26 (SEQ ID No, 75) | CTTAAACTAACCGAGATAGGAACGAAT |
| | TTTACAATATGGCTTCAGAAAAAGAAAT |
| | TAGGAG |
| pj27 (SEQ ID No. 76) | GACCTCCTTCTTAATTAAGGGTTTCTAT |
| | CTACGTATCAGACCTGCTGGAAC |
| pj28 (SEQ ID No. 77) | GATAGAAACCCTTAATTAAGAAGGAGG |
| pj29 (SEQ ID No. 78) | TATAAGTCTGTGTTCTTAGTGGTTATC |
| | GATTCACGGCGAG |
| pj30 (SEQ ID No. 79) | AACCACTAAGAACACAGACTTATACAC |
| | AGGAGGATATGGCTTCAGAAAAAGAAA |
| | TTAGGAG |

Plasmids

| | |
|---|---|
| pQ2148F | Expression vector for Methylobacterium |
| | extorquens with cumate inducible |
| | promoter 2148 and adapted multiple |
| | cloning site (MCS); TetR, oriT, pBR322ori |
| pfs62b | Expression vector for Methylobacterium |
| | extorquens for synthesis of a-humulene |
| ppjo16 | Expression vector for Methylobacterium |
| | extorquens for synthesis of cis-abienol |
| | with GGPP synthase ERG20F96C and |
| | cis-abienol synthase AbCAS |
| ppjol 7 | Expression vector for Methylobacterium |
| | extorquens for synthesis of cis-abienol with |
| | GGPP synthase ERG20F96C, LPP |
| | synthase NtLPPS and cis-abienol synthase |
| | NtABS |

Strains

| | | |
|---|---|---|
| E. coli DH5α | F-, ϕ80dlacZΔM15, | ATCC |
| | Δ(lacZYA-argF)U169, deoR, | |
| | recA1, endA1, hsdR17(rK−mK+), phoA, supE44, λ−, thi- | |
| | 1 | |
| M. extorquens | Facultatively methylotrophic, | (Peel and Quayle 1961) |
| AM1 | obligatorily aerobic, gram- | DSMZ133 |
| | negative, Pink pigmented α- | |
| | proteobacterium, CmR | |

1.4 Cis-Abienol Production in Aqueous Organic Two Phase Shaker Flask Culture

Methylobacterium extorquens AM1 containing the cis-abienol production plasmids were cultured in methanol minimal medium containing tetracycline-5 hydrochloride (see above). Precultures were inoculated from agar plates into test tubes with 5 ml medium and shaken for 48 h at 30° C. and 180 rpm. Main cultures with 12 ml medium in 100 ml baffled shaker flasks were inoculated with a preculture to an OD600 of 0.1. After culturing for 16 h at 30° C. the main cultures reached the early exponential growth phase ( OD600 0.3-0.6). Next, cumate was added for the induction and 3 ml dodecane added as organic phase. After 48 h incubation, a total culture volume of 15 ml was decanted and centrifuged for 10 min at 3220 g. 1 ml of the upper dodecane layer was used for the cis-abienol analysis.

1.5 Cis-Abienol Analysis

1 ml dodecane sample was dried with NaSO4. As the internal standard, 25 μl of a dodecane solution with 1 mM zerumbone was added to 225 μl dodecane sample. Cis-abienol was analyzed and quantified by means of a GC-MS (GC17A with Q5050 mass spectrometer, Shimadzu, Kyoto, Japan), equipped with an Equity 5 column (Supelco, 30 m×0.25 mm×0.25 μM). Measurements were performed as follows: carrier gas: helium; split injection (2:1) at 250° C.; flow rate: 2.2 ml/min; interface temperature: 250° 5 C.; program: 80° C. hold for 3 mins, 16° C./min to 240° C., hold for 2 min. The retention time was 14.1 mins for cis-abienol and 11.3 mins for zerumbone. Cis-abienol in the samples was identified by comparison of three main fragmentations in the mass spectra with a commercially obtained cis-abienol standard (rel. intensity in brackets): 119 (15.9), 134 (30.3), 191 (6.0). For a quantification, a calibration curve with the concentrations 100, 50, 20, 10, 5, 2, 1 mg/L cis-abienol each with 100 μM zerumbone was used.

2 Results

2.1 Cis-Abienol Production Using Plasmids with Constitutive Promoter (Comparative Example)

For the production of cis-abienol with Methylobacterium extorquens AM1, as well as the mevalonate operon from Myxococcus xanthus the GGPP synthase ERG20F96C (a variant of the FPP synthase ERG20 from Saccharomyces cerevisiae) and further genes were expressed. GGPP should be converted either directly to cis-abienol by the bifunctional cis-abienol synthase AbCAS from Nicotiana tabacum or stepwise via the formation of LPP from GGPP by the LPP synthase NtLPPS from Nicotiana tabacum, wherein LPP should then be converted to cis-abienol by the cis-abienol synthase NtABS, likewise deriving from Nicotiana tabacum. Overall, two plasmid variants (ppjo16 and ppjo17) for the cis-abienol synthesis were constructed.

After transformation of Methylobacterium extorquens AM1 with the plasmids ppjo16 or ppjo17, the first colonies appeared after 6 days' incubation at 30° C. For AM1 with ppjo16 and ppjo17 respectively, a clone was in each case visible at this time point; in comparison to this, far more than 3,000 transformants were discernible with the empty vector pQ2148F. Only after a total of 8 days' incubation at 30° C. did further, but markedly smaller, colonies also appear with transformants with the cis-abienol production plasmids. These observations indicate that the cis-abienol production plasmids, presumably because of accumulation of prenyl phosphate intermediates toxic for Methylobacterium extorquens, markedly impair the growth of the organism, and as a result the formation of suppressors occurs.

The two transformants of AM1 with ppjo16 and ppjo17 respectively, visible after 6 days, and in each case six of the small colonies, were plated out on a fresh agar plate and incubated for 6 days at 30° C. Even with the previously small transformants, large colonies were formed after replating, i.e. suppressors. Since therefore the selective culturing of transformants with plasmid ppjo16 or ppjo17 without suppressor formation was not possible, only suppressors could be tested for product formation. For this, in order to obtain sufficient cell mass, the newly appeared suppressors were plated out onto a further, fresh agar plate and incubated for 7 days at 30° C.

The culturing was performed under the conditions described above as aqueous organic two phase cultures, wherein dodecane was used as the organic phase.

A suppressor mutant of M. extorquens AM1 containing plasmid ppjo16 (named 16s6) was capable of producing cis-abienol as is shown by the peak with the same retention time and mass spectrum in comparison to the cis-abienol standard in FIG. 1. In contrast to this, no cis-abienol was detectable for M. extorquens AM1 with the empty vector control (pQ2148F). For the suppressor mutant 16s6 of M. extorquens AM1 with ppjo16, 21.1 mg/L cis-abienol were measured in the dodecane phase, which corresponds to a product concentration of 5.3 mg/L cis-abienol in the culture broth. After plasmid isolation of ppjo16 from the suppressor mutant 16s6 and subsequent sequencing of the plasmid, the mutation giving rise to the suppressor could be identified. In the promoter region of the plasmid, exactly 115 nucleotides before the start codon of the AbCAS gene, a sequence of a total of 28 nucleotides was deleted. SEQ ID No. 59 represents the sequence of the promoter region in the plasmid ppjo16, while the mutated promoter sequence in plasmid ppjo16 from the suppressor mutant 16s6 is recorded under SEQ ID No. 60.

### Example 3

### Recombinant Production of Santalene

1 Material and Methods

1.1 Chemicals, Media and Bacterial Strains

Methylobacterium extorquens AM1 (Peel and Quayle 1961, Biochem J, 81, 465-9) was cultured at 30° C. in minimal medium according to Kiefer et al. (Kiefer et al., PLoS One, e7831) with 123 mM methanol.

Escherichia coli strain DH5a (Gibco-BRL, Rockville, USA) was cultured in lysogeny broth (LB) medium (Bertani 1951) at 37° C. Tetracycline hydrochloride was used in a concentration of 10 pg/ml for E. coli and M. extorquens. Cumate (4-isopropylbenzoic acid) was used as the inducer and dissolved in ethanol was used in an end concentration of 100 μM, starting from a 100 mM stock solution.

Cumate, tetracycline hydrochloride, zerumbone and sandalwood oil were purchased from Sigma-Aldrich (Steinheim, Del.). Dodecane was purchased from VVVR (Darmstadt, Del.).

1.2 Genetic Manipulations and Plasmid Construction

The standard cloning techniques were performed according to the procedure known to those skilled in the art. The transformation of M. extorquens AM1 with plasmids was performed as described in Toyama et al. (Toyama et al., FEMS Microbiology Letters, 166, 1-7). Ribosome binding sites (RBS) were designed by means of the ribosome binding site calculator (Sails 2011, Methods in Enzymology, ed. V. Christopher, 19-42. Academic Press).

1.3 Cloning of Plasmids for Production of Santalene

Plasmids for the synthesis of santalene were constructed starting from the plasmids pQ2418F, pfs60b and pfs62b.

For the construction of ppjo01woMVA (pQ2148F-SSpiSSY-ERG20) and ppjo01 (pQ2148F-SSpiSSY-ERG20-MVA), the santalene synthase gene SSpiSSY, originally deriving from Santalum spicatum (Jones et al., 2011, Journal of Biological Chemistry, 286, 17445-17454) (Accession number HQ343278.1), was codon-optimized for M. extorquens AM1 with obtention of the DNA sequence according to SEQ ID No. 61. The codon-optimized gene according to SEQ ID No. 61 was amplified for insertion into pfs60b (Sonntag et al. 2015) using the primers SSpiSSY_RBSopt_fw and SSpiSSY_rev. The SSpiSSY PCR product was digested with Spel and Clal and inserted into identically digested plasmid pfs60b, yielding plasmid ppjo01_woMVA. Plasmid ppjo01 was constructed by cleaving the genes SSpiSSY and ERG20 out from ppjo01_woMVA with Xbal and EcoRl, followed by the insertion into identically digested pfs62b. In both plasmids, ppjo01 and ppjo01_woMVA, SSpiSSY had the nucleic acid sequence TGTTACACCCACAGAACAAACCCGAGGTAACT (SEQ ID No. 62) with a TIR of 44,000, the TIR of the RBS of ERG20 possessed the nucleic acid sequence ACATCAAACCAAAGGACTTTACAGGTAGTAGAA (SEQ ID No. 63) with a TIR of 20,000.

For the construction of ppjo03 (pQ2148F-SanSyn-ERG20), the santalene synthase gene SanSyn, originally deriving from Clausena lansium (Scalcinati et al., 2012, Metabolic Engineering, 14, 91-103; Scalcinati et al., 2012, Microb Cell Fact, 11, 117) (Accession number HQ452480.1), was codon-optimized for M. extorquens AM1 with obtention of the DNA sequence according to SEQ ID No. 64. The codon-optimized gene according to SEQ ID No. 64 was amplified for insertion into pfs62b using the primers pj05 and pj06. The RBS of the SanSyn gene had the nucleic acid sequence GAAGAAGGAGGTAGTCATAAAGAAGGAGGTAACTA (SEQ ID No. 65) with a TIR of 233,000. Plasmid ppjo03 was constructed by insertion of the PCR product from SanSyn by Gibson cloning into the vector pfs62b cleaved with Spel and Bsu36I. The TIR of the RBS of ERG20 is set at 22,000 and had the nucleic acid sequence TCCCCAGCGCGCCCCCCAATTCAGGATAACATAG (SEQ ID No. 66).

For the construction of ppjo04_woMVA (pQ2148F-ERG20fusSSpiSSY) and ppjo04 (pQ2148F-ERG20fusSSpiSSY-MVA), the FPP synthase gene ERG20 with C-terminal (GGGGS)x2 linker was amplified with the primers ERG20-fus_fw and ERG20-fus_rev for insertion into pQ2418F (Sonntag et al., Metab Eng, 32, 82-94). The gene SSpiSSY (SEQ ID No. 61) was amplified with the primers SSpiSSY_RBSopt_fw and SSpiSSY_rev, digested with BamHI and EcoRl and inserted into identically digested plasmid pQ2418F-ERG20fus, yielding plasmid ppjo04_woMVA. Plasmid ppjo04 was constructed by cleaving the gene ERG20fus out from ppjo04_woMVA with Asel and EcoRI, followed by insertion into identically digested pfs62b. In both plasmids, ppjo04 and ppjo04woMVA, ERG20 had the nucleic acid sequence AAACATAGCATATTAGCAGATTAAGGACATACGT (SEQ ID No. 67) with a TIR of 53,000.

For the construction of ppjo05 (pQ2148F-SSpiSSYfusERG20-MVA), the codon-optimized santalene synthase gene SspiSSY (SEQ ID No. 61) was amplified with the primers pj01 and pj08. The gene for the FPP synthase ERG20 with N-terminal (GGGGS)×2 linker was amplified using the primers pj09 and pj10. The TIR of the fusion protein was set at 402,000 and had the nucleic acid sequence CCCCTTCCCTTATTTAAACCAGAGGAGGTAACAAA (SEQ ID No. 68). Plasmid ppjo05 was constructed by insertion of the PCR products from SSpiSSY and fusERG20 by Gibson cloning into the vector pfs62b cleaved with Spel and Xbal.

For the construction of ppjo06 (pQ2148F-SSpiSSY-ERG20_RBSmax), the codon-optimized santalene synthase gene SSpiSSY (SEQ ID No. 61) was amplified with the primers pj01 and pj77. The optimized RBS of the SSpiSSY gene had the nucleic acid sequence according to SEQ ID No. 68 with a TIR of 402,000. The gene for the FPP synthase ERG20 was amplified using the primers pj10 and pj78. The TIR of ERG20 was set at 1,344,000 and had the nucleic acid sequence AACCAAATAGGATTAGCACAGAAGGGGGTAATA (SEQ ID No. 69). Plasmid ppjo06 was constructed by insertion of the PCR products from SSpiSSY and ERG20 by Gibson cloning into the vector pfs62b cleaved with Spel and Xbal

The TIR of the RBS of the hmgs gene was maintained at 189 in the plasmids ppjo01, ppjo03 and ppjo04 similarly to the humulene synthesis plasmid pfs62b. For the plasmids ppjo05 and ppjo06, the TIR value of the RBS of the hmgs was set at 6345.

An overview of the primers, plasmids and strains used is given in Table 5.

TABLE 5
Primers, plasmids and strains used

| | | |
|---|---|---|
| Name | Description | Reference |

Primers

| | |
|---|---|
| SspiSSY_RBSopt_fw | ACGAACTAGTTGTTACACCCACAGAACAAACCCGA |
| (SEQ ID No. 80) | GGTAACTATGGACTCGTCGACCGCC |
| SspiSSY_rev (SEQ | ATCGTATCGATTCACTCCTCGCCGAGCGG |
| ID No. 81) | |
| pj01 (SEQ ID No. | GACAATCTGGTCTGTTTGTAACTAGTCCCCTTCCCT |
| 82) | TATTTAAACCAGAGGAGGTAACAAAATGGACTCGTC |
| | GACCGCCAC |
| pj05 (SEQ ID No. | AACAGACAATCTGGTCTGTTTGTAAC |
| 70) | |
| pj06 (SEQ ID No. | TGGGCATACCAGTCACATGC |
| 83) | |
| ERG20-fus_fw | ACGAACTAGTAAACATAGCATATTAGCAGATTAAGG |
| (SEQ ID No. 84) | ACATACGTATGGCTTCAGAAAAAGAAATTAG |
| ERG20-fus_rev | ACTAGGATCCGCCGCCACCCGAGCCACCGCCACC |
| (SEQ ID No. 85) | TTTGCTTCTCTTGTAAACTTTG |
| pj08 (SEQ ID No. | TTTCTGAAGCCATGGATCCGCCGCCACCCGAGCCA |
| 86) | CCGCCACCCTCCTCGCCGAGCGGGATC |
| pj09 (SEQ ID No. | GGATCCATGGCTTCAGAAAAAGAAATTAGGAG |
| 87) | |
| pj10 (SEQ ID No. | TCTTCATCCTGCGCTCCTGTCTAGAAATACTCTAAT |
| 71) | TAATCTATTTGCTTCTCTTGTAAACITTG |
| pj77 (SEQ ID No. | CTTCTGTGCTAATCCTATTTGGTTATCGATTCACTC |
| 88) | CTCGCCGAGC |
| pj78 (SEQ ID No. | GATAACCAAATAGGATTAGCACAGAAGGGGGTAAT |
| 89) | AATGGCTTCAGAAAAAGAAATTAGGAG |

Plasmids

| | | |
|---|---|---|
| pQ2148F | Expression vector for Methylobacterium extorquens | (Sonntag et |
| | with cumate inducible promoter 2148 and adapted | al., 2015, |
| | multiple cloning site (MCS); TetR, oriT, pBR322ori | Metab Eng, |
| | | 32, 82-94) |
| pfs60b | Expression vector for Methylobacterium extorquens for | (Sonntag et |
| | synthesis of α-humulene, without genes coding for | al., 2015, |
| | proteins of the mevalonate pathway | Metab Eng, |
| | 32, 82-94) | |
| pfs62b | Expression vector for Methylobacterium extorquens for | (Sonntag et |
| | synthesis of α-humulene | al., 2015, |
| | | Metab Eng, |
| | | 32, 82-94) |
| ppjo01_woMVA | Expression vector for Methylobacterium extorquens for | |
| | synthesis of santalene with EPP synthase ERG20 and | |
| | santalene synthase SspiSSY, without genes coding for | |
| | proteins of the mevalonate pathway | |
| ppjo01 | Expression vector for Methylobacterium extorquens for | |
| | synthesis of santalene with EPP synthase ERG20 and | |
| | santalene synthase SspiSSY | |
| ppj003 | Expression vector for Methylobacterium extorquens for | |
| | synthesis of santalene with EPP synthase ERG20 and | |
| | santalene synthase SanSyn | |
| ppjo04_woMVA | Expression vector for Methylobacterium extorquens for | |
| | synthesis of santalene with a fusion protein from the | |
| | FFP synthase ERG20 and the santalene synthase | |
| | SSpiSSY (ERG20fusSSpiSSY), without genes coding | |
| | for proteins of the mevalonate pathway | |
| ppjo04 | Expression vector for Methylobacterium extorquens for | |
| | synthesis of santalene with a fusion protein from the | |
| | EPP synthase ERG20 and the santalene synthase | |
| | SSpiSSY (ERG20fusSSpiSSY) | |
| ppjo05 | Expression vector for Methylobacterium extorquens for | |
| | synthesis of santalene with a fusion protein from the | |
| | santalene synthase SSpiSSY and the FPP synthase | |
| | ERG20 (SSpiSSYfusERG20) | |
| ppj006 | Expression vector for Methylobacterium extorquens for | |
| | synthesis of santalene with FPP synthase ERG20 and | |
| | santalene synthase SspiSSY, wherein the TIR of the | |
| | RBS of ERG20 was set maximally high | |

Strains

| | | |
|---|---|---|
| E. coli DH5a | F-, ϕ80dlacZΔM15, Δ(lacZYA-argF)U169, deoR, recA1, | ATCC |
| | endA1, hsdR17(rk−mk+), phoA, supE44, λ−, thi-1 | |
| M. extorquens | Facultatively methylotrophic, obligatorily aerobic, gram- | (Peel and |
| AM1 | negative, Pink pigmented α-proteobacterium, CmR | Quayle |
| | | 1961, |
| | | Biochem J, |
| | | 81, 465-9.) |
| | | DSMZ1338 |

1.4 Santalene Production in Aqueous Organic Two Phase Shaker Flask Culture

Methylobacterium extorquens AM1 containing the santalene production plasmids was cultured in methanol minimal medium containing tetracycline-5 hydrochloride (see above). Precultures were inoculated from agar plates into test tubes with 5 ml medium and shaken for 48 h at 30° C. and 180 rpm. Main cultures with 12 ml medium in 100 ml baffled shaker flasks were inoculated with a preculture to an OD₆₀₀ of 0.1. After culturing for 16 h at 30° C. the main cultures reached the early exponential growth phase (OD₆₀₀ 0.3-0.6). Next, cumate was added for the induction and 3 ml dodecane added as organic phase. After 48 h incubation, a total culture volume of 15 ml was decanted and centrifuged for 10 mins at 3220 g. 1 ml of the upper dodecane layer was used for the santalene analysis.

1.5 Santalene Analysis

1 ml dodecane sample was dried with NaSO₄. As the internal standard, 25 μl of a dodecane solution with 1 mM zerumbone were added to 225 μl of dodecane sample.

Santalene was analyzed by means of a GC-MS (GC17A with Q5050 mass spectrometer, Shimadzu, Kyoto, Japan), equipped with an Equity 5 column (Supelco, 30 m×0.25 mm×0.25 μM). Measurements were performed as follows: carrier gas: helium; split injection (8:1) at 250° C.; flow rate: 2.2 ml/min; interface temperature: 250° C.; program: 80° C. hold for 3 mins, 16° C./min to 240° C., hold for 2 mins. Since a santalene standard is not commercially available, sandalwood oil was used instead of this for the analysis of santalene products in the samples. Before the measurement, the sandalwood oil was diluted 1:500 in dodecane. The various substances contained in sandalwood oil eluted between 11 and 12.4 mins.

2 Results

2.1 Santalene Production Using Plasmids with Constitutive Promoter (Comparative Example)

For the production of santalene with Methylobacterium extorquens AM1, as well as the mevalonate operon from Myxococcus xanthus, the FPP synthase ERG20 from Saccharomyces cerevisiae and further genes were expressed. FPP should be converted to santalene by a santalene synthase from Santalum spicatum (SSpiSSY) or Clausena Iansium (SanSyn). Also, fusion proteins from the santalene synthase SSpiSSY and the FPP synthase ERG20 were tested for santalene production. In total, seven plasmid variants (ppjo01, ppjo01_woMVA, ppjo03, ppjo04, ppjo04_woMVA, ppjo05, ppjo06) were constructed for santalene synthesis.

After transformation of Methylobacterium extorquens AM1 with the santalene production plasmids, colonies appeared after 5 days' incubation at 30° C. For plasmids without mevalonate pathway (pQ2418F, ppjo01_woMVA, ppjo04_woMVA) and those in which the TIR of the RBS hmgs was set at 189, far more than 3,000 transformants were visible. In comparison to this, with plasmids with a TIR of the RBS of the hmgs of 6345 (ppjo05, ppjo06) with circa 100 visible colonies, markedly fewer transformants appeared. After a total of 8 days' incubation at 30° C., further, but markedly smaller, colonies appeared with transformants with ppjo05 and ppjo06 respectively. These observations indicated that the santalene production plasmids with a higher set TIR of the RBS of the hmgs markedly impaired the growth of the organism, presumably because of accumulation of prenyl phosphate intermediates toxic to Methylobacterium extorquens , and as a result formation of suppressors occurred.

The transformants from AM1 with ppjo05 and ppjo06 respectively, visible after 5 days, and in each case six of the smaller colonies, were plated out onto a fresh agar plate and incubated for 6 days at 30° C. Even with the previously small transformants, after replating large colonies, i.e. suppressors, formed. Since therefore the selective culturing of transformants with plasmid ppjo05 or ppjo06 without suppressor formation was not possible, only suppressors could be tested for product formation. For this, for the obtention of sufficient cell mass, the newly appeared suppressors were plated out onto a further, fresh agar plate and incubated for 7 days at 30° C. For the other strains of M. extorquens, in which suppressor formation did not occur, likewise in each case 3 different clones were plated out onto a new agar plate and incubated for 7 days at 30° C.

The culturing was performed under the conditions described above as aqueous organic two phase cultures, wherein dodecane was used as the organic phase.

M. extorquens AM1 containing the plasmids ppjo01_woMVA, ppjo03, ppjo04, ppjo04_woMVA or ppjo05 was capable of producing santalene as was shown by the α-santalene peak with identical retention time and mass spectrum in comparison to substances in the sandalwood oil in FIG. 1. By way of example, the chromatogram and mass spectrum of a sample from M. extorquens AM1 containing the plasmid ppjo03 is shown. In contrast to this, for M. extorquens AM1 with the empty vector control (pQ2148F) no santalene was detectable.

Those skilled in the art recognize that the bacterial strains and fermentation conditions described here in the practical examples can readily be adapted without departing from the scope of the invention. Thus simple adaptations are conceivable for the production of other sesquiterpenes from methanol or ethanol, for example potential biofuels, such as bisabolene, or of fragrance substances such as santalene or of diterpenes such as sclareol. The invention enables the bioproduction of terpenes from the carbon source methanol or ethanol not competing with foods.

All characteristics and advantages, including constructive details, spatial arrangements and method steps following from the claims, the descriptions and the drawing can be material to the invention in themselves and also in a great variety of combinations.

### CITED NON-PATENT LITERATURE

Ajikumar, P. K., Tyo, K., Carlsen, S., Mucha, O., Phon, T. H. Stephanopoulos, G., 2008. terpenoids: opportunities for biosynthesis of natural product drugs using engineered microorganisms. Molecular pharmaceutics. 5, 167-90

Asadollahi, M. A., Maury, J., Moller, K., Nielsen, K. F., Schalk, M., Clark, A., Nielsen, J., 2008. Production of plant sesquiterpenes in Saccharomyces cerevisiae: Effect of ERG9 repression on sesquiterpene biosynthesis. Biotechnology and Bioengineering. 99, 666-677;

Bertani, G. (1951) STUDIES ON LYSOGENESIS I.: The Mode of Phage Liberation by Lysogenic Escherichia coli1. J Bacteriol, 62, 293.

Caniard, Anne, et al. “Discovery and functional characterization of two diterpene synthases for sclareol biosynthesis in Salvia sclarea (L.) and their relevance for perfume manufacture.” BMC plant biology 12.1 (2012): 119.

Chandran, S., Kealey, J., Reeves, C., 2011. Microbial production of isoprenoids. Process Biochemistry. 46, 1703-1710

Chou, H. H., Marx, C. J., 2012. Optimization of gene expression through divergent mutational paths. Cell reports. 1, 133-40.

Entian, K.-D., Koetter, P., 1998. Yeast mutant and plasmid collections. In: Brown, A. J. P., Tuite, M. F., Eds.). Academic Press Ltd., San Diego, pp. 431-449.

Ignea, C., F. A. Trikka, K. Nikolaidis, P. Georgantea, E. Ioannou, S. Loupassaki, P. Kefals, A. K. Kanellis, V. Roussis, A. M. Makris & S. C. Kampranis (2015) Efficient 10 diterpene production in yeast by engineering Erg20p into a geranylgeranyl diphosphate synthase. Metabolic Engineering, 27, 65-75.

Jones, C. G., J. Moniodis, K. G. Zulak, A. Scaffidi, J. A. Plummer, E. L. Ghisalberti, E. L. Barbour & J. Bohlmann (2011) Sandalwood Fragrance Biosynthesis Involves Sesquiterpene Synthases of Both the Terpene Synthase (TPS)-a and TPS-b Subfamilies, including Santalene Synthases. Journal of Biological Chemistry, 286, 17445-17454.

Kaczmarczyk, A., Vorholt, J. A., Francez-Charlot, A., 2013. Cumate-inducible gene expression system for sphingomonads and other Alphaproteobacteria. Appl. Environ. Microbiol. 79, 6795-802.

Kiefer, P., Buchhaupt, M., Christen, P., Kaup, B., Schrader, J., Vorholt, J. A., 2009. Metabolite Profiling Uncovers Plasmid-Induced Cobalt Limitation under Methylotrophic Growth Conditions. PLoS ONE. 4, e7831.

Martin, V. J., Pitera, D. J., Withers, S. T., Newman, J. D., Keasling, J. D., 2003. Engineering a mevalonate pathway in Escherichia coli for production of terpenoids. Nature biotechnology. 21, 796-802;

Marx, C. J., Lidstrom, M. E., 2001. Development of improved versatile broad-host-range vectors for use in methylotrophs and other Gram-negative bacteria. Microbiology. 147, 2065-2075.

Mi, J., Becher, D., Lubuta, P., Dany, S., Tusch, K., Schewe, H., Buchhaupt, M., Schrader, J., 2014. De novo production of the monoterpenoid geranic acid by metabolically engineered Pseudomonas putida. Microbial cell factories. 13, 170.

Peel, D. & J. R. Quayle (1961) Microbial growth on C1 compounds. I. Isolation and characterization of Pseudomonas AM 1. Biochem J, 81, 465-9.

Peralta-Yahya, P. P., Keasling, J. D., 2010. Advanced biofuel production in microbes. Biotechnol J. 5, 147-62;

Puigbo, P., Bravo, I., Garcia-Valive, S., 2008. E-CAI: a novel server to estimate an expected value of Codon Adaptation Index (eCAI). BMC Bioinformatics. 9, 65.

Salis, H. M. 2011. Chapter two—The Ribosome Binding Site Calculator. In Methods in Enzymology, ed. V. Christopher, 19-42. Academic Press.

Sallaud, C., C. Giacalone, R. Topfer, S. Goepfert, N. Bakaher, S. Rosti & A. Tissier (2012) Characterization of two genes for the biosynthesis of the labdane diterpene Z-abienol in tobacco (Nicotiana tabacum) glandular trichomes. Plant J, 72, 1-17.

Sarria, S., Wong, B., Martin, H., Keasling, J. D., Peralta-Yahya, P., 2014. Microbial synthesis of Pinene. ACS Synthetic Biology 3 (7), 466-475

Scalcinati, G., C. Knuf, S. Partow, Y. Chen, J. Maury, M. Schalk, L. Daviet, J. Nielsen & V. Siewers (2012) Dynamic control of gene expression in Saccharomyces cerevisiae engineered for the production of plant sesquiterpene α-santalene in a fed batch mode. Metabolic Engineering, 14, 91-103.

Scalcinati, G., S. Partow, V. Siewers, M. Schalk, L. Daviet & J. Nielsen (2012) Combined metabolic engineering of precursor and co-factor supply to increase alpha-santalene production by Saccharomyces cerevisiae. Microb Cell Fact, 11, 117.

Sonntag, F., C. Kroner, P. Lubuta, R. Peyraud, A. Horst, M. Buchhaupt & J. Schrader (2015) Engineering Methylobacterium extorquens for de novo synthesis of the sesquiterpenoid alpha-humulene from methanol. Metab Eng, 32, 82-94.

Toyama, H., Anthony, C., Lidstrom, M. E., 1998. Construction of insertion and deletion mxa mutants of Methylobacterium extorquens AM1 by electroporation. FEMS Microbiol. Lett. 166, 1-7.

Van Dien, S. J., Marx, C. J., O'Brien, B. N., Lidstrom, M. E., 2003. Genetic characterization of the carotenoid biosynthetic pathway in Methylobacterium extorquens AM1 and isolation of a colorless mutant. Appl. Environ. Microbiol. 69, 7563-6.

Yoon, S.-H., Lee, S.-H., Das, A., Ryu, H.-K., Jang, H.-J., Kim, J.-Y., Oh, D.-K., Keasling, J. D., Kim, S.-W., 2009. Combinatorial expression of bacterial whole mevalonate pathway for the production of β-carotene in E. coli. Journal of Biotechnology. 140, 218-226;

Yu, F., Okamto, S., Nakasone, K., Adachi, K., Matsuda, S., Harada, H., Misawa, N., Utsumi, R., 2008. Molecular cloning and functional characterization of alpha-humulene synthase, a possible key enzyme of zerum bone biosynthesis in shampoo ginger (Zingiber zerumbet Smith). Planta. 227, 1291-9.

Zerbe, P., A. Chiang, M. Yuen, B. Hamberger, B. Hamberger, J. A. Draper, R. Britton & J. Bohlmann (2012) Bifunctional cis-abienol synthase from Abies balsamea discovered by transcriptome sequencing and its implications for diterpenoid fragrance production. J Biol Chem, 287, 12121-31.

### SEQUENCE PROTOCOL—FREE TEXT

SEQ ID No. 1: Hydroxymethylglutaryl-CoA synthase, Myxococcus Xanthus
SEQ ID No. 2: Hydroxymethylglutaryl-CoA reductase, Myxococcus Xanthus
SEQ ID No, 3: Mevalonate kinase, Myxococcus Xanthus
SEQ ID No. 4; Phosphomevalonate kinase, Myxococcus Xanthus
SEQ ID No. 5: Pyrophosphomevalonate decarboxylase, Myxococcus Xanthus
SEQ ID No. 6: Isopentenyl pyrophosphate isomerase, Myxococcus Xanthus
SEQ ID No. 7: hmgs gene from Myxococcus xanthus with removed EcoRI restriction site with insertion of a silent mutation (gaattc to gagttc)
SEQ ID No. 8: hmgr gene from Myxococcus xanthus
SEQ ID No. 9; mvaK1 gene from Myxococcus xanthus
SEQ ID No, 10: mvaK2 gene from Myxococcus xanthus
SEQ ID No. 11; mvaD gene from Myxococcus xanthus
SEQ ID No. 12. fni gene from Myxococcus xanthus
SEQ ID No. 13: FPP synthase ERG20 from Saccharomyces cerevisiae, PRT
SEQ ID No. 14; FPP synthase ERG20 from Saccharomyces cerevisiae, DNA
SEQ ID No. 15: Sesquiterpene synthase from Zingiber zerumbet
SEQ ID No, 16: DNA sequence of the alphα-humulene synthase zssl from Zingiber zerumbet codon-optimized for Methylobacterium extorquens AM1
SEQ ID No. 17 Primer HMGS-fw
SEQ ID No. 18 Primer HMGS-rev
SEQ ID No. 19 Primer HMGS-over-fw
SEQ ID No. 20 Primer HMGS-over-rev
SEQ ID No. 21 Primer MVA1_fw
SEQ ID No, 22 Primer MVA-SaclA-rev
SEQ ID No. 23 Primer MVA-SaclA-fw
SEQ ID No. 24 Primer MVA-SaclB-rev
SEQ ID No. 25 Primer MVA-SaclB_fw
SEQ ID No. 26 Primer MVA2_rev
SEQ ID No. 27 Primer pQF_MCS-fw
SEQ ID No. 28 Primer pQF_MCS-rev
SEQ ID No. 29 Primer ZSSI-fw
SEQ ID No. 30 Primer ZSSI-RBS-fw
SEQ ID No. 31 Primer ZSSI-rev
SEQ ID No. 32 Primer ERG20_fw
SEQ ID No. 33 Primer ERG20-RB S(35k)-fw
SEQ ID No. 34 Primer ERG20-RB S(20k)-fw
SEQ ID No. 35 Primer ERG20 rev
SEQ ID No. 36 Primer ERG20 rev-2
SEQ ID No. 37 Primer fni-RBSopt-fw
SEQ ID No. 38 Primer fni-RBSopt-rev
SEQ ID No. 39 optimized RBS of ERG20 with a TIR of 22,000
SEQ ID No. 40 optimized RBS of ERG20 with a TIR of 36,800
SEQ ID No. 41 optimized RBS of zssl with a TIR of 221,625
SEQ ID No. 42: GGPP synthase from S. cerevisiae
SEQ ID No. 43: GGPP synthase from Pantoea agglomerans
SEQ ID No. 44: GGPP synthase from Taxus canadensis
SEQ ID No. 45 Sesquiterpene synthase from Santalum album
SEQ ID No. 46: Sesquiterpene synthase Santalum spicatum
SEQ ID No. 47: Diterpene synthase from Abies balsamea
SEQ ID No. 48 optimized RBS of fni with a TIR of 65,000
SEQ ID No. 49 optimized RBS of hmgs with a TIR of 6,345
SEQ ID No, 50 DNA sequence of the cis-abienol synthase AbCAS from Abies balsamea codon-optimized for Methylobacterium extorquens AM1
SEQ ID No. 51 optimized RBS of AbCAS with a TIR of 233,000
SEQ ID No. 52 DNA sequence of the GGPP synthase ERG20F96C from Saccharomyces cerevisiae
SEQ ID No. 53 optimized RBS of ERG20F96C with a TIR of 10,000 in plasmid ppjo16
SEQ ID No. 54 DNA sequence of the LPP synthase NtLPPS gene from Abies balsamea codon-optimized for Methylobacterium extorquens AM1
SEQ ID No. 55 DNA sequence of the cis-abienol synthase NtABS gene from Abies balsamea codon-optimized for Methylobacterium extorquens AM1
SEQ ID No. 56 optimized RBS of NtLPPS with a TIR of 145,000 in plasmid ppjo16
SEQ ID No. 57 optimized RBS of NtABS with a TIR of 130,000 in plasmid ppjo16
SEQ ID No. 58 optimized RBS of ERG20F96C with a TIR of 9,500 in plasmid ppjo17
SEQ ID No. 59: Promoter region of plasmid ppjo16 including RBS AbCAS, beginning directly after CymR*
SEQ ID No. 60:. Promoter region of plasmid ppjo16 from clone 16s6 including RBS AbCAS, beginning directly after CymR*
SEQ ID No. 61: DNA sequence of the santalene synthase SspiSSY from Santalum spicatum codon-optimized for Methylobacterium extorquens AM1
SEQ ID No. 62: optimized RBS of SSpiSSY with a TIR of 44,000 in plasmid ppjo01 and ppjo01_woMVA
SEQ ID No. 63; optimized RBS of ERG20 with a TIR of 20,000 in plasmid ppjo01 and ppjo01_woMVA
SEQ ID No. 64 DNA sequence of the santalene synthase SanSyn from Clausena lansium codon-optimized for Methylobacterium extorquens AM1
SEQ ID No. 65: optimized RBS of SanSyn with a TIR of 233,000 in plasmid ppjo03
SEQ ID No. 66: optimized RBS of ERG20 with a TIR of 22,000 in plasmid ppjo03
SEQ ID No. 67: optimized RBS of ERG20 with a TIR of 53,000 in plasmid ppjo04 and ppjo04_woMVA
SEQ ID No. 68: optimized RBS of SSpiSSY with a TIR of 402,000 in plasmids ppjo05 and ppjo06
SEQ ID No. 69: optimized RBS of ERG20 with a TIR of 1,344,000 in plasmid ppjo06
SEQ ID No. 70 Primer pj05
SEQ ID No. 71 Primer pj10
SEQ ID No. 72 Primer pj16
SEQ ID No. 73 Primer pj17
SEQ ID No. 74 Primer pj25
SEQ ID No. 75 Primer pj26
SEQ ID No. 76 Primer pj27
SEQ ID No. 77 Primer pj28
SEQ ID No. 78 Primer pj29
SEQ ID No. 79 Primer pj30
SEQ ID No. 80 Primer SspiSSY_RBSopt_fw
SEQ ID No. 81 Primer SspiSSY_rev
SEQ ID No. 82 Primer pj01
SEQ ID No. 83 Primer pj06
SEQ ID No. 84 Primer ERG20-fus_fw
SEQ ID No. 85 Primer ERG20-fus_rev
SEQ ID No. 86 Primer pj08
SEQ ID No. 87 Primer pj09
SEQ ID No. 88 Primer pj77
SEQ ID No. 89 Primer pj78
SEQ ID No. 90 optimized RBS of hmgs with a TIR of 189

1. A methylotrophic bacterium containing a heterologous terpene synthase and recombinant DNA coding for at least one polypeptide with enzymatic activity for expression in said bacterium, characterized in that said at least one polypeptide with enzymatic activity is selected from the group consisting of
at least one enzyme of a heterologous mevalonate pathway selected from the group consisting of hydroxymethylglutaryl-CoA synthase (HMG-CoA synthase), hydroxymethylglutaryl-CoA reductase (HMG-CoA reductase), mevalonate kinase, phosphomevalonate kinase, pyrophosphomevalonate decarboxylase and isopentenyl pyrophosphate isomerase; and
a synthase of a prenyl diphosphate precursor.

2. A methylotrophic bacterium containing a heterologous hydroxymethylglutaryl-CoA synthase (HMG-CoA synthase) and a hydroxymethylglutaryl-CoA reductase (HMG-CoA reductase) as enzymes of a heterologous mevalonate pathway and recombinant DNA coding for at least one polypeptide with enzymatic activity for expression in said bacterium, characterized in that said at least one polypeptide with enzymatic activity is selected from the group consisting of
at least one further enzyme of a heterologous mevalonate pathway selected from the group consisting of mevalonate kinase, phosphomevalonate kinase, pyrophosphomevalonate decarboxylase and isopentenyl pyrophosphate isomerase;
a heterologous terpene synthase and
a synthase of a prenyl diphosphate precursor.

3. The bacterium according to claim 1 or 2, characterized in that the at least one enzyme of the heterologous mevalonate pathway contains a peptide sequence with an identity of respectively at least 60% to the peptide sequence according to SEQ ID No. 1, SEQ ID No. 2, SEQ ID No. 3, SEQ ID No. 4, SEQ ID No. 5 or SEQ ID No. 6.

4. The bacterium according to any one of claims 1 to 3, characterized in that the heterologous terpene synthase is selected from the group consisting of a sesquiterpene synthase and a diterpene synthase.

5. The bacterium according to claim 4, characterized in that the heterologous terpene synthase is a sesquiterpene synthase, wherein the sesquiterpene synthase is an enzyme for the synthesis of a cyclic sesquiterpene, the sesquiterpene in particular is selected from the group consisting of α-humulene and epimers of santalene, such as α-santalene, β-santalene, epi-β-santalene or α-exo-bergamotene, and bisabolenes, such as b-bisabolene.

6. The bacterium according to claim 4, characterized in that the heterologous terpene synthase is a diterpene synthase, in particular an enzyme for the synthesis of a diterpene, the diterpene in particular selected from the group consisting of sclareol, cis-abienol, abitadiene, isopimaradiene, manool and larixol.

7. The bacterium according to any one of claims 1 to 6, characterized in that the synthase of a prenyl diphosphate precursor is an enzyme selected from the group consisting of farnesyl diphosphate synthase (FPP synthase) and gerany/geranyl diphosphate synthase (GGPP synthase).

8. The bacterium according to any one of 1 to 7, characterized in that the synthase of a prenyl diphosphate precursor is a heterologous FPP synthase, wherein the heterologous FPP synthase is a eukaryotic or prokaryotic FPP synthase.

9. The bacterium according to any one of 1 to 7, characterized in that the synthase of a prenyl diphosphate precursor is a heterologous GGPP synthase, wherein the heterologous GGPP synthase is an enzyme from an organism which is selected from the group consisting of bacteria, plants and fungi.

10. The bacterium according to any one of claims 1 to 9, characterized in that the recombinant DNA for heterologous expression of said enzymes is provided with a common inducible promoter or several mutually independently inducible promoters.

11. The bacterium according to any one of claims 1 to 10, characterized in that the recombinant DNA is in each case mutually independently expressible on plasmid or chromosomally.

12. The bacterium according to any one of claims 1 to 11, characterized in that the bacterium is a methylotrophic proteobacterium, in particular a bacterium of the genus Methylobacterium or of the genus Methylomonas , preferably the bacterium Methylobacterium extorquens.

13. The bacterium according to any one of claims 1 to 12, characterized in that the bacterium is a strain lacking carotenoid biosynthesis activity, in particular lacking diapolycopene oxidase activity.

14. A method for de novo microbial synthesis of sesquiterpenes or diterpenes from methanol and/or ethanol, comprising the following steps:
providing a methanol and/or ethanol-containing aqueous medium,
culturing a methylotrophic bacterium according to any one of claims 1 to 13 in said medium in a bioreactor, wherein methanol and/or ethanol is converted into a terpene by the bacterium,
separating the sesquiterpene or diterpene formed in the bioreactor.

15. The method according to claim 14, characterized in that in said medium methanol and/or ethanol is/are contained as the sole carbon source(s) for culturing said bacterium.

16. Use of a methanol and/or ethanol-containing medium for culturing a recombinant methylotrophic bacterium according to any one of claims 1 to 13 for the de novo microbial synthesis of sesquiterpenes or diterpenes from methanol and/or ethanol.

17. Use of a methylotrophic bacterium according to any one of claims 1 to 13 for the de novo microbial synthesis of sesquiterpenes or diterpenes from methanol and/or ethanol.
